(19) United States
(12) Patent Application Publication
(10) Pub. No.: US 2014/0256666 A1
(43) Pub. Date: Sep. 11, 2014

(54) ANTIBIOTIC CONJUGATES WITH NONSTEROIDAL ANTI-INFLAMMATORY DRUGS

(71) Applicant: Allergan, Inc., Irvine, CA (US)

(72) Inventors: Santosh C. Sinha, Ladera Ranch, CA (US); Smita S. Bhat, Irvine, CA (US); Ken Chow, Newport Coast, CA (US); LIMING WANG, Irvine, CA (US); MICHAEL E. GARST, Newport Beach, CA (US); MAYSSA ATTAR, Placentia, CA (US); BRANDON D. SWIFT, Camarillo, CA (US)

(73) Assignee: Allergan, Inc., Irvine, CA (US)

(21) Appl. No.: 14/198,881

(22) Filed: Mar. 6, 2014

Related U.S. Application Data
(60) Provisional application No. 61/775,199, filed on Mar. 8, 2013.

(51) Int. Cl. A61K 47/48

(52) U.S. Cl. CPC A61K 47/48115
USPC 514/40; 544/363; 514/253.08; 546/113; 514/300; 548/491; 514/419; 560/102; 514/534; 536/16.8

(57) ABSTRACT

The present invention describes single drug entities, formed by connecting an antibiotic moiety via a linker with a non-steroidal anti-inflammatory drug (NSAID) moiety. Upon topical application to the eye, the conjugate hybrid would undergo enzymatic and/or hydrolytic cleavage to release the individual antibiotic and NSAID drug.

### RELATED APPLICATIONS

This application claims the benefit of U.S. Provisional Patent Application Ser. No. 61/775,199 filed Mar. 8, 2013, the disclosure of which is hereby incorporated in its entirety by reference.

### FIELD OF THE INVENTION

The present invention describes single drug entities, formed by connecting an antibiotic moiety via a linker with a non-steroidal anti-inflammatory drug (NSAID) moiety. Upon topical application to the eye, the conjugate hybrid would undergo enzymatic and/or hydrolytic cleavage to release the individual antibiotic and NSAID drug.

### SUMMARY OF THE INVENTION

A conjugate drug, also referred to as a co-drug, a pro-drug, or a hybrid drug, comprises two or more different or same drugs within one single chemical entity wherein each drug contains an appropriate chemical functionality to enable them to be connected together, either directly or by means of a linker, which is a cleavable, bio-liable covalent linker.

Hybrid structures can incorporate two drugs joined together by a linker moiety such as an ester, a carboxylate, a carbonyl, a carbonate, an amido, a carbamate, a ketone, an amino, an oxo, an ethylene glycol, a polyethylene glycol, which is cleaved enzymatically or hydrolytically in vivo to release the active drugs.

The antibiotic moiety and the NSAID moiety, of the compounds disclosed herein are connected via two covalent bonds to a linker such that said compound degrades in vivo to yield the respective antibiotic and the respective NSAID. Each bond is an amide bond or an ester bond depending on the nature of the linker. In other words, the linker has one amide bond connecting to the antibiotic and one ester bond connecting to the NSAID. Alternatively, the linker is bonded to the antibiotic via ester bonds, or the linker is bonded to the antibiotic via amide bonds.

Degradation of the ester or amide bonds generally, but not necessarily, yields the corresponding acid and alcohol or amine by hydrolysis or a related reaction. A compound which degrades in vivo to yield the antibiotic and the NSAID, produces both active drugs belonging to distinct classes at some point in the metabolic process of the claimed compound. In many cases, cleavage of the first amide or ester bond will release one active, and cleavage of the second amide or ester bond will release the second active.

### BRIEF DESCRIPTION OF THE FIGURES

FIG. 1 Shows the cellular uptake of ester linked hybrid (parent) compounds and the hydrolyzed metabolites [non-steroidal anti-inflammatory (NSAID) and antibiotic] after a 2-hour incubation with Human Corneal Epithelial Cells.

### DETAILED DESCRIPTION OF THE INVENTION

In one aspect, the present invention relates to a compound comprising one antibiotic and one NSAID, or a pharmaceutical salt thereof, which are connected via two covalent bonds to a linker such that said compound degrades in vivo to yield the respective antibiotic and the respective non-steroidal anti-inflammatory drug, wherein each bond is an amide bond or an ester bond.

The hybrid compounds of the invention have both antibacterial and anti-inflammatory activities and are very useful compounds capable of producing the effect of an antibacterial drug and a non-steroidal anti-inflammatory drug in monotherapy.

In another aspect, the present invention relates to a compound which degrades in vivo into an antibiotic and a non-steroidal anti-inflammatory drug.

In another aspect, the present invention relates to a compound which comprises a linker having two bonds, wherein said bonds are asymmetrically degraded in vivo to release the two independent drugs: an antibiotic and a non-steroidal anti-inflammatory drug.

In another aspect, the present invention relates to a compound comprising one antibiotic and one non-steroidal anti-inflammatory drug or to a pharmaceutically acceptable salt thereof.
The hybrid drugs of the invention, provide a unique delivery of an antibiotic and a NSAID for the treatment of ophthalmic bacterial infections and for the prevention of inflammation. A single drug entity is advantageous to individual dosing of each drug because of the ability for simultaneous dosing and elimination of washout concerns when applying each drug separately.

The use of an antibiotic/NSAID hybrid drug is indicated where the risk of infection is high or where there is an expectation that potentially dangerous numbers of bacteria will be present in the eye. The anti-inflammatory component of the composition is useful in treating inflammation associated with physical trauma to ophthalmic tissues, inflammation associated with bacterial infections and inflammation resulting from surgical procedures. The combination of an antibiotic and NSAID is also useful in post-operative inflammation where there is an increased chance of bacterial infection. The composition of the invention may also be used prophylactically in connection with various ophthalmic surgical procedures that create a risk of bacterial infection. Other examples of ophthalmic conditions which may be treated with the compositions of the present invention include infective conditions associated with inflammation and where the use of NSAID is acceptable. Such conditions may include, but are not limited to conjunctivitis, keratitis, blepharitis, endophthalmitis, red eye, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, dry eye, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

The compounds disclosed herein comprise an antibiotic belonging to distinct classes:

Fluoroquinolones, which include, but are not limited to the following: levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, and sitafloxacin;

Cephalosporins, which include, but are not limited to the following: loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, and cephalothin;

Chloramphenicol;

Aminogycosides which include, but are not limited to, tobramycin, streptomycin, gentamicin, kanamycin, amikacin, netilmicin;

Penicillins, which include, but are not limited to, penicillin g, ticarcillin, methicillin, phenthicillin, cloxacillin, dicloxacillin, nafcillin, oxacillin;

Oxazolidinones, which include, but are not limited to linezolid.

Further, the compounds disclosed herein comprise a non-steroidal anti-inflammatory drug (NSAID) selected from: indomethacin, diclofenac, flurbiprofen, ketorolac, or suprofen.

In another embodiment the compounds disclosed herein comprise at least one antibiotic drug selected from levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin.

In another embodiment the compounds disclosed herein comprise at least one non-steroidal anti-inflammatory drug selected from: indomethacin, diclofenac, flurbiprofen, ketorolac, or suprofen.

Depending on the linking site, the hybrid compounds of the invention can be represented by Schemes 1:

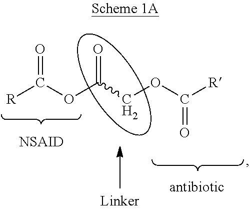

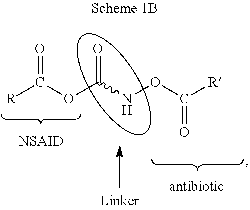

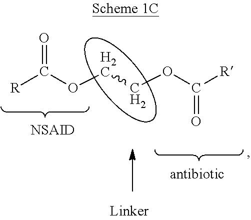

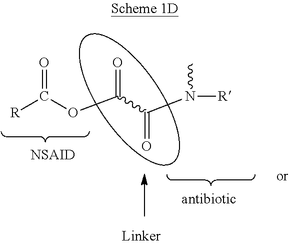

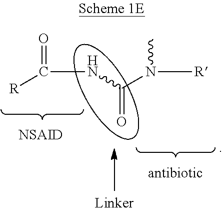

In another aspect the invention provides compounds which may comprise a linker moiety selected from, but not limited to, an ester, a carboxylate, a carbonyl, a carbonate, an amido, a carbamate, a ketone, an amino, an oxo, an ethylene glycol, a polyethylene glycol, an ethylene.

In another aspect, the invention provides compounds which may comprise a linker moiety comprising any combination of an ester, a carboxylate, a carbonyl, a carbonate, an amido, a carbamate, a ketone, an ethylene, an amino, an oxo, an ethylene glycol and/or a polyethylene glycol. Such linker moieties are exemplified below and linker structures are exemplified in Table 1.

Examples of ester moieties comprised in the linkers are:

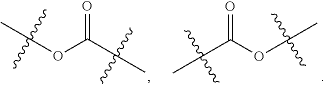 [SMILES: *OC(*)=O ; *OC(*)=O]

Examples of carboxylate moieties comprised in the linkers are:

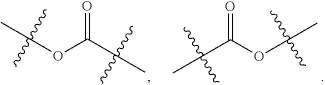 [SMILES: *OC(*)=O ; *OC(*)=O]

Example of a carbonyl moiety comprised in the linkers is

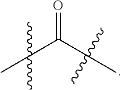 [SMILES: *C(*)=O]

Example of a carbonate moiety comprised in the linkers is:

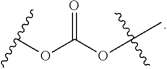 [SMILES: *OC(=O)O*]

Examples of amido moieties comprised in the linkers are:

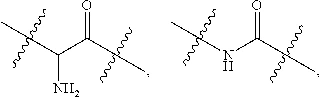 [SMILES: *C(=O)C(*)N ; *NC(*)=O]

Example of carbamate moiety comprised in the linkers is:

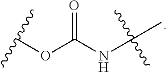 [SMILES: *NC(=O)O*]

Example of a ketone moiety comprised in the linkers is:

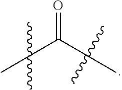 [SMILES: *C(*)=O]

Examples of amino moieties comprised in the linkers are:

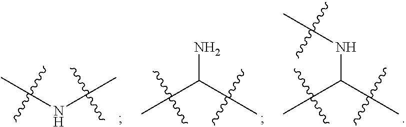

Example of an oxo moiety comprised in the linker is:

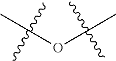 [SMILES: *O*]

Example of ethylene glycol moieties comprised in the linkers are:

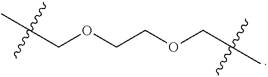 [SMILES: *COCCOC*]

Example of polyethylene glycol moiety comprised in the linkers is:

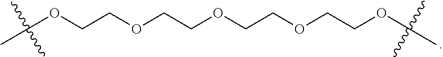 [SMILES: *OCCOCCOCCOCCO*]

Further the compounds disclosed herein comprise a linker selected from Table 1:

TABLE 1

| | Linker Number | | | | |
|---|---|---|---|---|---|
| Linker Structure | | n = 0 | n = 1 | n = 2 | n = 3 |
| 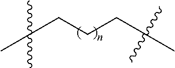 | | L2 | L1 | | |
| 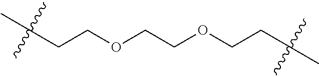 [SMILES: *CCOCCOCC*] | L3 | | | | |
| 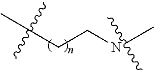 | | | L4 | | |
| 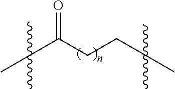 | | L35 | | L5 | |
| 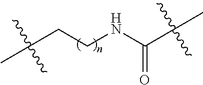 | | | L6 | | |
| 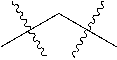 [SMILES: *C*] | L7 | | | | |
| 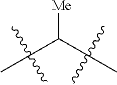 [SMILES: *C(*)C] | L14 | | | | |
| 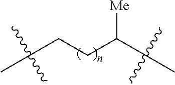 | | | L15 | | |
| 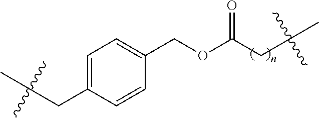 | | | L16 | | |
| 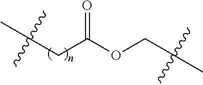 | | | L17 | | |
| 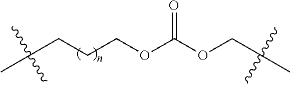 | | | L8 | | |
| 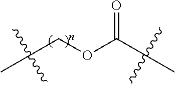 | | | L9 | | L43 |
| 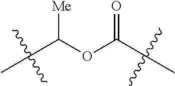 [SMILES: *C(=O)OC(*)C] | L10 | | | | |
| 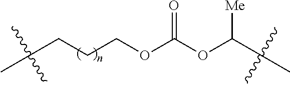 | | | L18 | | |
| 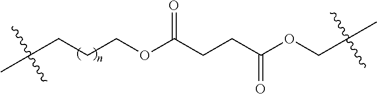 | | L48 | L11 | | |
| 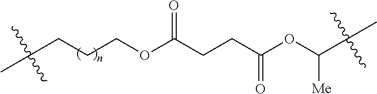 | | | L19 | | |
| 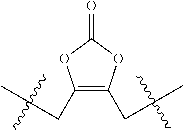 [SMILES: *Cc1oc(=O)oc1C*] | L12 | | | | |
| 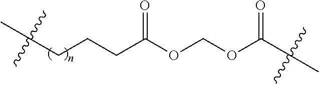 | | | L13 | | |
| 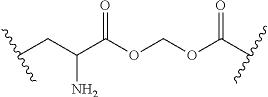 [SMILES: *CC(N)C(=O)OCOC(*)=O] | L20 | | | | |
| 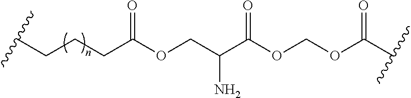 | | | L21 | | |
| 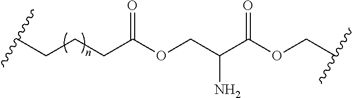 | | | L22 | | |
| 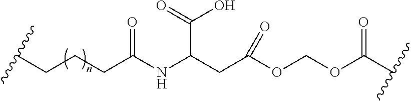 | | | L23 | | |
| 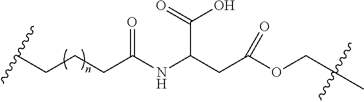 | | | L24 | | |
| 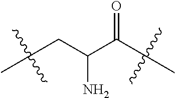 [SMILES: *CC(N)C(*)=O] | L25 | | | | |
| 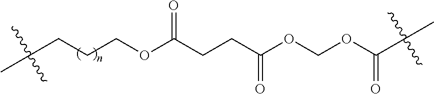 | | | L26 | | |
| 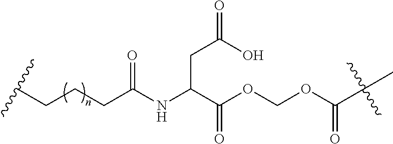 | | | L27 | | |
| 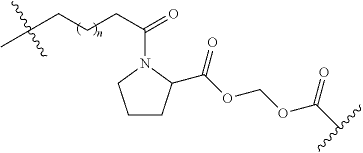 | | | L28 | | |
| 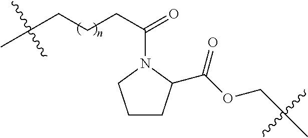 | | | L29 | | |
| 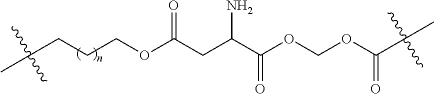 | | | L30 | | |
| 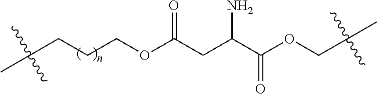 | | | L31 | | |
| 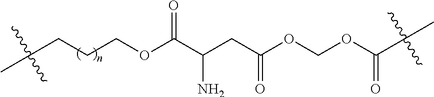 | | | L32 | | |
| 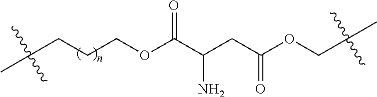 | | | L33 | | |
| 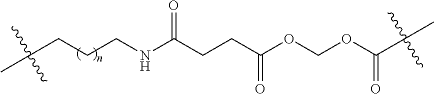 | | | L34 | | |
| 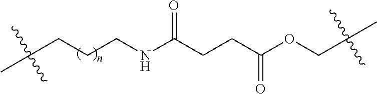 | | | L35 | | |
| 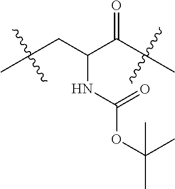 [SMILES: *CC(NC(=O)OC(C)(C)C)C(*)=O] | L36 | | | | |
| 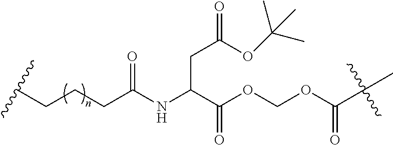 | | | L37 | | |
| 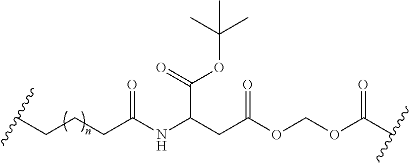 | | | L38 | | |
| 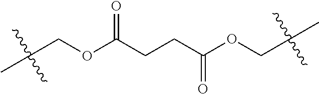 [SMILES: *COC(=O)CCC(=O)OC*] | L39 | | | | |
| 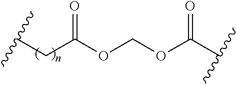 | | | L40 | | |
| 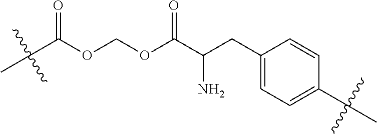 [SMILES: *C(=O)OCOC(=O)C(N)Cc1ccc(*)cc1] | L42 | | | | |
| 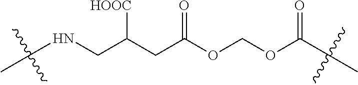 [SMILES: *NCC(CC(=O)OCOC(*)=O)C(=O)O] | L44 | | | | |
| 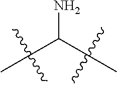 [SMILES: *C(*)N] | L41 | | | | |
| 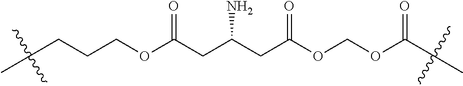 [SMILES: *CCCOC(=O)C[C@H](N)CC(=O)OCOC(*)=O] | L45 | | | | |
| 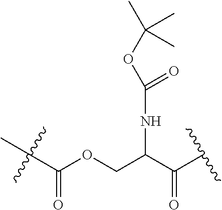 [SMILES: *C(=O)OCC(NC(=O)OC(C)(C)C)C(*)=O] | L46 | | | | |
| 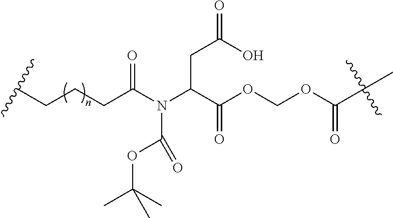 | | | L47 | | |
| 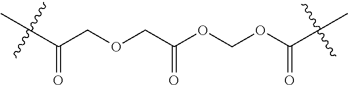 [SMILES: *C(=O)COCC(=O)OCOC(*)=O] | L49 | | | | |
| 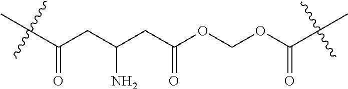 [SMILES: *C(=O)CC(N)CC(=O)OCOC(*)=O] | L50 | | | | |
| 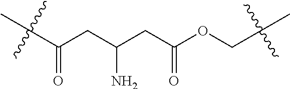 [SMILES: *COC(=O)CC(N)CC(*)=O] | L51 | | | | |
| 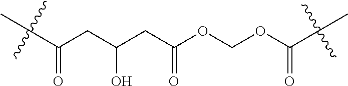 [SMILES: *C(=O)CC(O)CC(=O)OCOC(*)=O] | L52 | | | | |
| 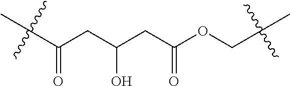 [SMILES: *COC(=O)CC(O)CC(*)=O] | L53 | | | | |
| 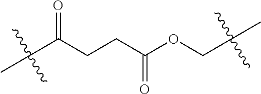 [SMILES: *COC(=O)CCC(*)=O] | L54 | | | | |
| 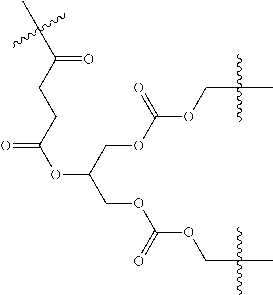 [SMILES: *COC(=O)OCC(COC(=O)OC*)OC(=O)CCC(*)=O] | L55 | | | | |
| 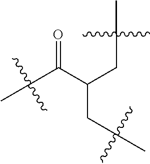 [SMILES: *CC(C*)C(*)=O] | L56 | | | | |
| 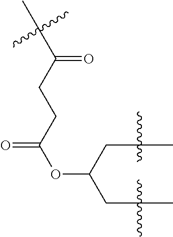 [SMILES: *CC(C*)OC(=O)CCC(*)=O] | L57 | | | | |
| 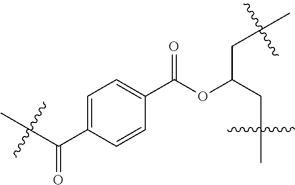 [SMILES: *CC(C*)OC(=O)c1ccc(C(*)=O)cc1] | L58 | | | | |
| 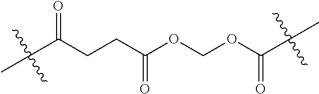 [SMILES: *C(=O)CCC(=O)OCOC(*)=O] | L59 | | | | |
| 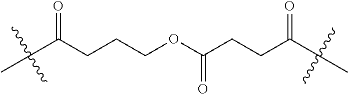 [SMILES: *C(=O)CCCOC(=O)CCC(*)=O] | L60 | | | | |
| 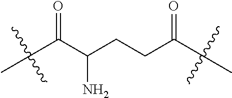 [SMILES: *C(=O)CCC(N)C(*)=O] | L61 | | | | |
| 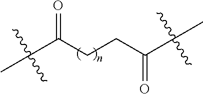 | | | L63 | | |
| 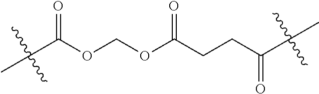 [SMILES: *C(=O)CCC(=O)OCOC(*)=O] | L64 | | | | |
| 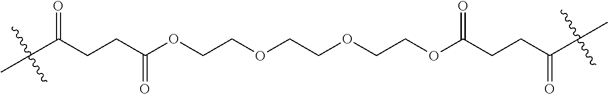 [SMILES: *C(=O)CCC(=O)OCCOCCOCCOC(=O)CCC(*)=O] | L66 | | | | |
| 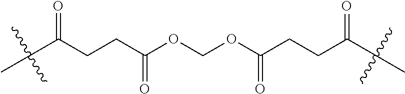 [SMILES: *C(=O)CCC(=O)OCOC(=O)CCC(*)=O] | L67 | | | | |
| 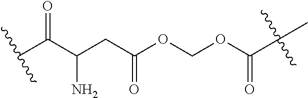 [SMILES: *C(=O)OCOC(=O)CC(N)C(*)=O] | L68 | | | | |
| 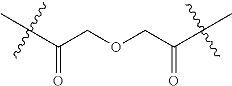 [SMILES: *C(=O)COCC(*)=O] | L69 | | | | |
| 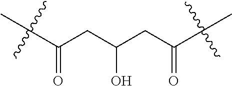 [SMILES: *C(=O)CC(O)CC(*)=O] | L70 | | | | |
| 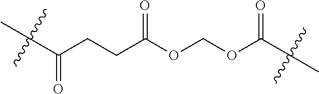 [SMILES: *C(=O)CCC(=O)OCOC(*)=O] | L71 | | | | |
| 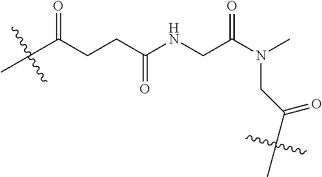 [SMILES: *C(=O)CCC(=O)NCC(=O)N(C)CC(*)=O] | L72 | | | | |
| 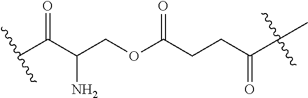 [SMILES: *C(=O)CCC(=O)OCC(N)C(*)=O] | L73 | | | | |
| 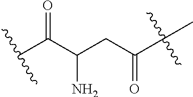 [SMILES: *C(=O)CC(N)C(*)=O] | L74 | | | | |
| 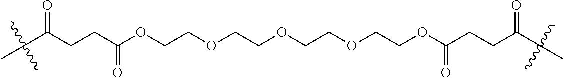 [SMILES: *C(=O)CCC(=O)OCCOCCOCCOCCOC(=O)CCC(*)=O] | L75 | | | | |
| 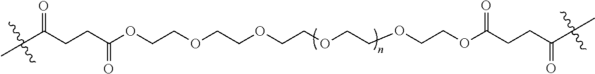 | | | L76 | L104 | L105 |
| 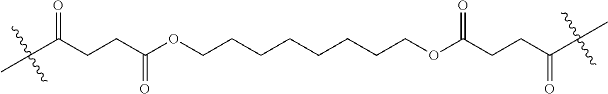 [SMILES: *C(=O)CCC(=O)OCCCCCCCCOC(=O)CCC(*)=O] | L77 | | | | |
| 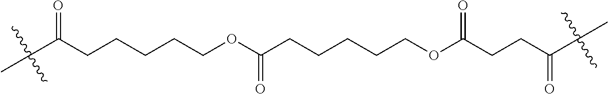 [SMILES: *C(=O)CCCCCOC(=O)CCCCCOC(=O)CCC(*)=O] | L78 | | | | |
| 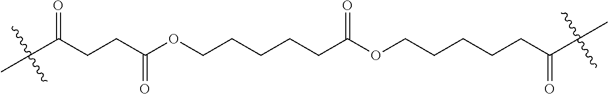 [SMILES: *C(=O)CCCCCOC(=O)CCCCCOC(=O)CCC(*)=O] | L79 | | | | |
| 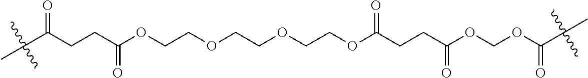 [SMILES: *C(=O)CCC(=O)OCCOCCOCCOC(=O)CCC(=O)OCOC(*)=O] | L78 | | | | |
| 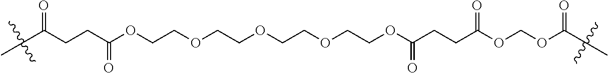 [SMILES: *C(=O)CCC(=O)OCCOCCOCCOCCOC(=O)CCC(=O)OCOC(*)=O] | L80 | | | | |
| 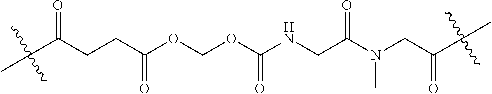 [SMILES: *C(=O)CCC(=O)OCOC(=O)NCC(=O)N(C)CC(*)=O] | L81 | | | | |
| 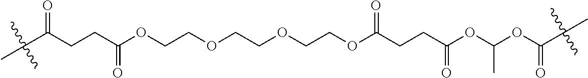 [SMILES: *C(=O)CCC(=O)OCCOCCOCCOC(=O)CCC(=O)OC(C)OC(*)=O] | L82 | | | | |
| 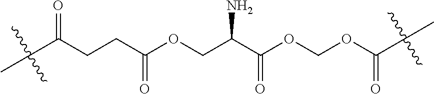 [SMILES: *C(=O)CCC(=O)OC[C@@H](N)C(=O)OCOC(*)=O] | L83 | | | | |
| 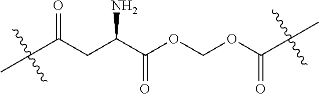 [SMILES: *C(=O)C[C@@H](N)C(=O)OCOC(*)=O] | L84 | | | | |
| 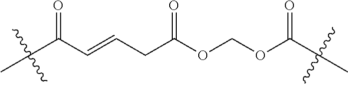 [SMILES: *C(=O)/C=C/CC(=O)OCOC(*)=O] | L85 | | | | |
| 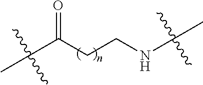 | | | L86 | L87 | |
| 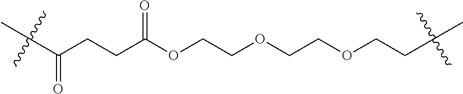 [SMILES: *CCOCCOCCOC(=O)CCC(*)=O] | L88 | | | | |
| 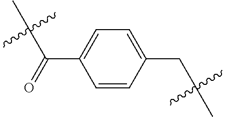 [SMILES: *Cc1ccc(C(*)=O)cc1] | L89 | | | | |
| 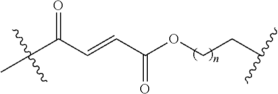 | | | L90 | | |
| 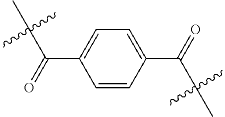 [SMILES: *C(=O)c1ccc(C(*)=O)cc1] | L91 | | | | |
| 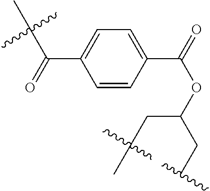 [SMILES: *CC(C*)OC(=O)c1ccc(C(*)=O)cc1] | L92 | | | | |
| 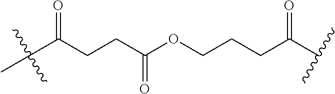 [SMILES: *C(=O)CCCOC(=O)CCC(*)=O] | L93 | | | | |
| 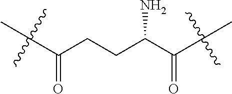 [SMILES: *C(=O)CC[C@H](N)C(*)=O] | L94 | | | | |
| 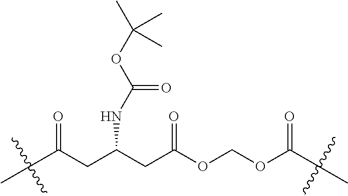 [SMILES: *C(=O)C[C@@H](CC(=O)OCOC(*)=O)NC(=O)OC(C)(C)C] | L95 | | | | |
| 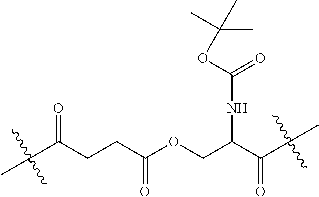 [SMILES: *C(=O)CCC(=O)OCC(NC(=O)OC(C)(C)C)C(*)=O] | L96 | | | | |
| 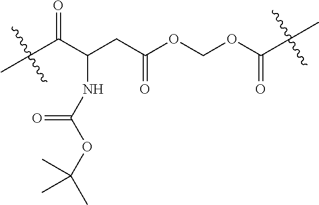 [SMILES: *C(=O)OCOC(=O)CC(NC(=O)OC(C)(C)C)C(*)=O] | L97 | | | | |
| 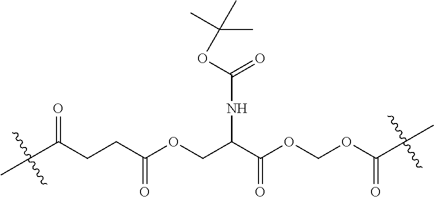 [SMILES: *C(=O)CCC(=O)OCC(NC(=O)OC(C)(C)C)C(=O)OCOC(*)=O] | L98 | | | | |
| 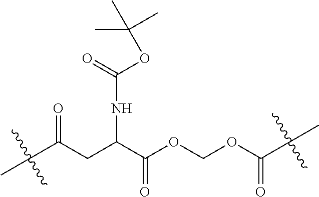 [SMILES: *C(=O)CC(NC(=O)OC(C)(C)C)C(=O)OCOC(*)=O] | L99 | | | | |
| 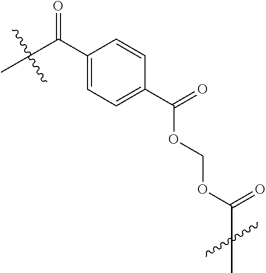 [SMILES: *C(=O)OCOC(=O)c1ccc(C(*)=O)cc1] | L100 | | | | |
| 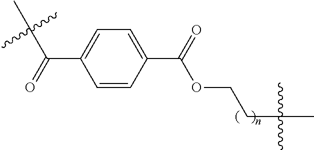 | | L101 | L102 | L103 | |

Further the compounds disclosed herein comprise a pro-drug moiety selected from Table 2:

TABLE 2

| Pro-drug Structure | Pro-drug Number |
|---|---|
| 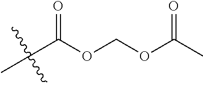 [SMILES: *C(=O)OCOC(C)=O] | P1 |
| 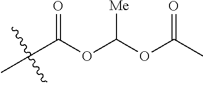 [SMILES: *C(=O)OC(C)OC(C)=O] | P2 |
| 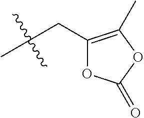 [SMILES: *Cc1oc(=O)oc1C] | P3 |
| 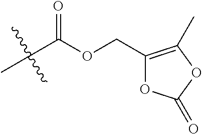 [SMILES: *C(=O)OCc1oc(=O)oc1C] | P4 |
| 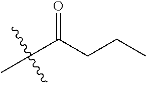 [SMILES: *C(=O)CCC] | P5 |
| 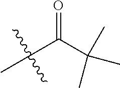 [SMILES: *C(=O)C(C)(C)C] | P6 |
| 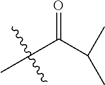 [SMILES: *C(=O)C(C)C] | P7 |
| 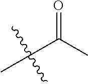 [SMILES: *C(C)=O] | P8 |
| 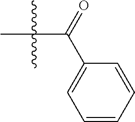 [SMILES: *C(=O)c1ccccc1] | P9 |
| 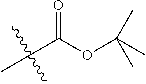 [SMILES: *C(=O)OC(C)(C)C] | P10 |
| 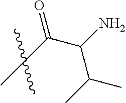 [SMILES: *C(=O)C(N)C(C)C] | P11 |
| 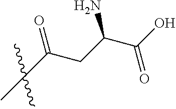 [SMILES: *C(=O)C[C@@H](N)C(=O)O] | P12 |
| 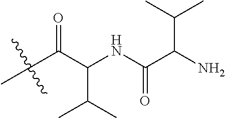 [SMILES: *C(=O)C(NC(=O)C(N)C(C)C)C(C)C] | P13 |
| 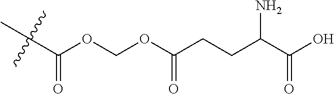 [SMILES: *C(=O)OCOC(=O)CCC(N)C(=O)O] | P14 |
| 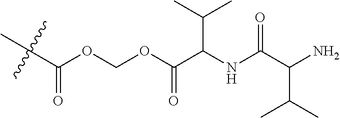 [SMILES: *C(=O)OCOC(=O)C(NC(=O)C(N)C(C)C)C(C)C] | P15 |

Compounds of the invention are shown in Table 3:

TABLE 3

| Compound | |
|---|---|
| No | IUPAC Name |
| 77 | 7-[4-({1-[(4-chlorophenyl)carbonyl]-5-methoxy-2-methyl-1H-indol- |
| | 3-yl}acetyl)-3-methylpiperazin-1-yl]-1-cyclopropyl-6-fluoro-8- |
| | methoxy-4-oxo-1,4-dihydroquinoline-3-carboxylic acid |
| 44 | 2-{2-[2-({[5-(phenylcarbonyl)-2,3-dihydro-1H-pyrrolizin-1- |
| | yl]carbonyl}oxy)ethoxy]ethoxy}ethyl 1-cyclopropyl-6-fluoro-8- |
| | methoxy-7-(3-methylpiperazin-1-yl)-4-oxo-1,4-dihydroquinoline-3- |
| | carboxylate |
| 30 | 2-(2-{2-[({1-[(4-chlorophenyl)carbonyl]-5-methoxy-2-methyl-1H- |
| | indol-3-yl}acetyl)oxy]ethoxy}ethoxy)ethyl 1-cyclopropyl-6-fluoro-8- |
| | methoxy-7-(3-methylpiperazin-1-yl)-4-oxo-1,4-dihydroquinoline-3- |
| | carboxylate |
| 65 | 2-{2-[2-({[5-(phenylcarbonyl)-2,3-dihydro-1H-pyrrolizin-1- |
| | yl]carbonyl}oxy)ethoxy]ethoxy}ethyl rel-1-cyclopropyl-7- |
| | [(4aR,7aR)-4a,7a-dimethyloctahydro-6H-pyrrolo[3,4-b]pyridin-6- |
| | yl]-6-fluoro-8-methoxy-4-oxo-1,4-dihydroquinoline-3-carboxylate |
| 59 | 2-(2-{2-[({1-[(4-chlorophenyl)carbonyl]-5-methoxy-2-methyl-1H- |
| | indol-3-yl}acetyl)oxy]ethoxy}ethoxy)ethyl rel-1-cyclopropyl-7- |
| | [(4aR,7aR)-4a,7a-dimethyloctahydro-6H-pyrrolo[3,4-b]pyridin-6- |
| | yl]-6-fluoro-8-methoxy-4-oxo-1,4-dihydroquinoline-3-carboxylate |
| 45 | 2-({[1-cyclopropyl-6-fluoro-8-methoxy-7-(3-methylpiperazin-1-yl)- |
| | 4-oxo-1,4-dihydroquinolin-3-yl]carbonyl}amino)ethyl 5- |
| | (phenylcarbonyl)-2,3-dihydro-1H-pyrrolizine-1-carboxylate |
| 66 | 2-[({1-cyclopropyl-7-[(4aR,7aR)-4a,7a-dimethyloctahydro-6H- |
| | pyrrolo[3,4-b]pyridin-6-yl]-6-fluoro-8-methoxy-4-oxo-1,4- |
| | dihydroquinolin-3-yl}carbonyl)amino]ethyl rel-5-(phenylcarbonyl)- |
| | 2,3-dihydro-1H-pyrrolizine-1-carboxylate |
| 31 | 3-[({1-[(4-chlorophenyl)carbonyl]-5-methoxy-2-methyl-1H-indol-3- |
| | yl}acetyl)oxy]propyl 1-cyclopropyl-6-fluoro-8-methoxy-7-(3- |
| | methylpiperazin-1-yl)-4-oxo-1,4-dihydroquinoline-3-carboxylate |
| 1 | 2-[2-(2-{[2-(2-fluorobiphenyl-4- |
| | yl)propanoyl]oxy}ethoxy)ethoxy]ethyl 1-cyclopropyl-6-fluoro-8- |
| | methoxy-7-(3-methylpiperazin-1-yl)-4-oxo-1,4-dihydroquinoline-3- |
| | carboxylate |
| 76 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro- |
| | 2H-pyran-2-yl]oxy}-2-hydroxycyclohexyl]oxy}-3,5- |
| | dihydroxytetrahydro-2H-pyran-2-yl]methyl rel-4-{[2-(2- |
| | fluorobiphenyl-4-yl)propanoyl]oxy}butanoate |
| 70 | 2-[({2-[(dichloroacetyl)amino]-3-hydroxy-3-(4- |
| | nitrophenyl)propoxy}carbonyl)amino]ethyl {1-[(4- |
| | chlorophenyl)carbonyl]-5-methoxy-2-methyl-1H-indol-3-yl}acetate |
| 75 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro- |
| | 2H-pyran-2-yl]oxy}-2-hydroxycyclohexyl]oxy}-3,5- |
| | dihydroxytetrahydro-2H-pyran-2-yl]methyl rel-4-[({1-[(4- |
| | chlorophenyl)carbonyl]-5-methoxy-2-methyl-1H-indol-3- |
| | yl}acetyl)oxy]butanoate |
| 2 | 3-{[2-(2-fluorobiphenyl-4-yl)propanoyl]oxy}propyl 1-cyclopropyl-6- |
| | fluoro-8-methoxy-7-(3-methylpiperazin-1-yl)-4-oxo-1,4- |
| | dihydroquinoline-3-carboxylate |
| 3 | 2-{[2-(2-fluorobiphenyl-4-yl)propanoyl]oxy}ethyl 1-cyclopropyl-6- |
| | fluoro-8-methoxy-7-(3-methylpiperazin-1-yl)-4-oxo-1,4- |
| | dihydroquinoline-3-carboxylate |
| 55 | 2-{[2-(2-fluorobiphenyl-4-yl)propanoyl]oxy}ethyl 1-cyclopropyl-6- |
| | fluoro-8-methoxy-7-(octahydro-6H-pyrrolo[3,4-b]pyridin-6-yl)-4- |
| | oxo-1,4-dihydroquinoline-3-carboxylate |
| 60 | 3-[({1-[(4-chlorophenyl)carbonyl]-5-methoxy-2-methyl-1H-indol-3- |
| | yl}acetyl)oxy]propyl rel-1-cyclopropyl-6-fluoro-8-methoxy-7- |
| | [(4aR,7aR)-octahydro-6H-pyrrolo[3,4-b]pyridin-6-yl]-4-oxo-1,4- |
| | dihydroquinoline-3-carboxylate |
| 41 | 3-[({2-[(2,6-dichlorophenyl)amino]phenyl}acetyl)oxy]propyl 1- |
| | cyclopropyl-6-fluoro-8-methoxy-7-(3-methylpiperazin-1-yl)-4-oxo- |
| | 1,4-dihydroquinoline-3-carboxylate |
| 64 | 3-[({2-[(2,6-dichlorophenyl)amino]phenyl}acetyl)oxy]propyl rel-1- |
| | cyclopropyl-6-fluoro-8-methoxy-7-[(4aR,7aR)-octahydro-6H- |
| | pyrrolo[3,4-b]pyridin-6-yl]-4-oxo-1,4-dihydroquinoline-3- |
| | carboxylate |
| 71 | 2-[(dichloroacetyl)amino]-3-hydroxy-3-(4-nitrophenyl)propyl 4-[({1- |
| | [(4-chlorophenyl)carbonyl]-5-methoxy-2-methyl-1H-indol-3- |
| | yl}acetyl)oxy]butanoate |
| 74 | 2-[(dichloroacetyl)amino]-3-hydroxy-1-(4-nitrophenyl)propyl 4-{[2- |
| | (2-fluorobiphenyl-4-yl)propanoyl]oxy}butanoate |
| 72 | 2-[(dichloroacetyl)amino]-3-hydroxy-3-(4-nitrophenyl)propyl {1-[(4- |
| | chlorophenyl)carbonyl]-5-methoxy-2-methyl-1H-indol-3-yl}acetate |
| 48 | 1-cyclopropyl-6-fluoro-8-methoxy-7-(3-methyl-4-{[({[5- |
| | (phenylcarbonyl)-2,3-dihydro-1H-pyrrolizin-1- |
| | yl]carbonyl}oxy)methoxy]carbonyl}piperazin-1-yl)-4-oxo-1,4- |
| | dihydroquinoline-3-carboxylic acid |
| 38 | 7-[4-({[({1-[(4-chlorophenyl)carbonyl]-5-methoxy-2-methyl-1H- |
| | indol-3-yl}acetyl)oxy]methoxy}carbonyl)-3-methylpiperazin-1-yl]-1- |
| | cyclopropyl-6-fluoro-8-methoxy-4-oxo-1,4-dihydroquinoline-3- |
| | carboxylic acid |
| 67 | rel-1-cyclopropyl-6-fluoro-8-methoxy-4-oxo-7-[(4aR,7aR)-1-{[({[5- |
| | (phenylcarbonyl)-2,3-dihydro-1H-pyrrolizin-1- |
| | yl]carbonyl}oxy)methoxy]carbonyl}octahydro-6H-pyrrolo[3,4- |
| | b]pyridin-6-yl]-1,4-dihydroquinoline-3-carboxylic acid |
| 4 | 3-{[2-(2-fluorobiphenyl-4-yl)propanoyl]oxy}propyl 1-cyclopropyl-6- |
| | fluoro-8-methoxy-7-{3-methyl-4-[(5-methyl-2-oxo-1,3-dioxol-4- |
| | yl)methyl]piperazin-1-yl}-4-oxo-1,4-dihydroquinoline-3- |
| | carboxylate |
| 32 | [({1-[(4-chlorophenyl)carbonyl]-5-methoxy-2-methyl-1H-indol-3- |
| | yl}acetyl)oxy]methyl 1-cyclopropyl-6-fluoro-8-methoxy-7-(3- |
| | methylpiperazin-1-yl)-4-oxo-1,4-dihydroquinoline-3-carboxylate |
| 46 | ({[5-(phenylcarbonyl)-2,3-dihydro-1H-pyrrolizin-1- |
| | yl]carbonyl}oxy)methyl 1-cyclopropyl-6-fluoro-8-methoxy-7-(3- |
| | methylpiperazin-1-yl)-4-oxo-1,4-dihydroquinoline-3-carboxylate |
| 33 | 2-[({1-[(4-chlorophenyl)carbonyl]-5-methoxy-2-methyl-1H-indol-3- |
| | yl}acetyl)oxy]ethyl 1-cyclopropyl-6-fluoro-8-methoxy-7-{3-methyl- |
| | 4-[(5-methyl-2-oxo-1,3-dioxol-4-yl)methyl]piperazin-1-yl}-4-oxo- |
| | 1,4-dihydroquinoline-3-carboxylate |
| 34 | 3-[({1-[(4-chlorophenyl)carbonyl]-5-methoxy-2-methyl-1H-indol-3- |
| | yl}acetyl)oxy]propyl 1-cyclopropyl-6-fluoro-8-methoxy-7-{3- |
| | methyl-4-[(5-methyl-2-oxo-1,3-dioxol-4-yl)methyl]piperazin-1-yl}- |
| | 4-oxo-1,4-dihydroquinoline-3-carboxylate |
| 5 | 2-{[2-(2-fluorobiphenyl-4-yl)propanoyl]oxy}ethyl 1-cyclopropyl-6- |
| | fluoro-8-methoxy-7-{3-methyl-4-[(5-methyl-2-oxo-1,3-dioxol-4- |
| | yl)methyl]piperazin-1-yl}-4-oxo-1,4-dihydroquinoline-3- |
| | carboxylate |
| 61 | [({1-[(4-chlorophenyl)carbonyl]-5-methoxy-2-methyl-1H-indol-3- |
| | yl}acetyl)oxy]methyl rel-1-cyclopropyl-6-fluoro-8-methoxy-7- |
| | [(4aR,7aR)-octahydro-6H-pyrrolo[3,4-b]pyridin-6-yl]-4-oxo-1,4- |
| | dihydroquinoline-3-carboxylate |
| 35 | [({3-[({1-[(4-chlorophenyl)carbonyl]-5-methoxy-2-methyl-1H-indol- |
| | 3-yl}acetyl)oxy]propoxy}carbonyl)oxy]methyl 1-cyclopropyl-6- |
| | fluoro-8-methoxy-7-(3-methylpiperazin-1-yl)-4-oxo-1,4- |
| | dihydroquinoline-3-carboxylate |
| 6 | 2-[2-(2-{[2-(2-fluorobiphenyl-4- |
| | yl)propanoyl]oxy}ethoxy)ethoxy]ethyl 1-cyclopropyl-6-fluoro-8- |
| | methoxy-7-{3-methyl-4-[(5-methyl-2-oxo-1,3-dioxol-4- |
| | yl)methyl]piperazin-1-yl}-4-oxo-1,4-dihydroquinoline-3- |
| | carboxylate |
| 36 | 2-(2-{2-[({1-[(4-chlorophenyl)carbonyl]-5-methoxy-2-methyl-1H- |
| | indol-3-yl}acetyl)oxy]ethoxy}ethoxy)ethyl 1-cyclopropyl-6-fluoro-8- |
| | methoxy-7-{3-methyl-4-[(5-methyl-2-oxo-1,3-dioxol-4- |
| | yl)methyl]piperazin-1-yl}-4-oxo-1,4-dihydroquinoline-3- |
| | carboxylate |
| 37 | 3-[({1-[(4-chlorophenyl)carbonyl]-5-methoxy-2-methyl-1H-indol-3- |
| | yl}acetyl)oxy]propyl ({[1-cyclopropyl-6-fluoro-8-methoxy-7-(3- |
| | methylpiperazin-1-yl)-4-oxo-1,4-dihydroquinolin-3- |
| | yl]carbonyl}oxy)methyl butanedioate |
| 8 | ({[1-cyclopropyl-6-fluoro-8-methoxy-7-(3-methylpiperazin-1-yl)-4- |
| | oxo-1,4-dihydroquinolin-3-yl]carbonyl}oxy)methyl 3-{[2-(2- |
| | fluorobiphenyl-4-yl)propanoyl]oxy}propyl butanedioate |
| 7 | {[(3-{[2-(2-fluorobiphenyl-4- |
| | yl)propanoyl]oxy}propoxy)carbonyl]oxy}methyl 1-cyclopropyl-6- |
| | fluoro-8-methoxy-7-(3-methylpiperazin-1-yl)-4-oxo-1,4- |
| | dihydroquinoline-3-carboxylate |
| 9 | [5-({[2-(2-fluorobiphenyl-4-yl)propanoyl]oxy}methyl)-2-oxo-1,3- |
| | dioxol-4-yl]methyl 1-cyclopropyl-6-fluoro-8-methoxy-7-(3- |
| | methylpiperazin-1-yl)-4-oxo-1,4-dihydroquinoline-3-carboxylate |
| 15 | 1-cyclopropyl-6-fluoro-7-(4-{[5-({[2-(2-fluorobiphenyl-4- |
| | yl)propanoyl]oxy}methyl)-2-oxo-1,3-dioxol-4-yl]methyl}-3- |
| | methylpiperazin-1-yl)-8-methoxy-4-oxo-1,4-dihydroquinoline-3- |
| | carboxylic acid |
| 10 | {[2-(2-fluorobiphenyl-4-yl)propanoyl]oxy}methyl 1-cyclopropyl-6- |
| | fluoro-8-methoxy-7-(3-methylpiperazin-1-yl)-4-oxo-1,4- |
| | dihydroquinoline-3-carboxylate |
| 16 | 1-cyclopropyl-6-fluoro-7-{4-[({[2-(2-fluorobiphenyl-4- |
| | yl)propanoyl]oxy}methoxy)carbonyl]-3-methylpiperazin-1-yl}-8- |
| | methoxy-4-oxo-1,4-dihydroquinoline-3-carboxylic acid |
| 39 | 7-(4-{[({[({1-[(4-chlorophenyl)carbonyl]-5-methoxy-2-methyl-1H- |
| | indol-3-yl}acetyl)oxy]acetyl}oxy)methoxy]carbonyl}-3- |
| | methylpiperazin-1-yl)-1-cyclopropyl-6-fluoro-8-methoxy-4-oxo- |
| | 1,4-dihydroquinoline-3-carboxylic acid |
| 43 | 1-cyclopropyl-7-[4-({[({2-[(2,6- |
| | dichlorophenyl)amino]phenyl}acetyl)oxy]methoxy}carbonyl)-3- |
| | methylpiperazin-1-yl]-6-fluoro-8-methoxy-4-oxo-1,4- |
| | dihydroquinoline-3-carboxylic acid |
| 42 | [({2-[(2,6-dichlorophenyl)amino]phenyl}acetyl)oxy]methyl 1- |
| | cyclopropyl-6-fluoro-8-methoxy-7-(3-methylpiperazin-1-yl)-4-oxo- |
| | 1,4-dihydroquinoline-3-carboxylate |
| 11 | [(4-{[2-(2-fluorobiphenyl-4-yl)propanoyl]oxy}butanoyl)oxy]methyl |
| | 1-cyclopropyl-6-fluoro-8-methoxy-7-(3-methylpiperazin-1-yl)-4- |
| | oxo-1,4-dihydroquinoline-3-carboxylate |
| 17 | 1-cyclopropyl-6-fluoro-7-[4-({[(4-{[2-(2-fluorobiphenyl-4- |
| | yl)propanoyl]oxy}butanoyl)oxy]methoxy}carbonyl)-3- |
| | methylpiperazin-1-yl]-8-methoxy-4-oxo-1,4-dihydroquinoline-3- |
| | carboxylic acid |
| 73 | 2-{2-[(dichloroacetyl)amino]-3-hydroxy-3-(4-nitrophenyl)propoxy}- |
| | 2-oxoethyl {1-[(4-chlorophenyl)carbonyl]-5-methoxy-2-methyl-1H- |
| | indol-3-yl}acetate |
| 53 | 1-cyclopropyl-6-fluoro-8-methoxy-7-(3-methyl-4-{[({2-[4-(thiophen- |
| | 2-ylcarbonyl)phenyl]propanoyl}oxy)methoxy]carbonyl}piperazin-1- |
| | yl)-4-oxo-1,4-dihydroquinoline-3-carboxylic acid |
| 51 | ({2-[4-(thiophen-2-ylcarbonyl)phenyl]propanoyl}oxy)methyl 1- |
| | cyclopropyl-6-fluoro-8-methoxy-7-(3-methylpiperazin-1-yl)-4-oxo- |
| | 1,4-dihydroquinoline-3-carboxylate |
| 12 | ({[1-cyclopropyl-6-fluoro-8-methoxy-7-(3-methylpiperazin-1-yl)-4- |
| | oxo-1,4-dihydroquinolin-3-yl]carbonyl}oxy)methyl {[2-(2- |
| | fluorobiphenyl-4-yl)propanoyl]oxy}methyl butanedioate |
| 56 | 1-cyclopropyl-6-fluoro-7-{1-[({[2-(2-fluorobiphenyl-4- |
| | yl)propanoyl]oxy}methoxy)carbonyl]octahydro-6H-pyrrolo[3,4- |
| | b]pyridin-6-yl}-8-methoxy-4-oxo-1,4-dihydroquinoline-3-carboxylic |
| | acid |
| 62 | 7-[1-({[({1-[(4-chlorophenyl)carbonyl]-5-methoxy-2-methyl-1H- |
| | inden-3-yl}acetyl)oxy]methoxy}carbonyl)octahydro-6H- |
| | pyrrolo[3,4-b]pyridin-6-yl]-1-cyclopropyl-6-fluoro-8-methoxy-4- |
| | oxo-1,4-dihydroquinoline-3-carboxylic acid |
| 57 | 1-cyclopropyl-6-fluoro-7-[1-({[(4-{[2-(2-fluorobiphenyl-4- |
| | yl)propanoyl]oxy}butanoyl)oxy]methoxy}carbonyl)octahydro-6H- |
| | pyrrolo[3,4-b]pyridin-6-yl]-8-methoxy-4-oxo-1,4-dihydroquinoline- |
| | 3-carboxylic acid |
| 63 | 7-(1-{[({4-[({1-[(4-chlorophenyl)carbonyl]-5-methoxy-2-methyl-1H- |
| | inden-3-yl}acetyl)oxy]butanoyl}oxy)methoxy]carbonyl}octahydro- |
| | 6H-pyrrolo[3,4-b]pyridin-6-yl)-1-cyclopropyl-6-fluoro-8-methoxy-4- |
| | oxo-1,4-dihydroquinoline-3-carboxylic acid |
| 40 | 7-(4-{[({4-[({1-[(4-chlorophenyl)carbonyl]-5-methoxy-2-methyl-1H- |
| | inden-3-yl}acetyl)oxy]butanoyl}oxy)methoxy]carbonyl}-3- |
| | methylpiperazin-1-yl)-1-cyclopropyl-6-fluoro-8-methoxy-4-oxo- |
| | 1,4-dihydroquinoline-3-carboxylic acid |
| 68 | 1-cyclopropyl-6-fluoro-8-methoxy-4-oxo-7-{1-[({[4-({[5- |
| | (phenylcarbonyl)-2,3-dihydro-1H-pyrrolizin-1- |
| | yl]carbonyl}oxy)butanoyl]oxy}methoxy)carbonyl]octahydro-6H- |
| | pyrrolo[3,4-b]pyridin-6-yl}-1,4-dihydroquinoline-3-carboxylic acid |
| 49 | 1-cyclopropyl-6-fluoro-8-methoxy-7-{3-methyl-4-[({[4-({[5- |
| | (phenylcarbonyl)-2,3-dihydro-1H-pyrrolizin-1- |
| | yl]carbonyl}oxy)butanoyl]oxy}methoxy)carbonyl]piperazin-1-yl}-4- |
| | oxo-1,4-dihydroquinoline-3-carboxylic acid |
| 80 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro- |
| | 2H-pyran-2-yl]oxy}-2-hydroxycyclohexyl]oxy}-3,5- |
| | dihydroxytetrahydro-2H-pyran-2-yl]methyl rel-2-(2-fluorobiphenyl- |
| | 4-yl)propanoate |
| 78 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro- |
| | 2H-pyran-2-yl]oxy}-2-hydroxycyclohexyl]oxy}-3,5- |
| | dihydroxytetrahydro-2H-pyran-2-yl]methyl rel-{1-[(4- |
| | chlorophenyl)carbonyl]-5-methoxy-2-methyl-1H-indol-3-yl}acetate |
| 18 | 1-cyclopropyl-6-fluoro-7-{4-[15-(2-fluorobiphenyl-4-yl)-5,8,14- |
| | trioxo-2,4,9,13-tetraoxahexadecan-1-oyl]-3-methylpiperazin-1-yl}- |
| | 8-methoxy-4-oxo-1,4-dihydroquinoline-3-carboxylic acid |
| 58 | 1-cyclopropyl-6-fluoro-7-{1-[15-(2-fluorobiphenyl-4-yl)-5,8,14- |
| | trioxo-2,4,9,13-tetraoxahexadecan-1-oyl]octahydro-6H- |
| | pyrrolo[3,4-b]pyridin-6-yl}-8-methoxy-4-oxo-1,4-dihydroquinoline- |
| | 3-carboxylic acid |
| 54 | 1-cyclopropyl-6-fluoro-8-methoxy-7-(3-methyl-4-{5,8,14-trioxo-15- |
| | [4-(thiophen-2-ylcarbonyl)phenyl]-2,4,9,13-tetraoxahexadecan-1- |
| | oyl}piperazin-1-yl)-4-oxo-1,4-dihydroquinoline-3-carboxylic acid |
| 69 | 1-cyclopropyl-6-fluoro-8-methoxy-4-oxo-7-(1-{5,8,14-trioxo-15-[4- |
| | (thiophen-2-ylcarbonyl)phenyl]-2,4,9,13-tetraoxahexadecan-1- |
| | oyl}octahydro-6H-pyrrolo[3,4-b]pyridin-6-yl)-1,4-dihydroquinoline- |
| | 3-carboxylic acid |
| 52 | ({[1-cyclopropyl-6-fluoro-8-methoxy-7-(3-methylpiperazin-1-yl)-4- |
| | oxo-1,4-dihydroquinolin-3-yl]carbonyl}oxy)methyl 3-({2-[4- |
| | (thiophen-2-ylcarbonyl)phenyl]propanoyl}oxy)propyl butanedioate |
| 81 | [(2R,3S,4S,5R,6S)-4-amino-6-{[(1S,2S,3R,4S,6R)-4-amino-6- |
| | {[(2S)-4-amino-2-hydroxybutanoyl]amino}-3-{[(2R,3R,4S,5S,6R)- |
| | 6-(aminomethyl)-3,4,5-trihydroxytetrahydro-2H-pyran-2-yl]oxy}-2- |
| | hydroxycyclohexyl]oxy}-3,5-dihydroxytetrahydro-2H-pyran-2- |
| | yl]methyl rel-2-(2-fluorobiphenyl-4-yl)propanoate |
| 79 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro- |
| | 2H-pyran-2-yl]oxy}-2-hydroxycyclohexyl]oxy}-3,5- |
| | dihydroxytetrahydro-2H-pyran-2-yl]methyl rel-5-(phenylcarbonyl)- |
| | 2,3-dihydro-1H-pyrrolizine-1-carboxylate |
| 19 | 7-[4-({[(2-amino-3-{[2-(2-fluorobiphenyl-4- |
| | yl)propanoyl]oxy}propanoyl)oxy]methoxy}carbonyl)-3- |
| | methylpiperazin-1-yl]-1-cyclopropyl-6-fluoro-8-methoxy-4-oxo-1,4- |
| | dihydroquinoline-3-carboxylic acid |
| 20 | 7-{4-[7-(tert-butoxycarbonyl)-15-(2-fluorobiphenyl-4-yl)-5,9,14- |
| | trioxo-2,4,13-trioxa-8-azahexadecan-1-oyl]-3-methylpiperazin-1- |
| | yl}-1-cyclopropyl-6-fluoro-8-methoxy-4-oxo-1,4-dihydroquinoline- |
| | 3-carboxylic acid |
| 21 | 7-{4-[7-carboxy-15-(2-fluorobiphenyl-4-yl)-5,9,14-trioxo-2,4,13- |
| | trioxa-8-azahexadecan-1-oyl]-3-methylpiperazin-1-yl}-1- |
| | cyclopropyl-6-fluoro-8-methoxy-4-oxo-1,4-dihydroquinoline-3- |
| | carboxylic acid |
| 29 | 7-{4-[7-amino-15-(2-fluorobiphenyl-4-yl)-5,8,14-trioxo-2,4,9,13- |
| | tetraoxahexadecan-1-oyl]-3-methylpiperazin-1-yl}-1-cyclopropyl- |
| | 6-fluoro-8-methoxy-4-oxo-1,4-dihydroquinoline-3-carboxylic acid |
| 28 | 1-cyclopropyl-6-fluoro-7-{4-[15-(2-fluorobiphenyl-4-yl)-5,8,14- |
| | trioxo-2,4,13-trioxa-9-azahexadecan-1-oyl]-3-methylpiperazin-1- |
| | yl}-8-methoxy-4-oxo-1,4-dihydroquinoline-3-carboxylic acid |
| 27 | 7-{4-[6-amino-15-(2-fluorobiphenyl-4-yl)-5,8,14-trioxo-2,4,9,13- |
| | tetraoxahexadecan-1-oyl]-3-methylpiperazin-1-yl}-1-cyclopropyl- |
| | 6-fluoro-8-methoxy-4-oxo-1,4-dihydroquinoline-3-carboxylic acid |
| 26 | 7-[4-(2-amino-3-{[2-(2-fluorobiphenyl-4-yl)propanoyl] |
| | oxy}propanoyl)-3-methylpiperazin-1-yl]-1-cyclopropyl-6-fluoro-8- |
| | methoxy-4-oxo-1,4-dihydroquinoline-3-carboxylic acid |
| 25 | 7-[4-(2-[(tert-butoxycarbonyl)amino]-3-{[2-(2-fluorobiphenyl-4- |
| | yl)propanoyl]oxy}propanoyl)-3-methylpiperazin-1-yl]-1- |
| | cyclopropyl-6-fluoro-8-methoxy-4-oxo-1,4-dihydroquinoline-3- |
| | carboxylic acid |
| 24 | 7-{4-[6-(carboxymethyl)-14-(2-fluorobiphenyl-4-yl)-5,8,13-trioxo- |
| | 2,4,12-trioxa-7-azapentadecan-1-oyl]-3-methylpiperazin-1-yl}-1- |
| | cyclopropyl-6-fluoro-8-methoxy-4-oxo-1,4-dihydroquinoline-3- |
| | carboxylic acid |
| 23 | 7-{4-[6-(2-tert-butoxy-2-oxoethyl)-14-(2-fluorobiphenyl-4-yl)- |
| | 5,8,13-trioxo-2,4,12-trioxa-7-azapentadecan-1-oyl]-3- |
| | methylpiperazin-1-yl}-1-cyclopropyl-6-fluoro-8-methoxy-4-oxo- |
| | 1,4-dihydroquinoline-3-carboxylic acid |
| 14 | 4-[({[1-cyclopropyl-6-fluoro-8-methoxy-7-(3-methylpiperazin-1- |
| | yl)-4-oxo-1,4-dihydroquinolin-3-yl]carbonyl}oxy)methyl] 1-(3-{[2- |
| | (2-fluorobiphenyl-4-yl)propanoyl]oxy}propyl) 2-aminobutanedioate |
| 13 | ({[1-(4-{[2-(2-fluorobiphenyl-4-yl)propanoyl]oxy}butanoyl) |
| | pyrrolidin-2-yl]carbonyl}oxy)methyl 1-cyclopropyl-6-fluoro-8- |
| | methoxy-7-(3-methylpiperazin-1-yl)-4-oxo-1,4-dihydroquinoline-3- |
| | carboxylate |
| 22 | 1-cyclopropyl-6-fluoro-7-(4-{[({[1-(4-{[2-(2-fluorobiphenyl-4- |
| | yl)propanoyl]oxy}butanoyl)pyrrolidin-2- |
| | yl]carbonyl}oxy)methoxy]carbonyl}-3-methylpiperazin-1-yl)-8- |
| | methoxy-4-oxo-1,4-dihydroquinoline-3-carboxylic acid |
| 50 | 1-cyclopropyl-6-fluoro-8-methoxy-7-(3-methyl-4-{5,8,14-trioxo-14- |
| | [5-(phenylcarbonyl)-2,3-dihydro-1H-pyrrolizin-1-yl]-2,4,9,13- |
| | tetraoxatetradecan-1-oyl}piperazin-1-yl)-4-oxo-1,4- |
| | dihydroquinoline-3-carboxylic acid |
| 47 | ({[1-cyclopropyl-6-fluoro-8-methoxy-7-(3-methylpiperazin-1-yl)-4- |
| | oxo-1,4-dihydroquinolin-3-yl]carbonyl}oxy)methyl 3-({[5- |
| | (phenylcarbonyl)-2,3-dihydro-1H-pyrrolizin-1- |
| | yl]carbonyl}oxy)propyl butanedioate |
| 82 | 2-amino-3-{[2-(2-fluorobiphenyl-4-yl)propanoyl]oxy}propyl 1- |
| | cyclopropyl-6-fluoro-8-methoxy-7-(3-methylpiperazin-1-yl)-4-oxo- |
| | 1,4-dihydroquinoline-3-carboxylate |
| 83 | 7-{4-[({[2-amino-3-(4-{[2-(2-fluorobiphenyl-4- |
| | yl)propanoyl]oxy}phenyl)propanoyl]oxy}methoxy)carbonyl]-3- |
| | methylpiperazin-1-yl}-1-cyclopropyl-6-fluoro-8-methoxy-4-oxo- |
| | 1,4-dihydroquinoline-3-carboxylic acid |
| 84 | 1-cyclopropyl-6-fluoro-7-{4-[(3-{[2-(2-fluorobiphenyl-4- |
| | yl)propanoyl]oxy}propoxy)carbonyl]-3-methylpiperazin-1-yl}-8- |
| | methoxy-4-oxo-1,4-dihydroquinoline-3-carboxylic acid |
| 85 | 7-[4-({[(3-carboxy-3-{[2-(2-fluorobiphenyl-4- |
| | yl)propanoyl]amino}propanoyl)oxy]methoxy}carbonyl)-3- |
| | methylpiperazin-1-yl]-1-cyclopropyl-6-fluoro-8-methoxy-4-oxo-1,4- |
| | dihydroquinoline-3-carboxylic acid |
| 86 | 7-{4-[7-amino-16-(2-fluorobiphenyl-4-yl)-5,9,15-trioxo-2,4,10,14- |
| | tetraoxaheptadecan-1-oyl]-3-methylpiperazin-1-yl}-1-cyclopropyl- |
| | 6-fluoro-8-methoxy-4-oxo-1,4-dihydroquinoline-3-carboxylic acid |
| 87 | 7-{4-[7-amino-15-(2-fluorobiphenyl-4-yl)-5,8,14-trioxo-2,4,9,13- |
| | tetraoxahexadecan-1-oyl]-3-methylpiperazin-1-yl}-1-cyclopropyl- |
| | 6-fluoro-8-methoxy-4-oxo-1,4-dihydroquinoline-3-carboxylic acid |
| 88 | 1-cyclopropyl-6-fluoro-7-{4-[15-(2-fluorobiphenyl-4-yl)-5,8,14- |
| | trioxo-2,4,13-trioxa-9-azahexadecan-1-oyl]-3-methylpiperazin-1- |
| | yl}-8-methoxy-4-oxo-1,4-dihydroquinoline-3-carboxylic acid |
| 89 | 7-{4-[6-amino-15-(2-fluorobiphenyl-4-yl)-5,8,14-trioxo-2,4,9,13- |
| | tetraoxahexadecan-1-oyl]-3-methylpiperazin-1-yl}-1-cyclopropyl- |
| | 6-fluoro-8-methoxy-4-oxo-1,4-dihydroquinoline-3-carboxylic acid |
| 90 | 7-[4-(2-amino-3-{[2-(2-fluorobiphenyl-4- |
| | yl)propanoyl]oxy}propanoyl)-3-methylpiperazin-1-yl]-1- |
| | cyclopropyl-6-fluoro-8-methoxy-4-oxo-1,4-dihydroquinoline-3- |
| | carboxylic acid |
| 91 | 7-[4-(2-[(tert-butoxycarbonyl)amino]-3-{[2-(2-fluorobiphenyl-4- |
| | yl)propanoyl]oxy}propanoyl)-3-methylpiperazin-1-yl]-1- |
| | cyclopropyl-6-fluoro-8-methoxy-4-oxo-1,4-dihydroquinoline-3- |
| | carboxylic acid |
| 92 | 7-{4-[6-(carboxymethyl)-14-(2-fluorobiphenyl-4-yl)-5,8,13-trioxo- |
| | 2,4,12-trioxa-7-azapentadecan-1-oyl]-3-methylpiperazin-1-yl}-1- |
| | cyclopropyl-6-fluoro-8-methoxy-4-oxo-1,4-dihydroquinoline-3- |
| | carboxylic acid |
| 93 | 7-{4-[6-(2-tert-butoxy-2-oxoethyl)-14-(2-fluorobiphenyl-4-yl)- |
| | 5,8,13-trioxo-2,4,12-trioxa-7-azapentadecan-1-oyl]-3- |
| | methylpiperazin-1-yl}-1-cyclopropyl-6-fluoro-8-methoxy-4-oxo- |
| | 1,4-dihydroquinoline-3-carboxylic acid |
| 94 | 4-[({[1-cyclopropyl-6-fluoro-8-methoxy-7-(3-methylpiperazin-1-yl)- |
| | 4-oxo-1,4-dihydroquinolin-3-yl]carbonyl}oxy)methyl] 1-(3-{[2-(2- |
| | fluorobiphenyl-4-yl)propanoyl]oxy}propyl)2-aminobutanedioate |
| 95 | ({[1-(4-{[2-(2-fluorobiphenyl-4-yl)propanoyl]oxy}butanoyl)pyrrolidin- |
| | 2-yl]carbonyl}oxy)methyl 1-cyclopropyl-6-fluoro-8-methoxy-7-(3- |
| | methylpiperazin-1-yl)-4-oxo-1,4-dihydroquinoline-3-carboxylate |
| 96 | 1-cyclopropyl-6-fluoro-7-(4-{[({[1-(4-{[2-(2-fluorobiphenyl-4- |
| | yl)propanoyl]oxy}butanoyl)pyrrolidin-2- |
| | yl]carbonyl}oxy)methoxy]carbonyl}-3-methylpiperazin-1-yl)-8- |
| | methoxy-4-oxo-1,4-dihydroquinoline-3-carboxylic acid |
| 97 | 1-cyclopropyl-6-fluoro-8-methoxy-7-(3-methyl-4-{5,8,14-trioxo-14- |
| | [5-(phenylcarbonyl)-2,3-dihydro-1H-pyrrolizin-1-yl]-2,4,9,13- |
| | tetraoxatetradecan-1-oyl}piperazin-1-yl)-4-oxo-1,4- |
| | dihydroquinoline-3-carboxylic acid |
| 98 | ({[1-cyclopropyl-6-fluoro-8-methoxy-7-(3-methylpiperazin-1-yl)-4- |
| | oxo-1,4-dihydroquinolin-3-yl]carbonyl}oxy)methyl 3-({[5- |
| | (phenylcarbonyl)-2,3-dihydro-1H-pyrrolizin-1- |
| | yl]carbonyl}oxy)propyl butanedioate |
| 99 | ({[1-cyclopropyl-6-fluoro-8-methoxy-7-(3-methylpiperazin-1-yl)-4- |
| | oxo-1,4-dihydroquinolin-3-yl]carbonyl}oxy)methyl {[2-(2- |
| | fluorobiphenyl-4-yl)propanoyl]oxy}methyl butanedioate |
| 100 | 2-[({7-[4-(tert-butoxycarbonyl)-3-methylpiperazin-1-yl]-1- |
| | cyclopropyl-6-fluoro-8-methoxy-4-oxo-1,4-dihydroquinolin-3- |
| | yl}carbonyl)amino]ethyl 5-(phenylcarbonyl)-2,3-dihydro-1H- |
| | pyrrolizine-1-carboxylate |
| 101 | 2-(2-{2-[({1-[(4-chlorophenyl)carbonyl]-5-methoxy-2-methyl-1H- |
| | indol-3-yl}acetyl)oxy]ethoxy}ethoxy)ethyl 1-cyclopropyl-6-fluoro-8- |
| | methoxy-7-(octahydro-6H-pyrrolo[3,4-b]pyridin-6-yl)-4-oxo-1,4- |
| | dihydroquinoline-3-carboxylate |
| 102 | 2-(2-{2-[({1-[(4-chlorophenyl)carbonyl]-5-methoxy-2-methyl-1H- |
| | indol-3-yl}acetyl)oxy]ethoxy}ethoxy)ethyl 7-[1-(tert- |
| | butoxycarbonyl)octahydro-6H-pyrrolo[3,4-b]pyridin-6-yl]-1- |
| | cyclopropyl-6-fluoro-8-methoxy-4-oxo-1,4-dihydroquinoline-3- |
| | carboxylate |
| 103 | 2-{2-[2-({[5-(phenylcarbonyl)-2,3-dihydro-1H-pyrrolizin-1- |
| | yl]carbonyl}oxy)ethoxy]ethoxy}ethyl 1-cyclopropyl-6-fluoro-8- |
| | methoxy-7-(octahydro-6H-pyrrolo[3,4-b]pyridin-6-yl)-4-oxo-1,4- |
| | dihydroquinoline-3-carboxylate |
| 104 | 2-{2-[2-({[5-(phenylcarbonyl)-2,3-dihydro-1H-pyrrolizin-1- |
| | yl]carbonyl}oxy)ethoxy]ethoxy}ethyl 7-[1-(tert- |
| | butoxycarbonyl)octahydro-6H-pyrrolo[3,4-b]pyridin-6-yl]-1- |
| | cyclopropyl-6-fluoro-8-methoxy-4-oxo-1,4-dihydroquinoline-3- |
| | carboxylate |
| 105 | 2-(2-{2-[({1-[(4-chlorophenyl)carbonyl]-5-methoxy-2-methyl-1H- |
| | indol-3-yl}acetyl)oxy]ethoxy}ethoxy)ethyl 1-cyclopropyl-6-fluoro-8- |
| | methoxy-7-(3-methylpiperazin-1-yl)-4-oxo-1,4-dihydroquinoline-3- |
| | carboxylate |
| 106 | 2-{2-[2-({[5-(phenylcarbonyl)-2,3-dihydro-1H-pyrrolizin-1- |
| | yl]carbonyl}oxy)ethoxy]ethoxy}ethyl 7-[4-(tert-butoxycarbonyl)-3- |
| | methylpiperazin-1-yl]-1-cyclopropyl-6-fluoro-8-methoxy-4-oxo-1,4- |
| | dihydroquinoline-3-carboxylate |
| 107 | 2-{2-[2-({[5-(phenylcarbonyl)-2,3-dihydro-1H-pyrrolizin-1- |
| | yl]carbonyl}oxy)ethoxy]ethoxy}ethyl 1-cyclopropyl-6-fluoro-8- |
| | methoxy-7-(3-methylpiperazin-1-yl)-4-oxo-1,4-dihydroquinoline-3- |
| | carboxylate |
| 108 | 2-(2-{2-[({1-[(4-chlorophenyl)carbonyl]-5-methoxy-2-methyl-1H- |
| | indol-3-yl}acetyl)oxy]ethoxy}ethoxy)ethyl 7-[4-(tert- |
| | butoxycarbonyl)-3-methylpiperazin-1-yl]-1-cyclopropyl-6-fluoro-8- |
| | methoxy-4-oxo-1,4-dihydroquinoline-3-carboxylate |
| 109 | 7-[4-({1-[(4-chlorophenyl)carbonyl]-5-methoxy-2-methyl-1H-indol- |
| | 3-yl}acetyl)-3-methylpiperazin-1-yl]-1-cyclopropyl-6-fluoro-8- |
| | methoxy-4-oxo-1,4-dihydroquinoline-3-carboxylic acid |

In another embodiment the compounds disclosed herein comprise at least one non-steroidal anti-inflammatory drug and at least one antibiotic drug.

In another embodiment the compounds disclosed herein comprise at least one non-steroidal anti-inflammatory drug and one antibiotic drug and at least one linker selected from Table 1.

In another embodiment the compounds disclosed herein comprise at least one non-steroidal anti-inflammatory drug and one antibiotic drug and least one linker selected from Table 1 and one pro-drug moiety selected from Table 2.

In another embodiment the compounds disclosed herein comprise one gatifloxacin moiety and one flubiprofen moiety, such as:
2-[2-(2-{[2-(2-fluorobiphenyl-4-yl)propanoyl]oxy}ethoxy)ethoxy]ethyl 1-cyclopropyl-6-fluoro-8-methoxy-7-(3-methylpiperazin-1-yl)-4-oxo-1,4-dihydroquinoline-3-carboxylate 3-{[2-(2-fluorobiphenyl-4-yl)propanoyl]oxy}propyl 1-cyclopropyl-6-fluoro-8-methoxy-7-(3-methylpiperazin-1-yl)-4-oxo-1,4-dihydroquinoline-3-carboxylate;
2-{[2-(2-fluorobiphenyl-4-yl)propanoyl]oxy}ethyl 1-cyclopropyl-6-fluoro-8-methoxy-7-(3-methylpiperazin-1-yl)-4-oxo-1,4-dihydroquinoline-3-carboxylate;
3-{[2-(2-fluorobiphenyl-4-yl)propanoyl]oxy}propyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
2-{[2-(2-fluorobiphenyl-4-yl)propanoyl]oxy}ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
2-[2-(2-{[2-(2-fluorobiphenyl-4-yl)propanoyl]oxy}ethoxy)ethoxy]ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
({[1-cyclopropyl-6-fluoro-8-methoxy-7-(3-methylpiperazin-1-yl)-4-oxo-1,4-dihydroquinolin-3-yl]carbonyl}oxy)methyl 3-{[2-(2-fluorobiphenyl-4-yl)propanoyl]oxy}propyl butanedioate;
{[(3-{[2-(2-fluorobiphenyl-4-yl)propanoyl]oxy}propoxy)carbonyl]oxy}methyl 1-cyclopropyl-6-fluoro-8-methoxy-7-(3-methylpiperazin-1-yl)-4-oxo-1,4-dihydroquinoline-3-carboxylate;
[5-({[2-(2-fluorobiphenyl-4-yl)propanoyl]oxy}methyl)-2-oxo-1,3-dioxol-4-yl]methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-(4-{[5-({[2-(2-fluorobiphenyl-4-yl)propanoyl]oxy}methyl)-2-oxo-1,3-dioxol-4-yl]methyl}-3-methylpiperazin-1-yl)-8-methoxy-4-oxo-1,4-dihydroquinoline-3-carboxylic acid;
{[2-(2-fluorobiphenyl-4-yl)propanoyl]oxy}methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-{4-[({[2-(2-fluorobiphenyl-4-yl)propanoyl]oxy}methoxy)carbonyl]-3-methylpiperazin-1-yl}-8-methoxy-4-oxo-1,4-dihydroquinoline-3-carboxylic acid;
[(4-{[2-(2-fluorobiphenyl-4-yl)propanoyl]oxy}butanoyl)oxy]methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-[4-({[(4-{[2-(2-fluorobiphenyl-4-yl)propanoyl]oxy}butanoyl)oxy]methoxy}carbonyl)-3-methylpiperazin-1-yl]-8-methoxy-4-oxo-1,4-dihydroquinoline-3-carboxylic acid;
7-[4-({[(2-amino-3-{[2-(2-fluorobiphenyl-4-yl)propanoyl]oxy}propanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
7-{4-[7-(tert-butoxycarbonyl)-15-(2-fluorobiphenyl-4-yl)-5,9,14-trioxo-2,4,13-trioxa-8-azahexadecan-1-oyl]-3-methylpiperazin-1-yl}-1-cyclopropyl-6-fluoro-8-methoxy-4-oxo-1,4-dihydroquinoline-3-carboxylic acid;
7-{4-[7-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
1-cyclopropyl-6-fluoro-7-{4-[15-(2-fluorobiphenyl-4-yl)-5,8,14-trioxo-2,4,13-trioxa-9-azahexadecan-1-oyl]-3-methylpiperazin-1-yl}-8-methoxy-4-oxo-1,4-dihydroquinoline-3-carboxylic acid;
7-{4-[6-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid; 7-[4-(2-amino-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-[4-(2-[(tert-butoxycarbonyl)amino]-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-{4-[6-(carboxymethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
7-{4-[6-(2-tert-butoxy-2-oxoethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
4-[({[1-cyclopropyl-6-fluoro-8-methoxy-7-(3-methylpiperazin-1-yl)-4-oxo-1,4-dihydroquinolin-3-yl]carbonyl}oxy)methyl]1-(3-{[2-(2-fluorobiphenyl-4-yl)propanoyl]oxy}propyl) 2-aminobutanedioate;
({[1-(4-{[2-(2-fluorobiphenyl-4-yl)propanoyl]oxy}butanoyl)pyrrolidin-2-yl]carbonyl}oxy)methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-(4-{[({[1-(4-{[2-(2-fluorobiphenyl-4-yl)propanoyl]oxy}butanoyl)pyrrolidin-2-yl]carbonyl}oxy)methoxy]carbonyl}-3-methylpiperazin-1-yl)-8-methoxy-4-oxo-1,4-dihydroquinoline-3-carboxylic acid;
2-amino-3-{[2-(2-fluorobiphenyl-4-yl)propanoyl]oxy}propyl 1-cyclopropyl-6-fluoro-8-methoxy-7-(3-methylpiperazin-1-yl)-4-oxo-1,4-dihydroquinoline-3-carboxylate;
7-{4-[({[2-amino-3-(4-{[2-(2-fluorobiphenyl-4-yl)propanoyl]oxy}phenyl)propanoyl]oxy}methoxy)carbonyl]-3-methyl piperazin-1-yl}-1-cyclopropyl-6-fluoro-8-methoxy-4-oxo-1,4-dihydroquinoline-3-carboxylic acid;
1-cyclopropyl-6-fluoro-7-{4-[(3-{[2-(2-fluorobiphenyl-4-yl)propanoyl]oxy}propoxy)carbonyl]-3-methylpiperazin-1-yl}-8-methoxy-4-oxo-1,4-dihydroquinoline-3-carboxylic acid;
7-[4-({[(3-carboxy-3-{[2-(2-fluorobiphenyl-4-yl)propanoyl]amino}propanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
7-{4-[7-amino-16-(2-fluorobiphenyl-4-yl)-5,9,15-trioxo-2,4,10,14-tetraoxaheptadecan-1-oyl]-3-methylpiperazin-1-yl}-1-cyclopropyl-6-fluoro-8-methoxy-4-oxo-1,4-dihydroquinoline-3-carboxylic acid;
7-{4-[7-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
1-cyclopropyl-6-fluoro-7-{4-[15-(2-fluorobiphenyl-4-yl)-5,8,14-trioxo-2,4,13-trioxa-9-azahexadecan-1-oyl]-3-methylpiperazin-1-yl}-8-methoxy-4-oxo-1,4-dihydroquinoline-3-carboxylic acid;
7-{4-[6-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
({[1-cyclopropyl-6-fluoro-8-methoxy-7-(3-methyl piperazin-1-yl)-4-oxo-1,4-dihydroquinolin-3-yl]carbonyl}oxy)methyl{[2-(2-fluorobiphenyl-4-yl)propanoyl]oxy}methyl butanedioate;
7-{4-[6-(carboxymethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
7-{4-[6-(2-tert-butoxy-2-oxoethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
4-[({[1-cyclopropyl-6-fluoro-8-methoxy-7-(3-methylpiperazin-1-yl)-4-oxo-1,4-dihydroquinolin-3-yl]carbonyl}oxy)methyl]1-(3-{[2-(2-fluorobiphenyl-4-yl)propanoyl]oxy}propyl) 2-aminobutanedioate;
({[1-(4-{[2-(2-fluorobiphenyl-4-yl)propanoyl]oxy}butanoyl)pyrrolidin-2-yl]carbonyl}oxy)methyl 1-cyclopropyl-6-fluoro-8-methoxy;-7-(3-methylpiperazin-1-yl)-4-oxo-1,4-dihydroquinoline-3-carboxylate;
1-cyclopropyl-6-fluoro-7-(4-{[({[1-(4-{[2-(2-fluorobiphenyl-4-yl)propanoyl]oxy}butanoyl)pyrrolidin-2-yl]carbonyl}oxy)methoxy]carbonyl}-3-methylpiperazin-1-yl)-8-methoxy-4-oxo-1,4-dihydroquinoline-3-carboxylic acid;
7-[4-(2-amino-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-[4-(2-[(tert-butoxycarbonyl)amino]-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid.

In another embodiment the compounds disclosed herein comprise one gatifloxacin moiety and one indomethacin moiety, such as:
2-(2-{2-[({1-[(4-chlorophenyl)carbonyl]-5-methoxy-2-methyl-1H-indol-3-yl}acetyl)oxy]ethoxy}ethoxy)ethyl 7-[4-(tert-butoxycarbonyl)-3-methylpiperazin-1-yl]-1-cyclopropyl-6-fluoro-8-methoxy-4-oxo-1,4-dihydroquinoline-3-carboxylate;
7-[4-({1-[(4-chlorophenyl)carbonyl]-5-methoxy-2-methyl-1H-indol-3-yl}acetyl)-3-methylpiperazin-1-yl]-1-cyclopropyl-6-fluoro-8-methoxy-4-oxo-1,4-dihydroquinoline-3-carboxylic acid;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(3-methylpiperazin-1-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(3-methylpiperazin-1-yl)-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl 1-cyclopropyl-6-fluoro-8-methoxy-7-(3-methylpiperazin-1-yl)-4-oxo-1,4-dihydroquinoline-3-carboxylate;
7-[4-({[({1-[(4-chlorophenyl)carbonyl]-5-methoxy-2-methyl-1H-indol-3-yl}acetyl)oxy]methoxy}carbonyl)-3-methylpiperazin-1-yl]-1-cyclopropyl-6-fluoro-8-methoxy-4-oxo-1,4-dihydroquinoline-3-carboxylic acid;
[({1-[(4-chlorophenyl)carbonyl]-5-methoxy-2-methyl-1H-indol-3-yl}acetyl)oxy]methyl 1-cyclopropyl-6-fluoro-8-methoxy-7-(3-methylpiperazin-1-yl)-4-oxo-1,4-dihydroquinoline-3-carboxylate;
2-[({1-[(4-chlorophenyl)carbonyl]-5-methoxy-2-methyl-1H-indol-3-yl}acetyl)oxy]ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
[({3-[({1-[(4-chlorophenyl)carbonyl]-5-methoxy-2-methyl-1H-indol-3-yl}acetyl)oxy]propoxy}carbonyl)oxy]methyl 1-cyclopropyl-6-fluoro-8-methoxy-7-(3-methylpiperazin-1-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl({[1-cyclopropyl-6-fluoro-8-methoxy-7-(3-methylpiperazin-1-yl)-4-oxo-1,4-dihydroquinolin-3-yl]carbonyl}oxy)methyl butanedioate;
7-(4-{[({[({1-[(4-chlorophenyl)carbonyl]-5-methoxy-2-methyl-1H-indol-3-yl}acetyl)oxy]acetyl}oxy)methoxy]carbonyl}-3-methyl piperazin-1-yl)-1-cyclopropyl-6-fluoro-8-methoxy-4-oxo-1,4-dihydroquinoline-3-carboxylic acid;
7-(4-{[({4-[({1-[(4-chlorophenyl)carbonyl]-5-methoxy-2-methyl-1H-inden-3-yl}acetyl)oxy]butanoyl}oxy)methoxy]carbonyl}-3-methylpiperazin-1-yl)-1-cyclopropyl-6-fluoro-8-methoxy-4-oxo-1,4-dihydroquinoline-3-carboxylic acid.

In another embodiment the compounds disclosed herein comprise one gatifloxacin moiety and one diclofenac moiety, such as:
1-cyclopropyl-7-[4-({[({2-[(2,6-dichlorophenyl)amino]phenyl}acetyl)oxy]methoxy}carbonyl)-3-methylpiperazin-1-yl]-6-fluoro-8-methoxy-4-oxo-1,4-dihydroquinoline-3-carboxylic acid;
[({2-[(2,6-dichlorophenyl)amino]phenyl}acetyl)oxy]methyl 1-cyclopropyl-6-fluoro-8-methoxy-7-(3-methylpiperazin-1-yl)-4-oxo-1,4-dihydroquinoline-3-carboxylate;
3-[({2-[(2,6-dichlorophenyl)amino]phenyl}acetyl)oxy]propyl 1-cyclopropyl-6-fluoro-8-methoxy-7-(3-methylpiperazin-1-yl)-4-oxo-1,4-dihydroquinoline-3-carboxylate.

In another embodiment the compounds disclosed herein comprise one gatifloxacin moiety and one ketorolac moiety, such as:
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(3-methylpiperazin-1-yl)-4-oxo-1,4-dihydroquinoline-3-carboxylate;
2-({[1-cyclopropyl-6-fluoro-8-methoxy-7-(3-methylpiperazin-1-yl)-4-oxo-1,4-dihydroquinolin-3-yl]carbonyl}amino)ethyl 5-(phenylcarbonyl)-2,3-dihydro-1H-pyrrolizine-1-carboxylate;
({[5-(phenylcarbonyl)-2,3-dihydro-1H-pyrrolizin-1-yl]carbonyl}oxy)methyl 1-cyclopropyl-6-fluoro-8-methoxy-7-(3-methylpiperazin-1-yl)-4-oxo-1,4-dihydroquinoline-3-carboxylate;
({[1-cyclopropyl-6-fluoro-8-methoxy-7-(3-methylpiperazin-1-yl)-4-oxo-1,4-dihydroquinolin-3-yl]carbonyl}oxy)methyl 3-({[5-(phenylcarbonyl)-2,3-dihydro-1H-pyrrolizin-1-yl]carbonyl}oxy)propyl butanedioate;
1-cyclopropyl-6-fluoro-8-methoxy-7-(3-methyl-4-{[({[5-(phenylcarbonyl)-2,3-dihydro-1H-pyrrolizin-1-yl]carbonyl}oxy)methoxy]carbonyl}piperazin-1-yl)-4-oxo-1,4-dihydroquinoline-3-carboxylic acid;
1-cyclopropyl-6-fluoro-8-methoxy-7-{3-methyl-4-[({[4-({[5-(phenylcarbonyl)-2,3-dihydro-1H-pyrrolizin-1-yl]carbonyl}oxy)butanoyl]oxy}methoxy)carbonyl]piperazin-1-yl}-4-oxo-1,4-dihydroquinoline-3-carboxylic acid;
1-cyclopropyl-6-fluoro-8-methoxy-7-(3-methyl-4-{5,8,14-trioxo-14-[5-(phenylcarbonyl)-2,3-dihydro-1H-pyrrolizin-1-yl]-2,4,9,13-tetraoxatetradecan-1-oyl}piperazin-1-yl)-4-oxo-1,4-dihydroquinoline-3-carboxylic acid;
1-cyclopropyl-6-fluoro-8-methoxy-7-(3-methyl-4-{5,8,14-trioxo-14-[5-(phenylcarbonyl)-2,3-dihydro-1H-pyrrolizin-1-yl]-2,4,9,13-tetraoxatetradecan-1-oyl}piperazin-1-yl)-4-oxo-1,4-dihydroquinoline-3-carboxylic acid;
({[1-cyclopropyl-6-fluoro-8-methoxy-7-(3-methyl piperazin-1-yl)-4-oxo-1,4-dihydroquinolin-3-yl]carbonyl}oxy)methyl 3-({[5-(phenylcarbonyl)-2,3-dihydro-1H-pyrrolizin-1-yl]carbonyl}oxy)propyl butanedioate;
2-[({7-[4-(tert-butoxycarbonyl)-3-methylpiperazin-1-yl]-1-cyclopropyl-6-fluoro-8-methoxy-4-oxo-1,4-dihydroquinolin-3-yl}carbonyl)amino]ethyl 5-(phenylcarbonyl)-2,3-dihydro-1H-pyrrolizine-1-carboxylate;
2-{2-[2-({[5-(phenylcarbonyl)-2,3-dihydro-1H-pyrrolizin-1-yl]carbonyl}oxy)ethoxy]ethoxy}ethyl 7-[4-(tert-butoxycarbonyl)-3-methylpiperazin-1-yl]-1-cyclopropyl-6-fluoro-8-methoxy-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(3-methylpiperazin-1-yl)-4-oxo-1,4-dihydroquinoline-3-carboxylate.

In another embodiment the compounds disclosed herein comprise one gatifloxacin moiety and one suprofen moiety, such as:
1-cyclopropyl-6-fluoro-8-methoxy-7-(3-methyl-4-{[({2-[4-(thiophen-2-ylcarbonyl)phenyl]propanoyl}oxy)methoxy]carbonyl}piperazin-1-yl)-4-oxo-1,4-dihydroquinoline-3-carboxylic acid;
({2-[4-(thiophen-2-ylcarbonyl)phenyl]propanoyl}oxy)methyl 1-cyclopropyl-6-fluoro-8-methoxy-7-(3-methylpiperazin-1-yl)-4-oxo-1,4-dihydroquinoline-3-carboxylate;
({[1-cyclopropyl-6-fluoro-8-methoxy-7-(3-methylpiperazin-1-yl)-4-oxo-1,4-dihydroquinolin-3-yl]carbonyl}oxy)methyl 3-({2-[4-(thiophen-2-ylcarbonyl)phenyl]propanoyl}oxy)propyl butanedioate;
1-cyclopropyl-6-fluoro-8-methoxy-7-(3-methyl-4-{5,8,14-trioxo-15-[4-(thiophen-2-ylcarbonyl)phenyl]-2,4,9,13-tetraoxahexadecan-1-oyl}piperazin-1-yl)-4-oxo-1,4-dihydroquinoline-3-carboxylic acid.

In another embodiment the compounds disclosed herein comprise one moxifloxacin moiety and one flubiprofen moiety, such as:
2-{[2-(2-fluorobiphenyl-4-yl)propanoyl]oxy}ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
1-cyclopropyl-6-fluoro-7-{1-[({[2-(2-fluorobiphenyl-4-yl)propanoyl]oxy}methoxy)carbonyl]octahydro-6H-pyrrolo[3,4-b]pyridin-6-yl}-8-methoxy-4-oxo-1,4-dihydroquinoline-3-carboxylic acid;
1-cyclopropyl-6-fluoro-7-[1-({[(4-{[2-(2-fluorobiphenyl-4-yl)propanoyl]oxy}butanoyl)oxy]methoxy}carbonyl)octahydro-6H-pyrrolo[3,4-b]pyridin-6-yl]-8-methoxy-4-oxo-1,4-dihydroquinoline-3-carboxylic acid;
1-cyclopropyl-6-fluoro-7-{1-[15-(2-fluorobiphenyl-4-yl)-5,8,14-trioxo-2,4,9,13-tetraoxahexadecan-1-oyl]octahydro-6H-pyrrolo[3,4-b]pyridin-6-yl}-8-methoxy-4-oxo-1,4-dihydroquinoline-3-carboxylic acid.

In another embodiment the compounds disclosed herein comprise one moxifloxacin moiety and one indometacin moiety, such as:
2-(2-{2-[({1-[(4-chlorophenyl)carbonyl]-5-methoxy-2-methyl-1H-indol-3-yl}acetyl)oxy]ethoxy}ethoxy)ethyl rel-1-cyclopropyl-7-[(4aR,7aR)-4a,7a-dimethyloctahydro-6H-pyrrolo[3,4-b]pyridin-6-yl]-6-fluoro-8-methoxy-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate;
[({1-[(4-chlorophenyl)carbonyl]-5-methoxy-2-methyl-1H-indol-3-yl}acetyl)oxy]methyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate;
7-[1-({[({1-[(4-chlorophenyl)carbonyl]-5-methoxy-2-methyl-1H-inden-3-yl}acetyl)oxy]methoxy}carbonyl)octahydro-6H-pyrrolo[3,4-b]pyridin-6-yl]-1-cyclopropyl-6-fluoro-8-methoxy-4-oxo-1,4-dihydroquinoline-3-carboxylic acid;
7-(1-{[({4-[({1-[(4-chlorophenyl)carbonyl]-5-methoxy-2-methyl-1H-inden-3-yl}acetyl)oxy]butanoyl}oxy)methoxy]carbonyl}octahydro-6H-pyrrolo[3,4-b]pyridin-6-yl)-1-cyclopropyl-6-fluoro-8-methoxy-4-oxo-1,4-dihydroquinoline-3-carboxylic acid;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 7-[1-(tert-butoxycarbonyl)octahydro-6H-pyrrolo[3,4-b]pyridin-6-yl]-1-cyclopropyl-6-fluoro-8-methoxy-4-oxo-1,4-dihydroquinoline-3-carboxylate.

In another embodiment the compounds disclosed herein comprise one moxifloxacin moiety and one diclofenac moiety, such as:
3-[({2-[(2,6-dichlorophenyl)amino]phenyl}acetyl)oxy]propyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate.

In another embodiment the compounds disclosed herein comprise one moxifloxacin moiety and one ketorolac moiety, such as:
2-{2-[2-({[5-(phenylcarbonyl)-2,3-dihydro-1H-pyrrolizin-1-yl]carbonyl}oxy)ethoxy]ethoxy}ethyl rel-1-cyclopropyl-7-[(4aR,7aR)-4a,7a-dimethyloctahydro-6H-pyrrolo[3,4-b]pyridin-6-yl]-6-fluoro-8-methoxy-4-oxo-1,4-dihydroquinoline-3-carboxylate;
2-[({1-cyclopropyl-7-[(4aR,7aR)-4a,7a-dimethyloctahydro-6H-pyrrolo[3,4-b]pyridin-6-yl]-6-fluoro-8-methoxy-4-oxo-1,4-dihydroquinolin-3-yl}carbonyl)amino]ethyl rel-5-(phenylcarbonyl)-2,3-dihydro-1H-pyrrolizine-1-carboxylate;

rel-1-cyclopropyl-6-fluoro-8-methoxy-4-oxo-7-[(4aR,7aR)-1-{[({[5-(phenylcarbonyl)-2,3-dihydro-1H-pyrrolizin-1-yl]carbonyl}oxy)methoxy]carbonyl}octahydro-6H-pyrrolo[3,4-b]pyridin-6-yl]-1,4-dihydroquinoline-3-carboxylic acid; 1-cyclopropyl-6-fluoro-8-methoxy-4-oxo-7-{1-[({[4-({[5-(phenylcarbonyl)-2,3-dihydro-1H-pyrrolizin-1-yl]carbonyl}oxy)butanoyl]oxy}methoxy)carbonyl]octahydro-6H-pyrrolo[3,4-b]pyridin-6-yl}-1,4-dihydroquinoline-3-carboxylic acid;
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl 7-[1-(tert-butoxycarbonyl)octahydro-6H-pyrrolo[3,4-b]pyridin-6-yl]-1-cyclopropyl-6-fluoro-8-methoxy-4-oxo-1,4-dihydroquinoline-3-carboxylate.

In another embodiment the compounds disclosed herein comprise one moxifloxacin moiety and one suprofen moiety, such as:
1-cyclopropyl-6-fluoro-8-methoxy-4-oxo-7-(1-{5,8,14-trioxo-15-[4-(thiophen-2-ylcarbonyl)phenyl]-2,4,9,13-tetraoxahexadecan-1-oyl}octahydro-6H-pyrrolo[3,4-b]pyridin-6-yl)-1,4-dihydroquinoline-3-carboxylic acid.

In another embodiment the compounds disclosed herein comprise one chloramfenicol moiety and one indometacin moiety, such as:
1-cyclopropyl-6-fluoro-8-methoxy-4-oxo-7-(1-{5,8,14-trioxo-15-[4-(thiophen-2-ylcarbonyl)phenyl]-2,4,9,13-tetraoxahexadecan-1-oyl}octahydro-6H-pyrrolo[3,4-b]pyridin-6-yl)-1,4-dihydroquinoline-3-carboxylic acid;
2-[(dichloroacetyl)amino]-3-hydroxy-3-(4-nitrophenyl)propyl 4-[({1-[(4-chlorophenyl)carbonyl]-5-methoxy-2-methyl-1H-indol-3-yl}acetyl)oxy]butanoate; 2-[(dichloroacetyl)amino]-3-hydroxy-3-(4-nitrophenyl)propyl {1-[(4-chlorophenyl)carbonyl]-5-methoxy-2-methyl-1H-indol-3-yl}acetate;
2-{2-[(dichloroacetyl)amino]-3-hydroxy-3-(4-nitrophenyl)propoxy}-2-oxoethyl {1-[(4-chlorophenyl)carbonyl]-5-methoxy-2-methyl-1H-indol-3-yl}acetate.

In another embodiment the compounds disclosed herein comprise one chloramfenicol moiety and one flubiprofen moiety, such as:
2-[(dichloroacetyl)amino]-3-hydroxy-1-(4-nitrophenyl)propyl 4-{[2-(2-fluorobiphenyl-4-yl)propanoyl]oxy}butanoate.

In another embodiment the compounds disclosed herein comprise one tobramycin moiety and one indomethacin moiety, such as:
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-4-[({1-[(4-chlorophenyl)carbonyl]-5-methoxy-2-methyl-1H-indol-3-yl}acetyl)oxy]butanoate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-{1-[(4-chlorophenyl)carbonyl]-5-methoxy-2-methyl-1H-indol-3-yl}acetate.

In another embodiment the compounds disclosed herein comprise one tobramycin moiety and one ketorolac moiety, such as:
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-5-(phenylcarbonyl)-2,3-dihydro-1H-pyrrolizine-1-carboxylate.

In another embodiment the compounds disclosed herein comprise one tobramycin moiety and one flubiprofen moiety, such as:
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-4-{[2-(2-fluorobiphenyl-4-yl)propanoyl]oxy}butanoate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-2-(2-fluorobiphenyl-4-yl)propanoate.

In another embodiment the compounds disclosed herein comprise one amikacin moiety and one flubiprofen moiety, such as:
[(2R,3S,4S,5R,6S)-4-amino-6-{[(1S,2S,3R,4S,6R)-4-amino-6-{[(2S)-4-amino-2-hydroxybutanoyl]amino}-3-{[(2R,3R,4S,5S,6R)-6-(aminomethyl)-3,4,5-trihydroxytetrahydro-2H-pyran-2-yl]oxy}-2-hydroxycyclohexyl]oxy}-3,5-dihydroxytetrahydro-2H-pyran-2-yl]methyl rel-2-(2-fluorobiphenyl-4-yl)propanoate.

In another embodiment the invention provides a compound comprising an antibiotic moiety and a nonsteroidal anti-inflammatory drug moiety, which are connected via two separate covalent bonds to a linker such that said compound degrades in vivo to yield the antibiotic and the nonsteroidal anti-inflammatory drug, wherein each bond is an ester bond or an amide bond.

In another embodiment the invention provides a hybrid compound wherein the nonsteroidal anti-inflammatory drug moiety is selected from the group consisting of indomethacin, diclofenac, flurbiprofen, ketorolac and suprofen.

In another embodiment the invention provides a hybrid compound wherein the antibiotic drug moiety is selected from the group consisting of: gatifloxacin, moxifloxacin, chloramphenicol, tobramycin and amikacin.

In another aspect the invention provides compounds which may comprise a linker moiety selected from, but not limited to, an ester, a carboxylate, a carbonyl, a carbonate, an amido, a carbamate, a ketone, an amino, an oxo, an ethylene glycol, a polyethylene glycol, an ethylene.

In another embodiment the invention provides a hybrid compound, comprising a gatifloxacin moiety and a nonsteroidal anti-inflammatory drug moiety selected from the group consisting of: indomethacin, diclofenac, flurbiprofen and suprofen.

In another embodiment the invention provides a hybrid compound, comprising a moxifloxacin moiety and a nonsteroidal anti-inflammatory drug moiety selected from the group consisting of: indomethacin, diclofenac, and suprofen.

In another embodiment the invention provides a hybrid compound, comprising a chloramphenicol moiety and a nonsteroidal anti-inflammatory drug moiety selected from the group consisting of: indomethacin and flurbiprofen.

In another embodiment the invention provides a hybrid compound, comprising a tobramycin moiety and a nonsteroidal anti-inflammatory drug moiety selected from the group consisting of: indomethacin, flurbiprofen, and ketorolac.

In another embodiment the invention provides a hybrid compound, comprising a amikacin moiety and flurbiprofen.

In another embodiment the invention provides a pharmaceutical composition comprising a hybrid compound comprising an antibiotic moiety and a nonsteroidal anti-inflammatory drug moiety, which are connected via two separate covalent bonds to a linker such that said covalent bonds degrade in vivo to yield the antibiotic and the nonsteroidal anti-inflammatory drugs, wherein each bond is an ester bond or an amide depending on the nature of the linker, wherein said pharmaceutical composition is formulated for topical ophthalmic administration.

In another embodiment the invention provides a method comprising administrating to an eye of a mammal a hybrid compound comprising an antibiotic moiety and a nonsteroidal anti-inflammatory drug moiety, which are connected via two covalent bonds to a linker such that said hybrid compound degrades in vivo to yield the antibiotic and the nonsteroidal anti-inflammatory drugs, wherein each bond is an ester bond, wherein said method is effective in the treatment of the inflammation or bacterial infection affecting said eye.

In another embodiment the invention provides a method wherein said hybrid compound has topical antibiotic and nonsteroidal anti-inflammatory activity upon a surface of an eye, and wherein the hybrid compound degrades on said surface into said active antibiotic and said nonsteroidal anti-inflammatory drug, which are capable of penetrating beyond tissue of said surface

In another embodiment the invention provides a hybrid compound comprising a linker having two bonds, wherein said bonds are asymmetrically degraded in vivo to release the two active drugs.

Some compounds of the invention have at least one stereogenic center in their structure. This stereogenic center may be present in an R or S configuration, said R and S notation is used in correspondence with the rules described in Pure Appli. Chem. (1976), 45, 11-13.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of the invention are able to form.

The acid addition salt form of a compound of the invention that occurs in its free form as a base can be obtained by treating the free base with an appropriate acid such as an inorganic acid, for example, hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic acid and the like (Handbook of Pharmaceutical Salts, P. Heinrich Stahl & Camille G. Wermuth (Eds), Verlag Helvetica Chimica Acta—Zürich, 2002, 329-345).

The base addition salt form of a compound of the invention that occurs in its acid form can be obtained by treating the acid with an appropriate base such as an inorganic base, for example, sodium hydroxide, magnesium hydroxide, potassium hydroxide, Calcium hydroxide, ammonia and the like; or an organic base such as for example, L-Arginine, ethanolamine, betaine, benzathine, morpholine and the like. (Handbook of Pharmaceutical Salts, P. Heinrich Stahl & Camille G. Wermuth (Eds), Verlag Helvetica Chimica Acta—Zürich, 2002, 329-345).

Compounds of the invention and their salts can be in the form of a solvate, which is included within the scope of the present invention. Such solvates include for example hydrates, alcoholates and the like.

The compounds of the invention are indicated for use in treating or preventing conditions conjunctivitis, keratitis, blepharitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis.

These compounds are useful for the treatment of mammals, including humans, with a range of conditions and diseases which are alleviated by an antibiotic and NSAID drug.

In still another embodiment of the invention, there are provided methods for treating or preventing eye conditions such as: conjunctivitis, keratitis, blepharitis, endophthalmitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, in a patient suffering thereof. Such methods can be performed, for example, by administering to a subject in need thereof a therapeutically effective amount of at least one compound of the invention, or any combination thereof, or pharmaceutically acceptable salts, hydrates, solvates, crystal forms thereof.

The present invention concerns the use of a compound of the invention or a pharmaceutically acceptable salt thereof, for the manufacture of a medicament for the treatment of conjunctivitis, keratitis, blepharitis, endophthalmitis, dacyrocystitis, hordeolum, corneal ulcers, red eye, hyperemia, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

The compounds of the invention may also be administered as pharmaceutical compositions in a form suitable for topical use, for example, as oily suspensions, as solutions or suspensions in aqueous liquids or nonaqueous liquids, or as oil-in-water or water-in-oil liquid emulsions.

Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate.

A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001 to about 5 |
| preservative | 0-0.10 |
| vehicle | 0-40 |
| tonicity adjuster | 0-10 |
| buffer | 0.01-10 |
| pH adjuster | q.s. pH 4.5-7.8 |
| antioxidant | as needed |
| surfactant | as needed |
| purified water | to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses. Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

The compounds of the invention may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

The compounds and pharmaceutical compositions described herein are useful as medicaments in mammals, including humans, for treatment of diseases and/or alleviations of conditions such as conjunctivitis, keratitis, blepharitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, dry eye, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

Thus, in further embodiments of the invention, there are provided methods for treating conjunctivitis, keratitis, blepharitis, endophthalmitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, dry eye, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

Such methods can be performed, for example, by administering to a subject in need thereof a pharmaceutical composition containing a therapeutically effective amount of at least one invention compound. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

The following examples are for illustrative purposes only and are not intended, nor should they be construed as limiting the invention in any manner. Those skilled in the art will appreciate that variations and modifications of the following examples can be made without exceeding the spirit or scope of the invention.

The present invention concerns also processes for preparing the compounds of the invention. The compounds according to the invention can be prepared analogously to conventional methods as understood by the person skilled in the art of synthetic organic chemistry. Schemes 1, 2, 3 and 4 set forth below, illustrate how the compounds according to the invention can be made.

The following abbreviations are used in the examples:

CH₂Cl₂ dichloromethane
EtOH ethanol
Na₂SO₄ sodium sulfate
DMF N,N dimethylformamide
EDCI 1-ethyl-3-(3-dimethylaminopropyl)carbodiimide
THF tetrahydrofuran
BOC tert-butyl carbamate
BOC₂O di-tert-butyl pyrocarbonate
NaOH sodium hydroxide
HCl hydrochlorique acid

### DMAP 4-Dimethylaminopyridine

NaHCO₃ sodium bicarbonate
CHCl₃ chloroform
Na₂CO₃ sodium carbonate
(n-Bu)₄NHSO₄ tetrabutylammonium hydrogen sulfate

In this scheme the synthesis of hybrid compounds was started with NSAID flubioprofen. EDCI coupling with a linker gave an intermediate, which reacted with a Boc protected gatifloxacin. The removal of BOC group and fumaric acid treatment yielded the desired hybrid compound.

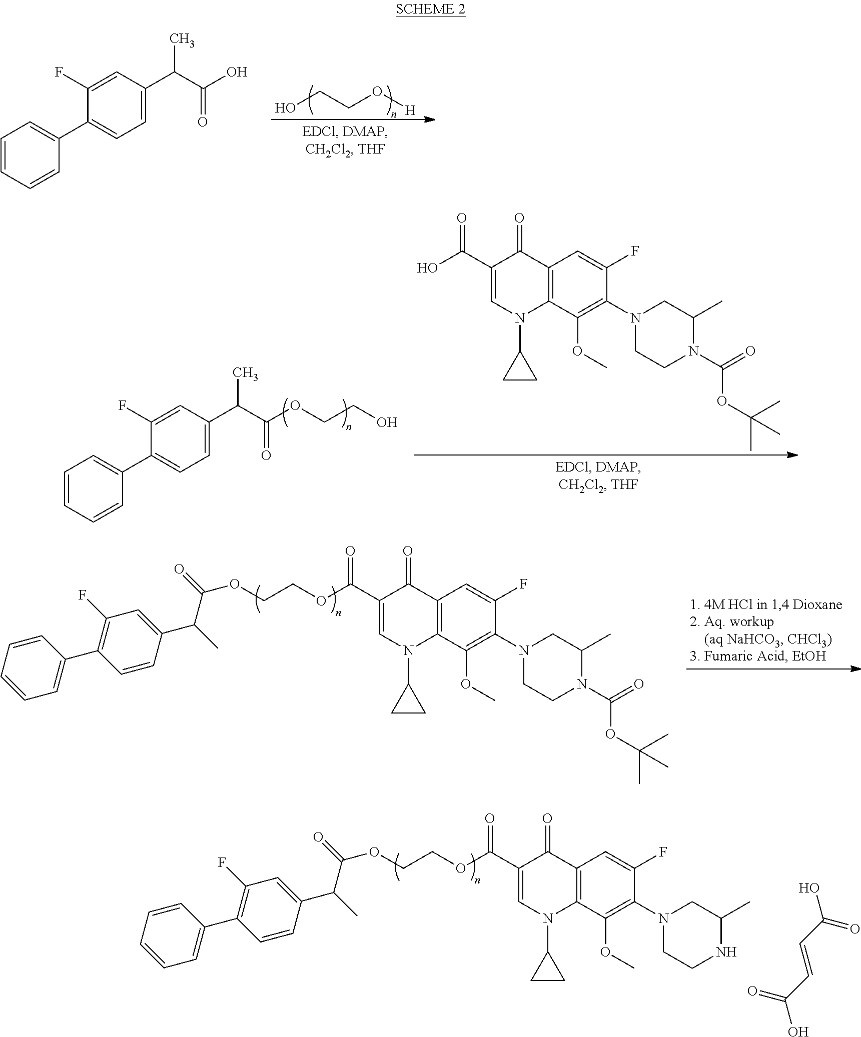

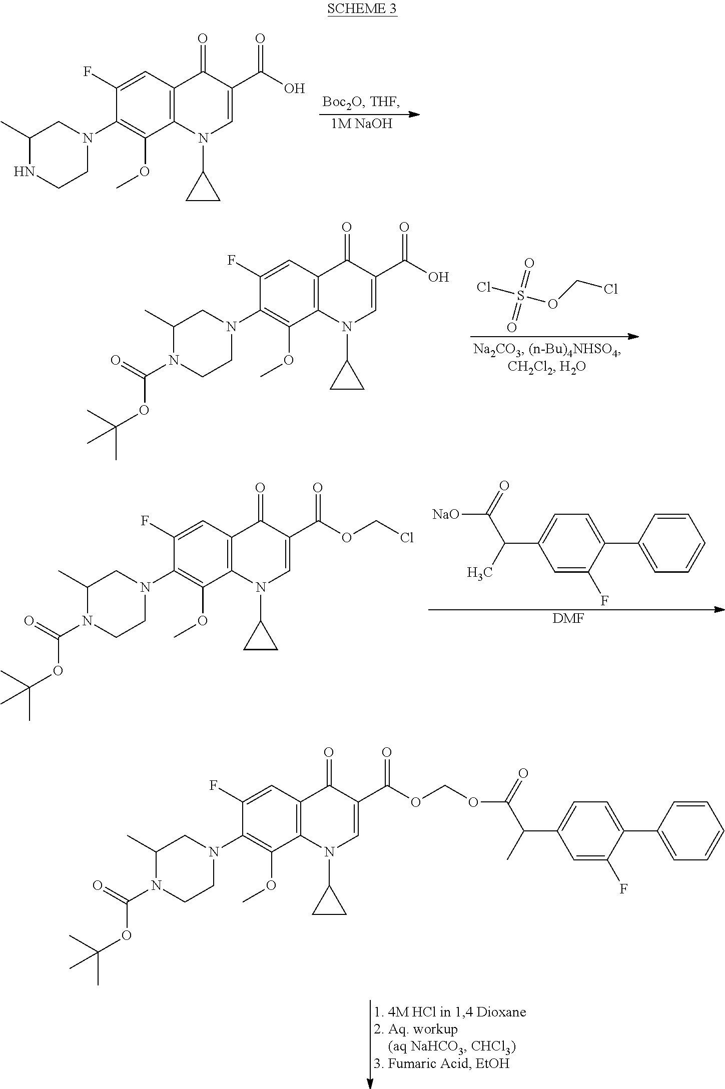

In this scheme the synthesis of hybrid analogs was started with a Boc-protected gatifloxacin. Its reaction with chloromethyl sulfochloridate gave a key intermediate chloride This intermediate reacted with the sodium salt of the steroid flubiprofen in DMF at 40° C. followed. After the removal of the BOC group and fumaric acid treatment the desired compound 16.

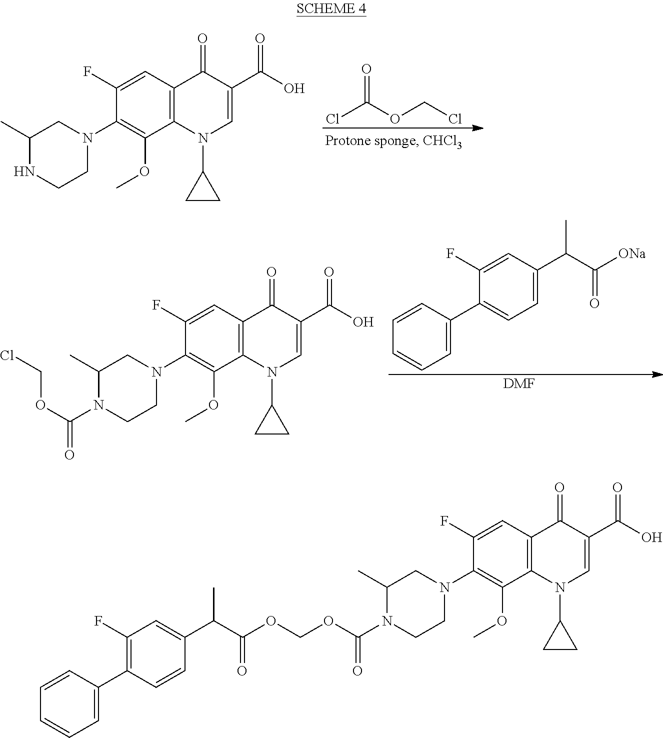

The synthesis of hybrid compounds was started with an acid intermediate acid, prepared by reaction of gatifloxacin with chloromethyl chloroformate and proton sponge in methylene chloride. Esterification with the sodium salt of flubiprofen was accomplished in DMF at 40° C. MPLC purification yielded the desired compound 16.

It is to be understood that both the foregoing general description and the following detailed description are exemplary and explanatory only and are not restrictive of the invention claimed. As used herein, the use of the singular includes the plural unless specifically stated otherwise.

It will be readily apparent to those skilled in the art that some of the compounds of the invention may contain one or more asymmetric centers, such that the compounds may exist in enantiomeric as well as in diastereomeric forms. Unless it is specifically noted otherwise, the scope of the present invention includes all enantiomers, diastereomers and racemic mixtures. Some of the compounds of the invention may form salts with pharmaceutically acceptable acids or bases, and such pharmaceutically acceptable salts of the compounds described herein are also within the scope of the invention.

The present invention includes all pharmaceutically acceptable isotopically enriched compounds. Any compound of the invention may contain one or more isotopic atoms enriched or different than the natural ratio such as deuterium ²H (or D) in place of hydrogen ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

As will be evident to those skilled in the art, individual isomeric forms can be obtained by separation of mixtures thereof in conventional manner. For example, in the case of diasteroisomeric isomers, chromatographic separation may be employed.

Compound names were generated with ACD version 12.5 or ChemBioDraw Ultra version 12.0.2. In general, characterization of the compounds is performed according to the following methods, Proton nuclear magnetic resonance (¹H NMR) and carbon nuclear magnetic resonance (¹³C NMR) spectra were recorded on a Varian 300 or 600 MHz spectrometer in deuterated solvent. Chemical shifts were reported as δ (delta) values in parts per million (ppm) relative to tetramethylsilane (TMS) as an internal standard (0.00 ppm) and multiplicities were reported as s, singlet; d, doublet; t, triplet; q, quartet; m, multiplet; br, broad. Data were reported in the following format: chemical shift (multiplicity, coupling constant(s) J in hertz (Hz), integrated intensity). The mass spectrometry data were determined on a Shimadzu LCMS-IT-TOF instrument.

The formation of the hybrid compounds was checked by ¹H-NMR by comparing the chemical shifts of protons H^{a}, H^{b} from the antibiotic molecule and of protons H^{c} and/or H^{d} of the NSAID molecule with the chemical shifts of these same protons on the newly formed hybrid molecule noted H^{a}*, H^{b}*, H^{c}* and/or H^{d}* wherein “*” indicates that the protons belong to the hybrid compound. Applicants have marked with arrows the location of these protons and the reaction site of the pro-drug moiety, where available. Each scheme shows the formation of the new hybrid drug. Each table describes the results for the new hybrid drug and the linker number, where existing. The linker and pro-drug moiety numbers are as described in Table 1 and 2 respectively.

The majority of the compounds of the invention were obtained from the synthesis between an antibiotic and a NSAID and a linker and/or a pro-drug moiety, however there are a few compounds which are obtained by linking the antibiotic directly to the NSAID.

### Examples of Compounds

Gatifloxacin reacted with flubiprofen to form the following hybrid compounds as shown in Scheme 5 with the results described in Table 4; and as shown in Scheme 6 with the results described in Table 5.

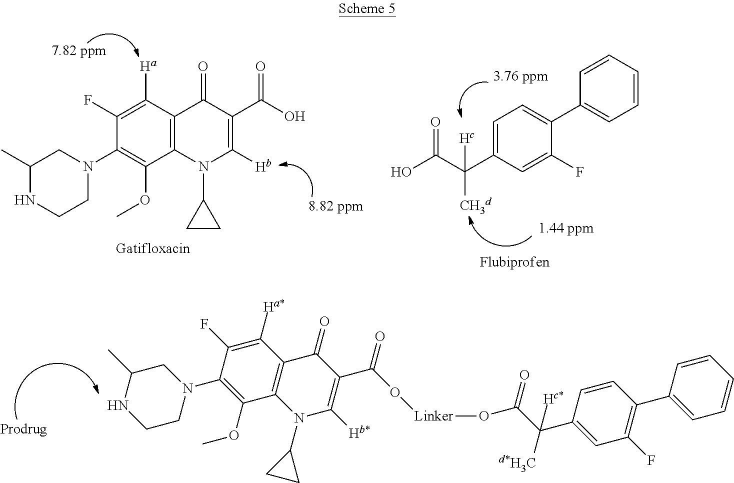

TABLE 4

| * Compound | Linker | δ ppm | | | | |
|---|---|---|---|---|---|---|
| No. | Pro-drug | H^{a}* | H^{b}* | H^{c}* | CH₃^{d}* | MASS |
| 1 | L3 | 7.84 | 8.76 | 3.78 | 1.47 | |
| 2 | L1 | 7.80 | 8.61 | 3.84 | 1.50 | 660 MH⁺ |
| 3 | L2 | 7.75 | 8.41 | 3.85 | 1.51 | 646 MH⁺ |
| 4 | L1 | 7.73 | 8.58 | 3.83 | | 772 MH⁺ |
| | P3 | | | | | |
| 5 | L2 | 7.68 | 8.37 | 3.82 | 1.50 | 756 MH⁻ |
| | P3 | | | | | |
| 6 | L3 | 7.72 | 8.68 | 3.79 | 1.46 | 847 MH+ |
| | P3 | | | | | |
| 7 | L8 | 7.82 | 8.74 | 3.81 | 1.50 | 734 MH⁺ |
| 8 | L11 | 7.83 | 8.74 | 3.83 | 1.51 | 791 MH⁺ |
| 9 | L12 | 7.78 | 8.71 | 3.88 | 1.51 | 730 MH⁺ |
| 10 | L7 | 7.75 | 8.54 | 3.88 | 1.52 | 632 MH⁺ |
| 11 | L13 | 7.76 | 8.72 | 3.77 | 1.44 | 718 MH⁺ |
| 12 | L39 | 7.83 | 8.73 | 3.81 | 1.48 | 763 MH+ |
| 13 | L29 | 7.79 | 8.72 | 3.84 | 1.49 | 816 MH+ |
| 14 | L33 | 7.80 | 8.75 | 3.82 | 1.48 | 806 MH+ |
| 82 | L41 | 7.84 | 8.85 | 3.76 | 1.49 | 674 MH+ |
| 94 | L32 | 7.84 | 8.75 | 3.82 | 1.48 | 804 MH+ |
| 95 | L29 | 7.77 | 8.75 | 3.84 | 1.46 | 814 MH+ |
| 99 | L48 | 7.83 | 8.73 | 3.81 | 1.48 | 761 MH+ |

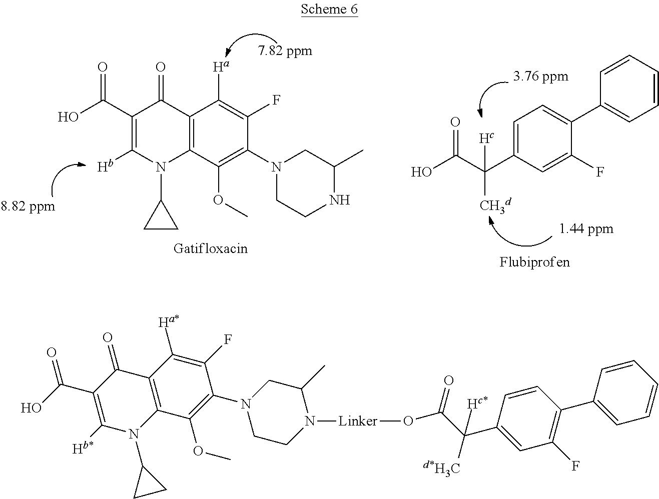

TABLE 5

| * Compound | | δ ppm | | | | |
|---|---|---|---|---|---|---|
| No. | Linker | H^{a}* | H^{b}* | H^{c}* | CH₃^{d} | MASS |
| 15 | L12 | 7.85 | 8.79 | 3.70 | 1.56 | 730 MH⁺ |
| 16 | L9 | 7.87 | 8.80 | 3.62 | 1.57 | 676 MH⁺ |
| 17 | L13 | 7.88 | 8.81 | 3.76 | 1.53 | 762 MH⁺ |
| 18 | L26 | 7.84 | 8.85 | 3.81 | 1.51 | 835 MH+ |
| 19 | L20 | 7.79 | 8.84 | 3.92 | 1.56 | 763 MH⁺ |
| 20 | L38 | 7.74 | 8.80 | 3.82 | 1.50 | 955 MNa⁺ |
| 21 | L23 | 7.79 | 8.81 | 3.76 | 1.54 | 899 MNa⁺ |
| 22 | L28 | 7.84 | 8.83 | 3.84 | 1.50 | 859 MH+ |
| 23 | L37 | 7.72 | 8.80 | 3.82 | 1.50 | 955 MNa⁺ |
| 24 | L27 | 7.81 | 8.84 | 3.83 | 1.50 | |
| 25 | L36 | 7.82 | 8.81 | 3.92 | 1.52 | |
| 26 | L25 | 7.78 | 8.89 | 3.96 | 1.47 | 689 MH⁺ |
| 27 | L30 | 7.83 | 8.85 | 3.85 | 1.51 | 850 MH⁺ |
| 28 | L34 | 7.84 | 8.85 | 3.84 | 1.51 | 834 MH+ |
| 29 | L32 | 7.84 | 8.85 | 3.84 | 1.51 | 850 MH+ |
| 83 | L42 | 7.85 | 8.78 | 3.87 | 1.51 | 838 MH+ |
| 84 | L43 | 7.84 | 8.85 | 3.85 | 1.52 | 703 MH+ |
| 85 | L44 | 7.84 | 8.85 | 3.78 | 1.48 | 790 MH+ |
| 86 | L45 | 7.82 | 8.85 | 3.76 | 1.5 | 862 MH+ |
| 87 | L32 | 7.84 | 8.85 | 3.75 | 1.51 | 848 MH+ |
| 88 | L34 | 7.82 | 8.84 | 3.75 | 1.51 | 832 MH+ |
| 89 | L30 | 7.83 | 8.85 | 3.75 | 1.51 | 848 MH+ |
| 90 | L25 | 7.78 | 8.86 | 3.96 | 1.48 | 689 MH+ |
| 91 | L46 | 7.85 | 8.80 | 3.86 | 1.50 | 811 MNa+ |
| 92 | L27 | 7.80 | 8.82 | 3.82 | 1.50 | 900 MNa+ |
| 93 | L47 | 7.85 | 8.80 | 3.82 | 1.49 | 955 MNa+ |
| 96 | L28 | 7.48 | 8.83 | 3.84 | 1.50 | 858 MH+ |

Gatifloxacin reacted with indomethacin to form the following hybrid compounds as shown in Scheme 7 with the results described in Table 6; and in Scheme 8 with the results shown in Table 7; and as shown in Scheme 29 with the results described in Table 28.

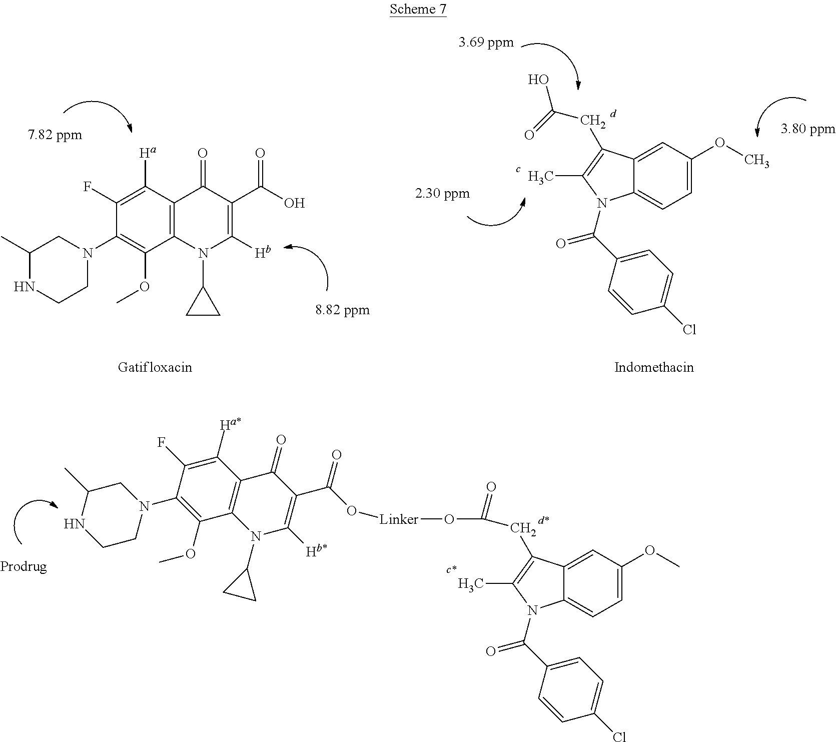

TABLE 6

| * Compound | Linker | δ ppm | | | | |
|---|---|---|---|---|---|---|
| No. | Pro-drug | H^{a}* | H^{b}* | CH₃^{c}* | CH₂^{d}* | MASS |
| 30 | L3 | 7.75 | 8.69 | 2.23 | 3.69 | 848 MH⁺ |
| 31 | L1 | 7.81 | 8.61 | 2.30 | 3.85 | 774 MH⁺ |
| 32 | L7 | 7.80 | 8.40 | 2.28 | 3.78 | 746 MH⁺ |
| 33 | L2 | 7.71 | 8.35 | 2.26 | 3.74 | 894 MNa⁺ |
| | P3 | | | | | |
| 34 | L1 | 7.74 | 8.59 | 2.308 | 3.74 | 886 MH⁺ |
| | P3 | | | | | |
| 35 | L8 | 7.64 | 8.66 | 2.21 | 3.67 | 848 MH⁺ |
| 36 | L3 | 7.65 | 8.66 | 2.24 | 3.67 | 960 MH⁺ |
| | P3 | | | | | |
| 37 | L11 | 7.81 | 8.72 | 2.27 | 3.78 | 926 MNa⁺ |
| 105 | L3 | 7.61 | 8.63 | 2.25 | 3.77 | 847 MH+ |
| 108 | L3 | 7.58 | 8.68 | 2.25 | 3.77 | 947 MH+ |

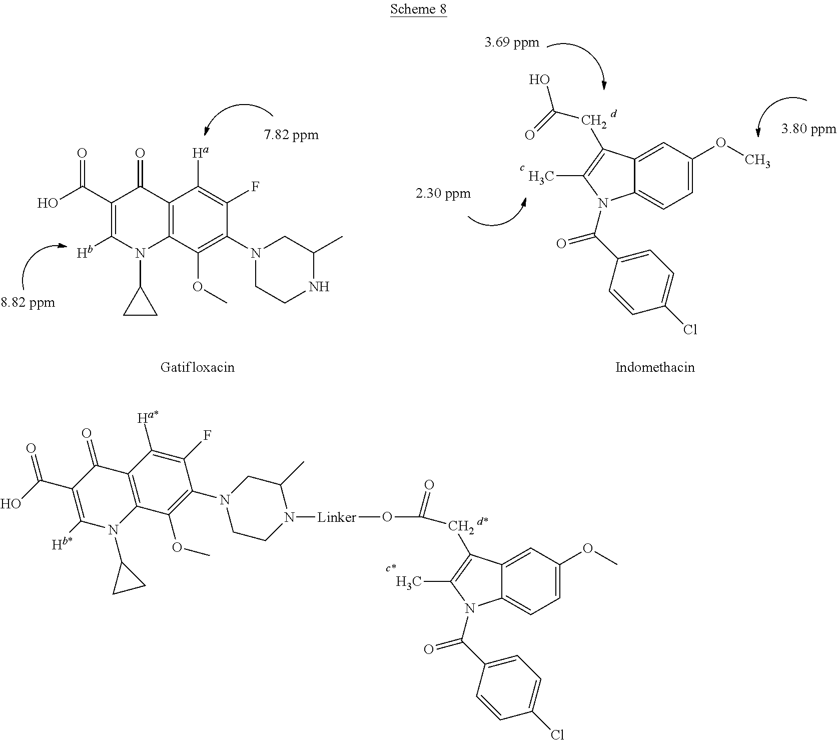

TABLE 7

| * Compound | | δ ppm | | | | |
|---|---|---|---|---|---|---|
| No. | Linker | H^{a}* | H^{b}* | CH₃^{c}* | CH₂^{d}* | MASS |
| 38 | L9 | 7.83 | 8.86 | 2.33 | 3.73 | |
| 39 | L40 | 7.78 | 8.84 | 3.82 | 3.86 | 848 MH⁺ |
| 40 | L13 | 7.88 | 8.82 | 2.38 | 3.72 | 897 MNa⁺ |

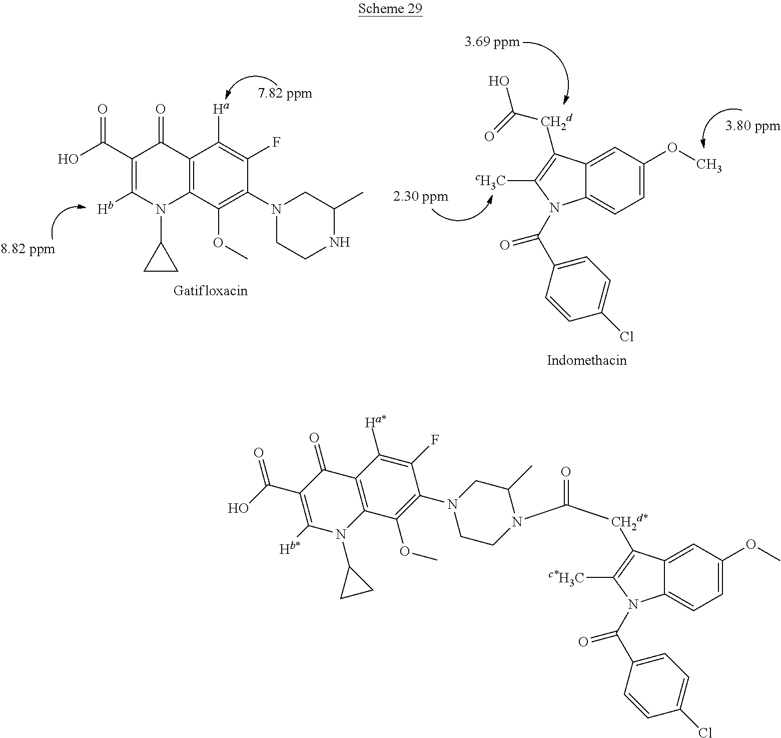

TABLE 28

| | δ ppm | | | MASS |
|---|---|---|---|---|
| * Compound No. | H^{a}* | H^{b}* | CH₂^{c}* | Mass |
| 109 | 7.85 | 8.86 | 3.80 | 715 MH⁺ |

Gatifloxacin reacted with diclofenac to form the following hybrid compounds as shown in Scheme 9 with the results described in Table 8 and in Scheme 10 with the results shown in Table 9.

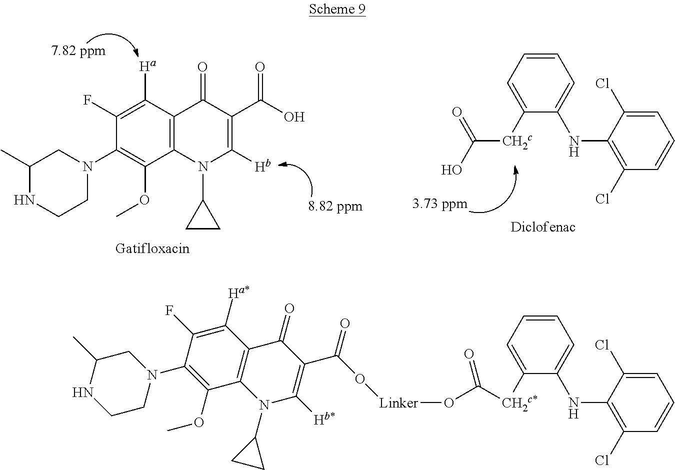

TABLE 8

| | | δ ppm | | | |
|---|---|---|---|---|---|
| * Compound No. | Linker | H^{a}* | H^{b}* | CH₂^{c}* | MASS |
| 41 | L1 | 7.84 | 8.75 | 3.82 | 712 MH⁺ |
| 42 | L7 | 7.77 | 8.60 | 3.89 | 683 MH⁺ |

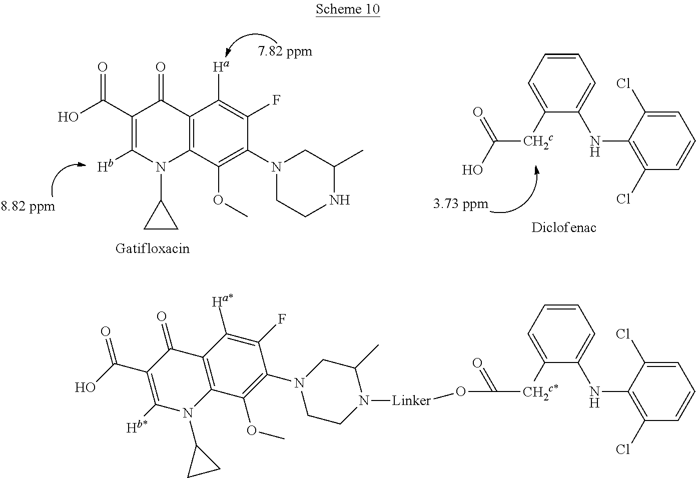

TABLE 9

| | | δ ppm | | | |
|---|---|---|---|---|---|
| * Compound No. | Linker | H^{a}* | H^{b}* | CH₂^{c}* | MASS |
| 43 | L9 | 7.88 | 8.81 | 3.89 | 727 MH⁺ |

Gatifloxacin reacted with ketorolac to form the following hybrid compounds as shown in Scheme 11 with the results described in Table 10 and in Scheme 12 with the results shown in Table 11.

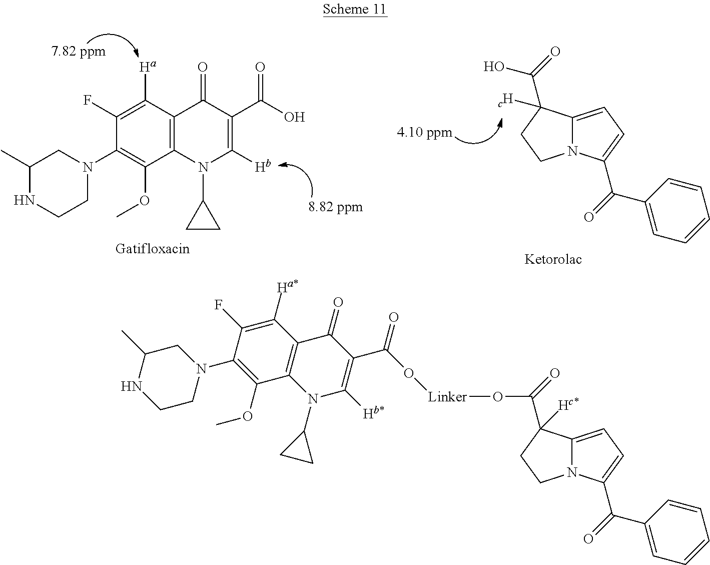

TABLE 10

| | | δ ppm | | | |
|---|---|---|---|---|---|
| * Compound No. | Linker | H^{a}* | H^{b}* | H^{c}* | MASS |
| 44 | L3 | 7.78 | 8.72 | 4.12 | 746 MH⁺ |
| 45 | L4 | 7.79 | 8.55 | 4.22 | |
| 46 | L7 | 7.82 | 8.68 | 4.20 | 743 MH⁺ |
| 47 | L11 | 7.74 | 8.74 | 4.05 | 802 MH⁺ |
| 98 | L11 | 7.74 | 8.74 | 4.05 | 800 MH+ |
| 100 | L4 | 7.75 | 8.52 | 4.20 | 755 MH+ |
| 106 | L3 | 7.73 | 8.71 | 4.05 | 844 MH+ |

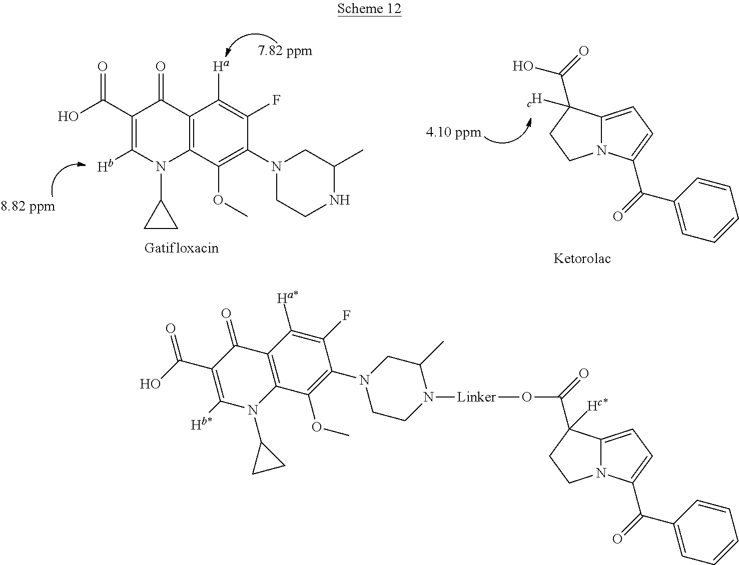

TABLE 11

| * | | | | | |
|---|---|---|---|---|---|
| Compound | | δ ppm | | | |
| No. | Linker | H^{a}* | H^{b}* | H^{c}* | MASS |
| 48 | L7 | 7.81 | 8.84 | 4.15 | 687 MH⁺ |
| 49 | L13 | 7.90 | 8.82 | 4.12 | 795 MNa⁺ |
| 50 | L26 | 7.82 | 8.84 | 3.97 | 846 MH⁺ |
| 97 | L26 | 7.82 | 8.84 | 3.97 | 844 MH⁺ |

Gatifloxacin reacted with suprofen to form the following hybrid compounds as shown in Scheme 13 with the results described in Table 12 and in Scheme 14 with the results shown in Table 13.

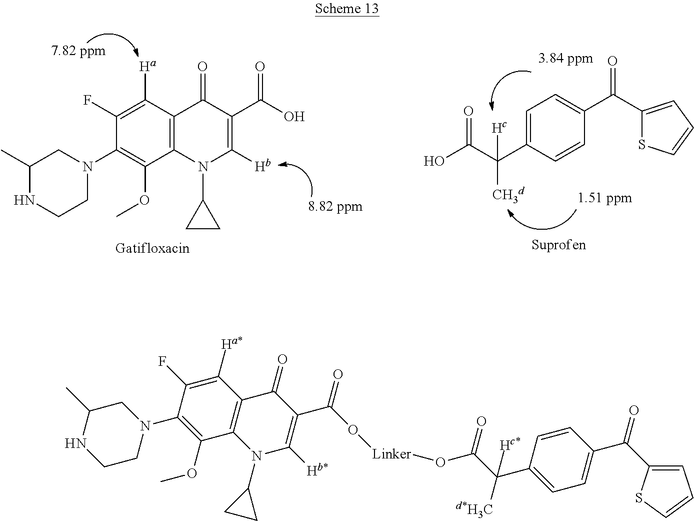

TABLE 12

| * Compound | | δ ppm | | | | |
|---|---|---|---|---|---|---|
| No. | Linker | H^{a}* | H^{b}* | H^{c}* | CH₃^{d}* | MASS |
| 51 | L7 | 7.87 | 8.47 | 3.94 | 1.54 | 648 MH⁺ |
| 52 | L11 | 7.90 | 8.72 | 3.88 | 1.51 | 807 MH⁺ |

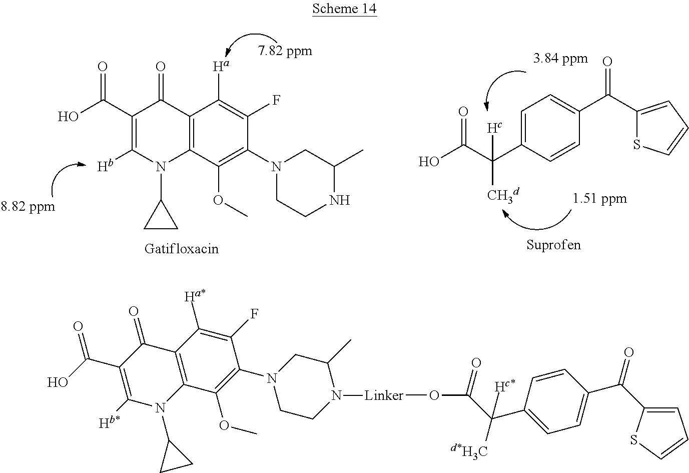

TABLE 13

| * Compound | | δ ppm | | | | |
|---|---|---|---|---|---|---|
| No. | Linker | H^{a}* | H^{b}* | H^{c}* | CH₃^{d}* | MASS |
| 53 | L9 | 7.85 | 8.81 | 3.88 | 1.59 | 714 MNa⁺ |
| 54 | L26 | 7.91 | 8.86 | 3.95 | 1.52 | 851 MH⁺ |

Moxifloxacin reacted with flubiprofen to form the following hybrid compounds as shown in Scheme 15 with the results described in Table 14 and in Scheme 16 with the results shown in Table 15.

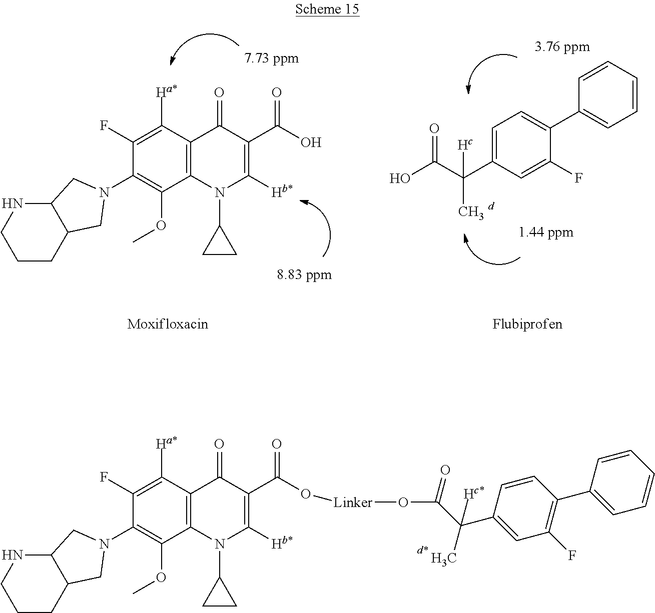

TABLE 14

| * Compound | | δ ppm | | | | |
|---|---|---|---|---|---|---|
| No. | Linker | H^{a}* | H^{b}* | H^{c}* | CH₃^{d}* | MASS |
| 55 | L2 | 7.64 | 8.40 | 3.89 | 1.51 | 673 MH⁺ |

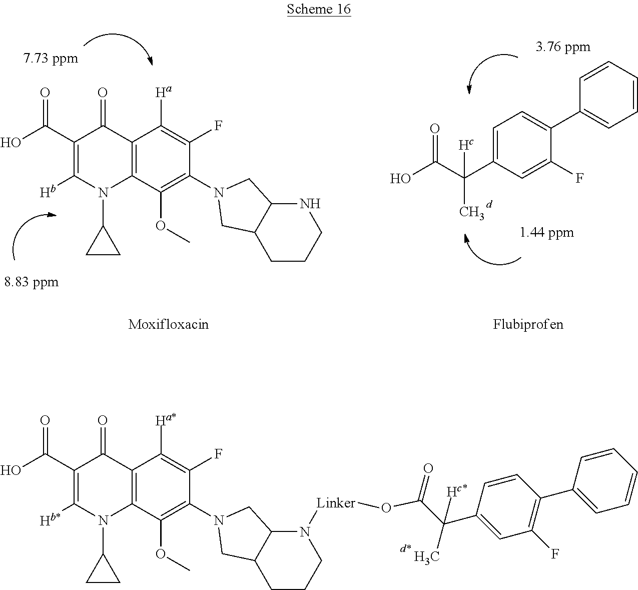

TABLE 15

| * Compound | | δ ppm | | | | |
|---|---|---|---|---|---|---|
| No. | Linker | H^{a}* | H^{b}* | H^{c}* | CH₃^{d}* | MASS |
| 56 | L9 | 7.79 | 8.76 | 3.95 | 1.55 | 724 MNa⁺ |
| 57 | L13 | 7.81 | 8.77 | 3.88 | 1.52 | 810 MNa⁺ |
| 58 | L26 | 7.72 | 8.79 | 3.81 | 1.51 | 861 MH⁺ |

Moxifloxacin reacted with indomethacin to form the following hybrid compounds as shown in Scheme 17 with the results described in Table 16 and in Scheme 18 with the results shown in Table 17.

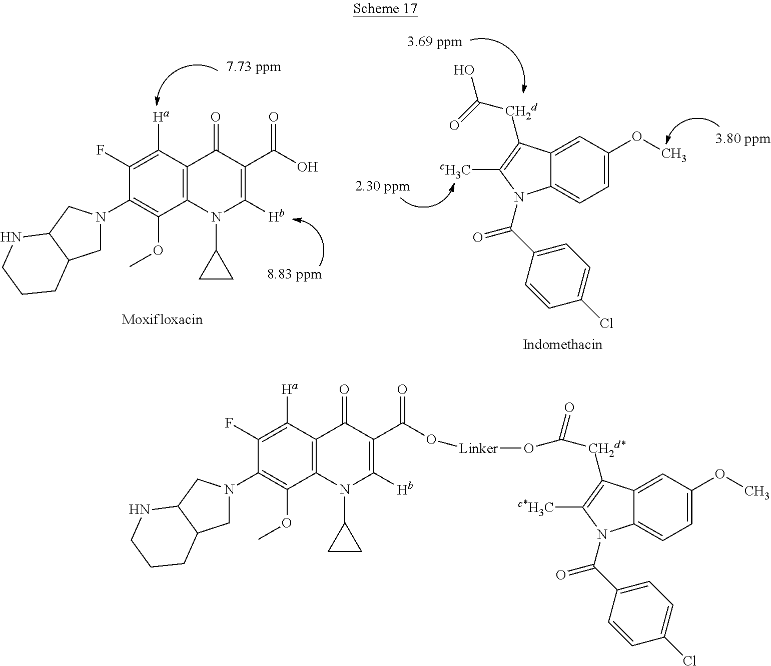

TABLE 16

| * Compound | | δ ppm | | | | |
|---|---|---|---|---|---|---|
| No. | Linker | H^{a}* | H^{b}* | CH₃^{c}* | CH₂^{d}* | MASS |
| 59 | L3 | 7.66 | 8.69 | 2.23 | 3.71 | 873 MH⁺ |
| 60 | L1 | 7.72 | 8.59 | 2.31 | 3.75 | 800 MH⁺ |
| 61 | L7 | 7.66 | 8.37 | 2.29 | 3.79 | 772 MH⁺ |
| 101 | L3 | 7.72 | 8.62 | 2.22 | 3.78 | 873 MH+ |
| 102 | L3 | 7.62 | 8.62 | 2.22 | 3.78 | 973 MH+ |

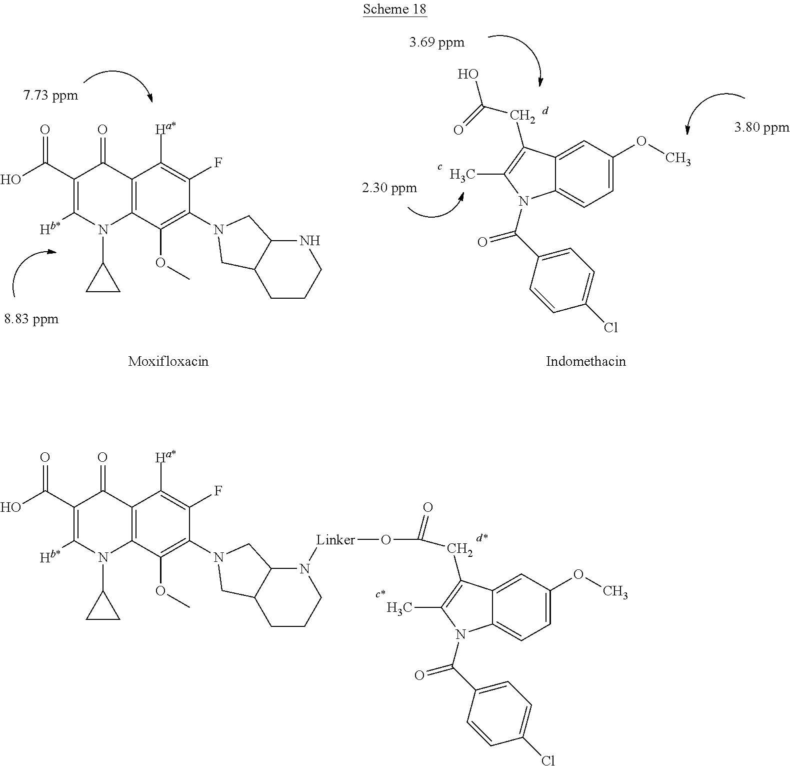

TABLE 17

| * Compound | | δ ppm | | | | |
|---|---|---|---|---|---|---|
| No. | Linker | H^{a}* | H^{b}* | CH₃^{c}* | CH₂^{d}* | MASS |
| 62 | L9 | 7.80 | 8.77 | 2.37 | 3.72 | 837 MNa⁺ |
| 63 | L13 | 7.79 | 8.78 | 2.38 | 3.65 | 901 MH⁺ |

Moxifloxacin reacted with diclofenac to form the following hybrid compounds as shown in Scheme 19 with the results described in Table 18.

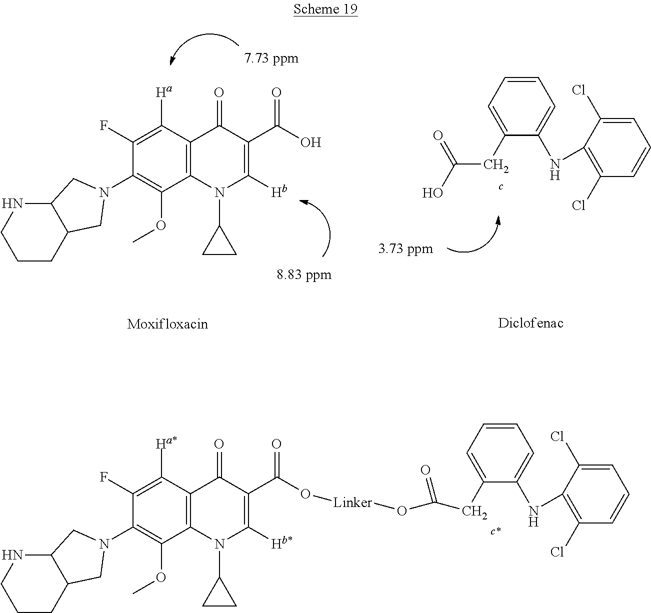

TABLE 18

| * | | | | | |
|---|---|---|---|---|---|
| Compound | | δ ppm | | | |
| No. | Linker | H^{a}* | H^{b}* | CH₂^{c}* | MASS |
| 64 | L1 | 7.61 | 8.65 | 3.82 | 738 MH⁺ |

Moxifloxacin reacted with ketorolac form the following hybrid compounds as shown in Scheme 20 with the results described in Table 19 and in Scheme 21 with the results shown in Table 20.

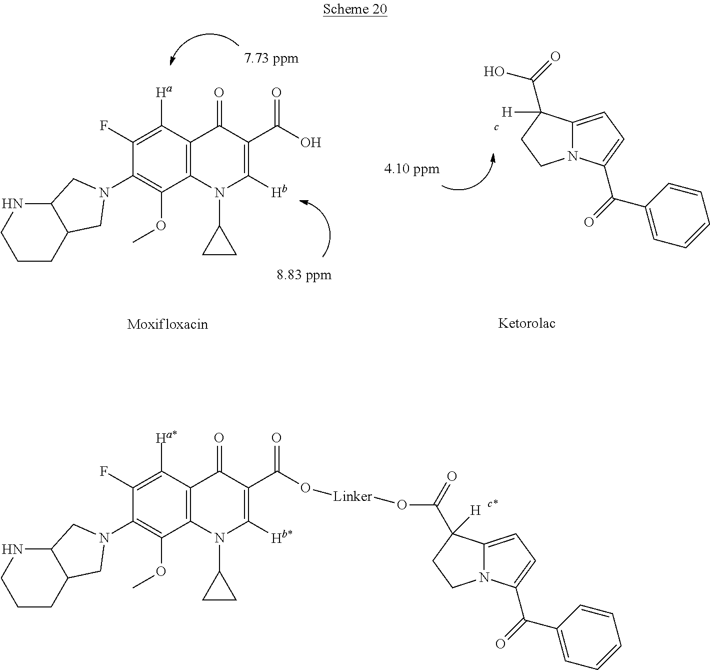

TABLE 19

| * | | | | | |
|---|---|---|---|---|---|
| Compound | | δ ppm | | | |
| No. | Linker | H^{a}* | H^{b}* | H^{c}* | MASS |
| 65 | L3 | 7.71 | 8.73 | 4.12 | 772 MH⁺ |
| 66 | L4 | 7.72 | 8.80 | 4.02 | |
| 103 | L3 | 7.71 | 8.70 | 4.01 | 770 MH⁺ |

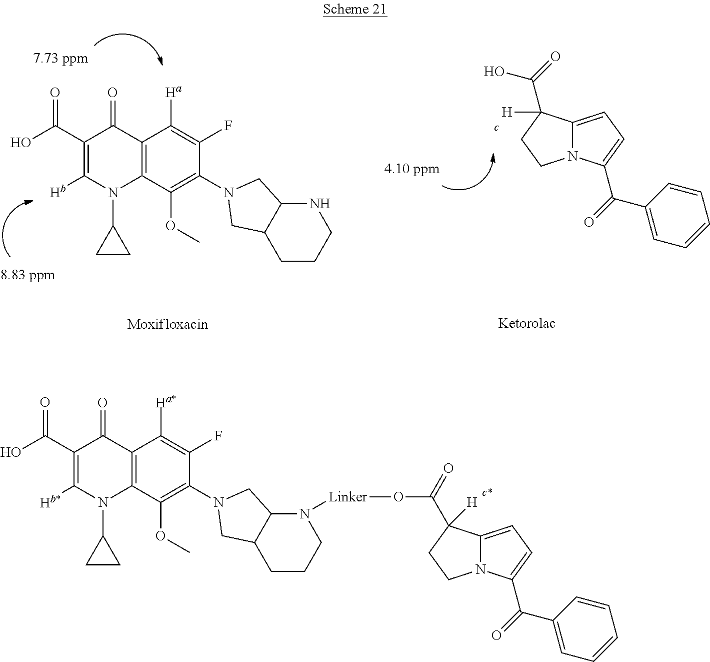

TABLE 20

| * | | | | | |
|---|---|---|---|---|---|
| Compound | | δ ppm | | | |
| No. | Linker | H^{a}* | H^{b}* | H^{c}* | MASS |
| 67 | L9 | 7.75 | 8.78 | 4.25 | 713 MH⁺ |
| 68 | L13 | 7.79 | 8.77 | 4.22 | 821 MNa⁺ |

Moxifloxacin reacted with suprofen form the following hybrid compounds as shown in Scheme 22 with the results described in Table 21.

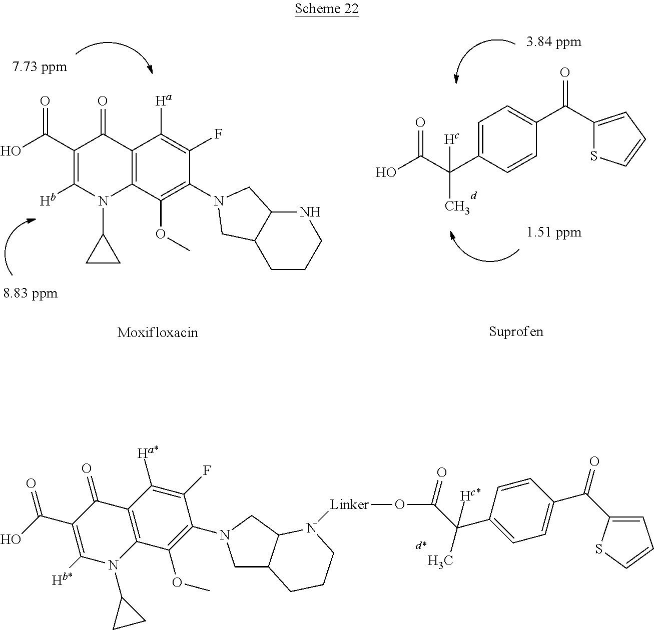

TABLE 21

| * Compound | | δ ppm | | | | |
|---|---|---|---|---|---|---|
| No. | Linker | H^{a}* | H^{b}* | H^{c}* | CH₃^{d}* | MASS |
| 69 | L26 | 7.90 | 8.66 | 3.88 | 1.52 | 876 MH⁺ |

Chloramphenicol reacted with indomethacin form the following hybrid compounds as shown in Scheme 23 with the results described in Table 22; as shown in Scheme 30 with the results described in Table 29.

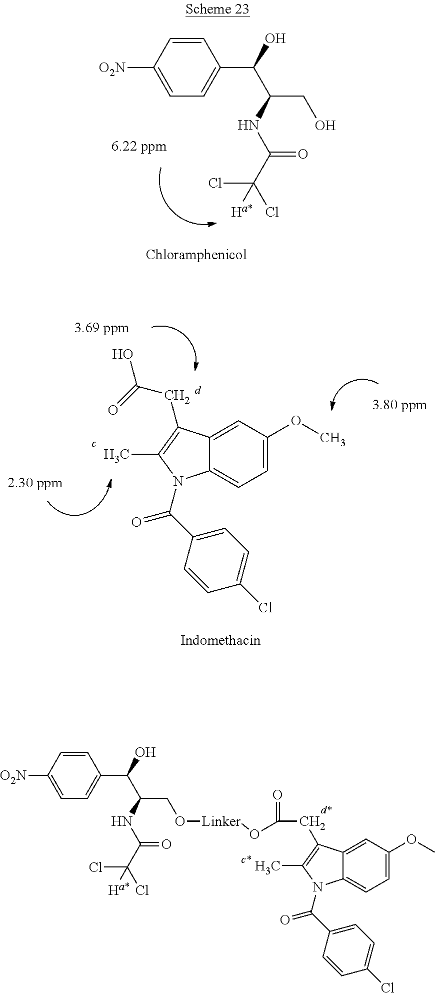

TABLE 22

| * | | | | | |
|---|---|---|---|---|---|
| Compound | | δ ppm | | | |
| No. | Linker | H^{a}* | CH₃^{c*} | CH₂^{d}* | MASS |
| 70 | L6 | 6.17 | 2.31 | 3.76 | 750 MH⁺ |
| 71 | L5 | 6.17 | 2.35 | 3.64 | 750 MH⁺ |
| 73 | L35 | 6.16 | 2.30 | 3.85 | 743 MNa⁺ |

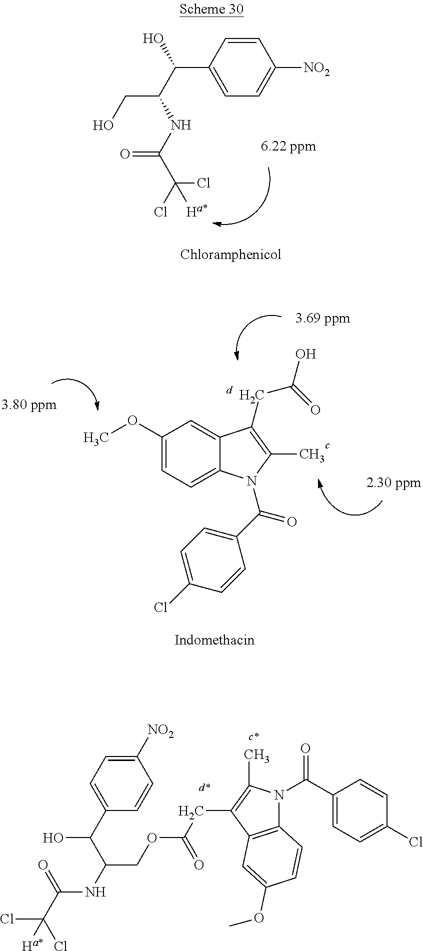

TABLE 29

| * | | | | |
|---|---|---|---|---|
| Compound | δ ppm | | | |
| No. | H^{a}* | CH₃^{c}* | CH₂^{d}* | MASS |
| 72 | 6.08 | 2.30 | 3.78 | 664 MH⁺ |

Chloramphenicol reacted with flubiprofen form the following hybrid compounds as shown in Scheme 24 with the results shown in Table 23.

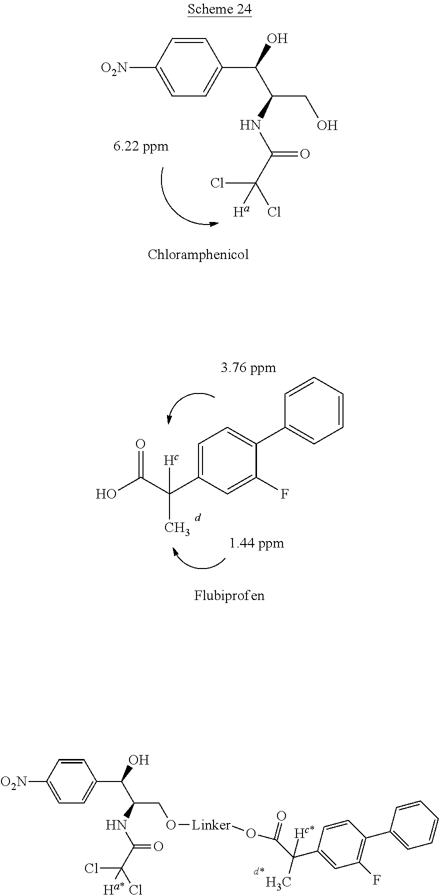

TABLE 23

| * | | | | | |
|---|---|---|---|---|---|
| Compound | | δ ppm | | | |
| No. | Linker | H^{a}* | H^{c}* | CH₃^{d}* | MASS |
| 74 | L5 | 6.17 | 3.82 | 1.44 | 634 MH⁺ |

Tobramycin reacted with indomethacin form the following hybrid compounds as shown in Scheme 25 with the results shown in Table 24; and as shown in Scheme 31 with the results shown in Table 30.

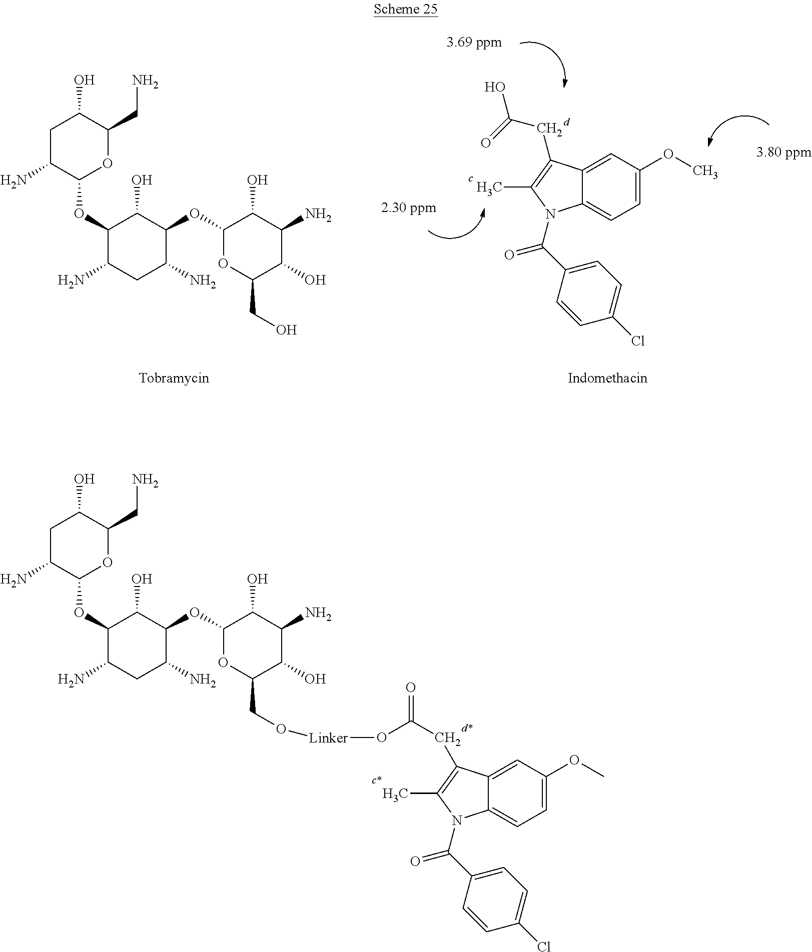

TABLE 24

| * | | | | |
|---|---|---|---|---|
| Compound | | δ ppm | | |
| No. | Linker | CH₃^{c}* | CH₂^{d}* | MASS |
| 75 | L5 | 2.36 | 3.75 | 916 MNa⁺ |

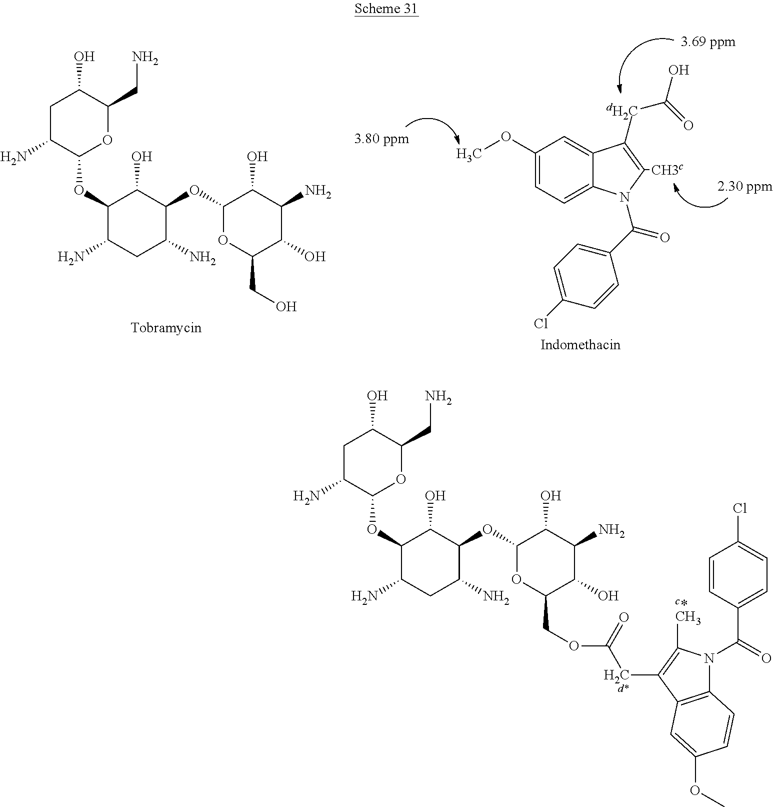

TABLE 30

| * | | | |
|---|---|---|---|
| Compound | δ ppm | | |
| No. | CH₃^{c}* | CH₂^{d}* | MASS |
| 78 | 2.40 | 3.78 | 807 MH⁺ |

Tobramycin reacted with ketorolac and formed the following hybrid compounds as shown in Scheme 26 with the results shown in Table 25.

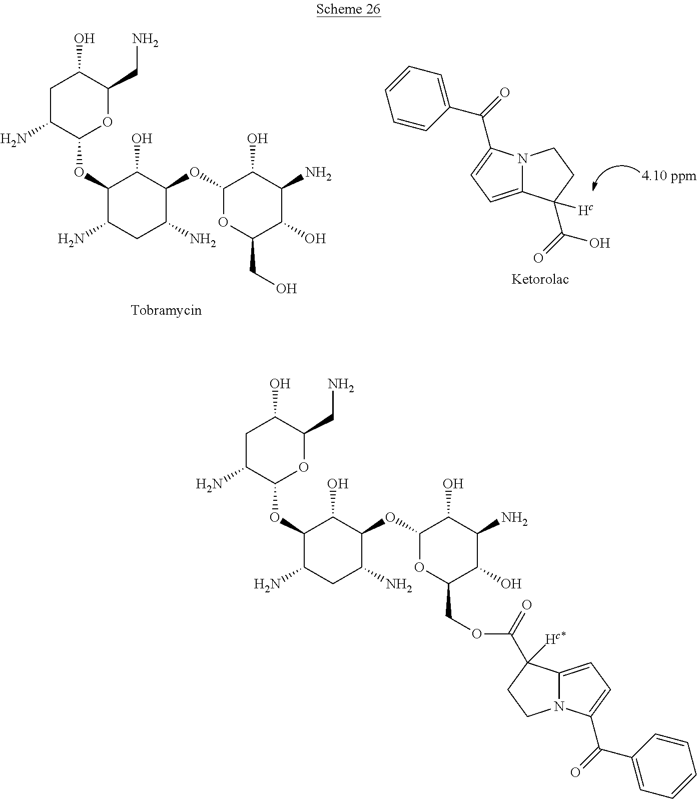

TABLE 25

| * | | |
|---|---|---|
| Compound | δ ppm | |
| No. | H^{c}* | MASS |
| 79 | 4.22 | 727 MNa⁺ |

Tobramycin reacted with flubiprofen and formed the following hybrid compounds as shown in Scheme 27 with the results shown in Table 26; and as shown in Scheme 32 with the results shown in Table 31.

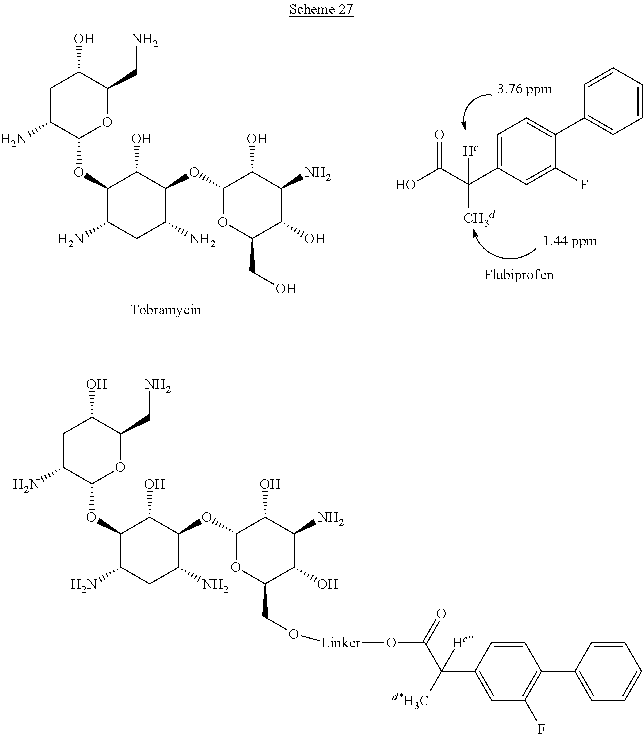

TABLE 26

| * | | | | |
|---|---|---|---|---|
| Compound | | δ ppm | | |
| No. | Linker | H^{c}* | CH₃^{d}* | MASS |
| 76 | L5 | 3.83 | 1.50 | 780 MH⁺ |

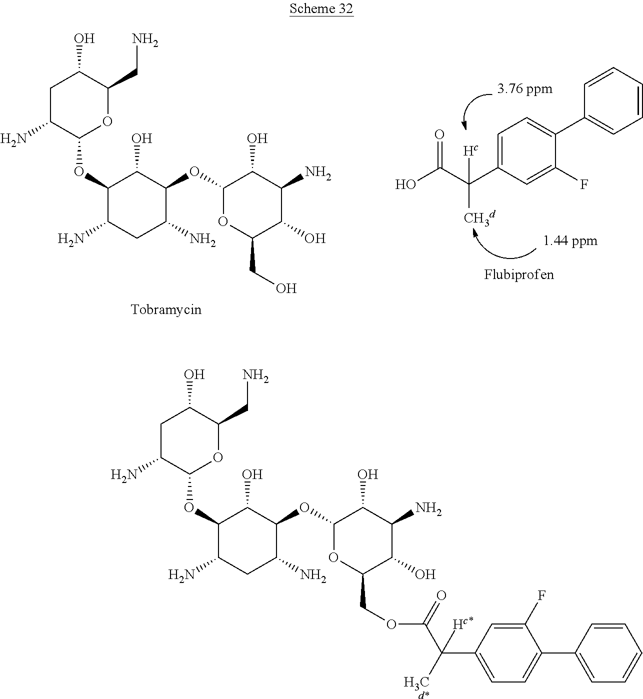

TABLE 31

| * | | | |
|---|---|---|---|
| Compound | δ ppm | | |
| No. | H^{c}* | CH₃^{d}* | MASS |
| 80 | 3.87 | 1.52 | 716 MNa⁺ |

Amikacin reacted with flubiprofen and formed the following hybrid compounds as shown in Scheme 28 with the results shown in Table 27.

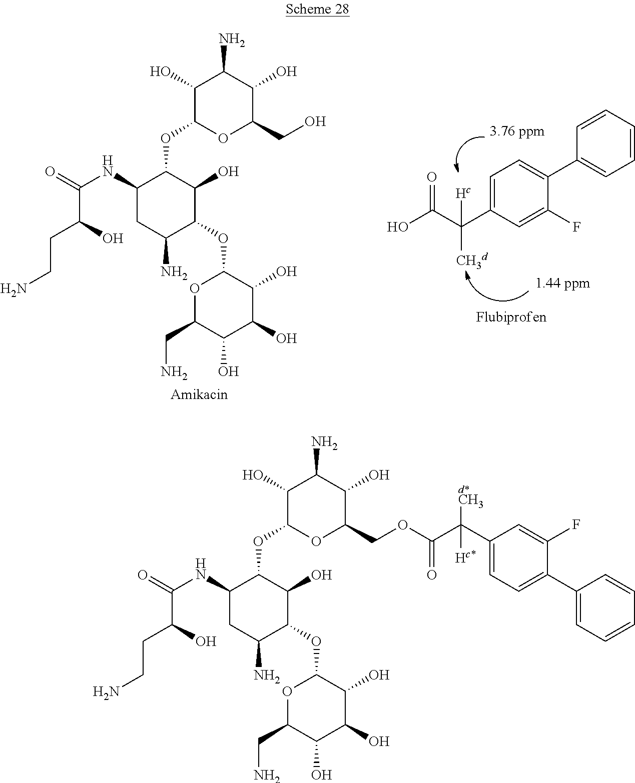

TABLE 27

| * | | | |
|---|---|---|---|
| Compound | δ ppm | | |
| No. | H^{c}* | CH₃^{d}* | MASS |
| 81 | 3.85 | 1.52 | 834 MNa⁺ |

### Biological Examples

### Example 1

### In Vitro Metabolic Stability in Rabbit Cornea Homogenates and Human Recombinant Carboxylesterases

Dutch Belted rabbits were euthanized with an overdose of sodium pentobarbital. The corneas were collected and homogenized in ice-cold potassium chloride solution (pH=7.4). The homogenate was centrifuged at 755×g for 30 min at 4° C. and aliquots of the supernatant were stored at or below −70° C. until metabolism experiments were conducted. Prior to storing the homogenates an aliquot was removed for determination of protein concentrations by calculating the 260 nm absorbance using a spectrophotometer. Human recombinant carboxylesterases were purchased from a commercial vendor (BD Gentest™, Bedford, Mass.)

All metabolic stability experiments were performed in triplicate in 96-well plate format. The final incubation mixture contained 1 μM test compound, 0.3 mg/mL corneal protein homogenate or 0.1 mg/mL human recombinant carboxylesterase mixture in a final volume of 0.5 mL 0.1 M potassium phosphate buffer (pH=6.0). The final percentage of solvent in the incubation was less than 1.0% to prevent inhibition of enzymatic activity. Following a pre-incubation at 37° C., test article (i.e. ester linked hybrids) was added to initiate the reaction. At designated time points (typically less than 60 minutes to capture the linear range of metabolite formation), 0.05 mL aliquots were removed from the incubation mixtures using a clean pipette tip and immediately placed in organic solvent to stop any esterase activity. Hydrolysis to the metabolites was proved to be due to esterase activity and not to chemical liability.

The samples were analyzed by liquid chromatography with mass spectrometry (LC-MS/MS) detection to determine the metabolite concentrations resulting from the metabolism of ester linked hybrids. Internal standards were used to compensate for variability from sample processing, chromatographic elution, mass spectrometer response and ion suppression by matrix components.

### Results

Table 28 lists the rate of metabolite formation in rabbit cornea homogenates

TABLE 28

| | | Rate of | Rate of |
|---|---|---|---|
| * | | formation | formation |
| Com- | | Metabolite 1 | Metabolite 2 |
| pound | IUPAC name | (nM/min/mg) | (nM/min/mg) |
| 2 | 3-{[2-(2-fluorobiphenyl-4- | 3.38 ± 1.50 | 45.7 ± 5.3 |
| | yl)propanoyl]oxy}propyl 1- | Gatifloxacin | Flurbiprofen |
| | cyclopropyl-6-fluoro-8-methoxy-7- | | |
| | (3-methylpiperazin-1-yl)-4-oxo-1,4- | | |
| | dihydroquinoline-3-carboxylate | | |

Table 29 lists the rate of metabolite formation in human recombinant carboxylesterases

TABLE 29

| | | Rate of | Rate of |
|---|---|---|---|
| * | | formation | formation |
| Com- | | Metabolite | Metabolite |
| pound | | 1 (nM/ | 2 (nM/ |
| number | IUPAC name | min/mg) | min/mg) |
| 2 | 3-{[2-(2-fluorobiphenyl-4- | 4.67 ± 2.55 | 573 ± 68 |
| | yl)propanoyl]oxy}propyl 1- | Gatifloxacin | Flurbiprofen |
| | cyclopropyl-6-fluoro-8-methoxy- | | |
| | 7-(3-methylpiperazin-1-yl)-4-oxo- | | |
| | 1,4-dihydroquinoline-3-carboxylate | | |
| 38 | 7-[4-({[({1-[(4-chlorophenyl) | 535 ± 28 | 191 ± 95 |
| | carbonyl]-5-methoxy-2-methyl-1H- | Indomethacin | Gatifloxacin |
| | indol-3-yl}acetyl)oxy]methoxy} | | |
| | carbonyl)-3-methylpiperazin-1-yl]- | | |
| | 1-cyclopropyl-6-fluoro-8-methoxy- | | |
| | 4-oxo-1,4-dihydroquinoline-3- | | |
| | carboxylic acid | | |
| 4 | 3-{[2-(2-fluorobiphenyl-4-yl) | 1590 ± 90 | 186 ± 15 |
| | propanoyl]oxy}propyl 1- | Flurbiprofen | Gatifloxacin |
| | cyclopropyl-6-fluoro-8-methoxy- | | |
| | 7-{3-methyl-4-[(5-methyl-2-oxo- | | |
| | 1,3-dioxol-4-yl)methyl] | | |
| | piperazin-1-yl}-4-oxo-1,4- | | |
| | dihydroquinoline-3-carboxylate | | |
| 6 | 2-[2-(2-{[2-(2-fluorobiphenyl-4- | 157 ± 9 | 12.8 ± 1.2 |
| | yl)propanoyl]oxy}ethoxy)ethoxy] | Flurbiprofen | Gatifloxacin |
| | ethyl 1-cyclopropyl-6-fluoro-8- | | |
| | methoxy-7-{3-methyl-4-[(5- | | |
| | methyl-2-oxo-1,3-dioxol-4-yl) | | |
| | methyl]piperazin-1-yl}-4-oxo-1,4- | | |
| | dihydroquinoline-3-carboxylate | | |
| 5 | 2-{[2-(2-fluorobiphenyl-4-yl) | 94.7 ± 8.8 | 11.6 ± 1.3 |
| | propanoyl]oxy}ethyl 1- | Flurbiprofen | Gatifloxacin |
| | cyclopropyl-6-fluoro-8-methoxy- | | |
| | 7-{3-methyl-4-[(5-methyl-2-oxo- | | |
| | 1,3-dioxol-4-yl)methyl]piperazin- | | |
| | 1-yl}-4-oxo-1,4-dihydroquinoline- | | |
| | 3-carboxylate | | |
| 35 | [({3-[({1-[(4-chlorophenyl) | 234 ± 10 | 1900 ± 300 |
| | carbonyl]-5-methoxy-2-methyl- | Indomethacin | Gatifloxacin |
| | 1H-indol-3-yl}acetyl)oxy] | | |
| | propoxy}carbonyl)oxy]methyl | | |
| | 1-cyclopropyl-6-fluoro-8- | | |
| | methoxy-7-(3-methylpiperazin-1- | | |
| | yl)-4-oxo-1,4-dihydroquinoline- | | |
| | 3-carboxylate | | |
| 7 | {[(3-{[2-(2-fluorobiphenyl-4-yl) | 92.8 ± 15.0 | 143 ± 31 |
| | propanoyl]oxy}propoxy)carbonyl] | Flurbiprofen | Gatifloxacin |
| | oxy}methyl 1-cyclopropyl-6- | | |
| | fluoro-8-methoxy-7-(3- | | |
| | methylpiperazin-1-yl)-4-oxo-1,4- | | |
| | dihydroquinoline-3-carboxylate | | |
| 37 | 3-[({1-[(4-chlorophenyl)carbonyl]- | 204 ± 10 | 970 ± 190 |
| | 5-methoxy-2-methyl-1H-indol-3- | Indomethacin | Gatifloxacin |
| | yl}acetyl)oxy]propyl ({[1- | | |
| | cyclopropyl-6-fluoro-8-methoxy- | | |
| | 7-(3-methylpiperazin-1-yl)-4-oxo- | | |
| | 1,4-dihydroquinolin-3-yl]carbonyl} | | |
| | oxy)methyl butanedioate | | |
| 8 | ({[1-cyclopropyl-6-fluoro-8- | 569 ± 109 | 543 ± 96 |
| | methoxy-7-(3-methylpiperazin-1- | Flurbiprofen | Gatifloxacin |
| | yl)-4-oxo-1,4-dihydroquinolin-3- | | |
| | yl]carbonyl}oxy)methyl 3- | | |
| | {[2-(2-fluorobiphenyl-4-yl) | | |
| | propanoyl]oxy}propyl | | |
| | butanedioate | | |
| 9 | [5-({[2-(2-fluorobiphenyl-4-yl) | 219 ± 50 | 470 ± 160 |
| | propanoyl]oxy}methyl)-2-oxo-1,3- | Flurbiprofen | Gatifloxacin |
| | dioxol-4-yl]methyl 1-cyclopropyl- | | |
| | 6-fluoro-8-methoxy-7-(3- | | |
| | methylpiperazin-1-yl)-4-oxo-1,4- | | |
| | dihydroquinoline-3-carboxylate | | |
| 16 | 1-cyclopropyl-6-fluoro-7-{4-[({[2- | 1380 ± 290 | 1250 ± 290 |
| | (2-fluorobiphenyl-4-yl)propanoyl] | Flurbiprofen | Gatifloxacin |
| | oxy}methoxy)carbonyl]-3- | | |
| | methylpiperazin-1-yl}-8-methoxy- | | |
| | 4-oxo-1,4-dihydroquinoline-3- | | |
| | carboxylic acid | | |
| 17 | 1-cyclopropyl-6-fluoro-7-[4- | 545 ± 102 | 333 ± 194 |
| | ({[(4-{[2-(2-fluorobiphenyl-4-yl) | Flurbiprofen | Gatifloxacin |
| | propanoyl]oxy}butanoyl)oxy] | | |
| | methoxy}carbonyl)-3- | | |
| | methylpiperazin-1-yl]-8- | | |
| | methoxy-4-oxo-1,4- | | |
| | dihydroquinoline-3-carboxylic | | |
| | acid | | |
| 53 | 1-cyclopropyl-6-fluoro-8-methoxy- | 564 ± 96 | 498 ± 108 |
| | 7-(3-methyl-4-{[({2-[4-(thiophen- | Suprofen | Gatifloxacin |
| | 2-ylcarbonyl)phenyl]propanoyl} | | |
| | oxy)methoxy]carbonyl}piperazin- | | |
| | 1-yl)-4-oxo-1,4-dihydroquinoline- | | |
| | 3-carboxylic acid | | |
| 51 | ({2-[4-(thiophen-2-ylcarbonyl) | 340 ± 76 | 352 ± 82 |
| | phenyl]propanoyl}oxy)methyl 1- | Suprofen | Gatifloxacin |
| | cyclopropyl-6-fluoro-8-methoxy-7- | | |
| | (3-methylpiperazin-1-yl)-4-oxo- | | |
| | 1,4-dihydroquinoline-3-carboxylate | | |
| 56 | 1-cyclopropyl-6-fluoro-7-{1-[({[2- | 702 ± 100 | 768 ± 200 |
| | (2-fluorobiphenyl-4-yl) | Flurbiprofen | Moxifloxacin |
| | propanoyl]oxy}methoxy)carbonyl] | | |
| | octahydro-6H-pyrrolo[3,4-b] | | |
| | pyridin-6-yl}-8-methoxy-4-oxo- | | |
| | 1,4-dihydroquinoline-3- | | |
| | carboxylic acid | | |
| 62 | 7-[1-({[({1-[(4-chlorophenyl) | 512 ± 48 | 588 ± 56 |
| | carbonyl]-5-methoxy-2-methyl-1H- | Indomethacin | Moxifloxacin |
| | inden-3-yl}acetyl)oxy]methoxy} | | |
| | carbonyl)octahydro-6H-pyrrolo | | |
| | [3,4-b]pyridin-6-yl]-1-cyclopropyl- | | |
| | 6-fluoro-8-methoxy-4-oxo-1,4- | | |
| | dihydroquinoline-3-carboxylic acid | | |
| 12 | ({[1-cyclopropyl-6-fluoro-8- | 129 ± 89 | 282 ± 152 |
| | methoxy-7-(3-methylpiperazin-1- | Flurbiprofen | Gatifloxacin |
| | yl)-4-oxo-1,4-dihydroquinolin-3- | | |
| | yl]carbonyl}oxy)methyl | | |
| | {[2-(2-fluorobiphenyl-4-yl) | | |
| | propanoyl]oxy}methyl | | |
| | butanedioate | | |
| 57 | 1-cyclopropyl-6-fluoro-7-[1-({[(4- | 146 ± 26 | 243 ± 54 |
| | {[2-(2-fluorobiphenyl-4- | Flurbiprofen | Moxifloxacin |
| | yl)propanoyl]oxy}butanoyl)oxy] | | |
| | methoxy}carbonyl)octahydro-6H- | | |
| | pyrrolo[3,4-b]pyridin-6-yl]-8- | | |
| | methoxy-4-oxo-1,4- | | |
| | dihydroquinoline-3-carboxylic | | |
| | acid | | |
| 49 | 1-cyclopropyl-6-fluoro-8-methoxy- | 27.1 ± 5.7 | 14.3 ± 12.1 |
| | 7-{3-methyl-4-[({[4-({[5- | Ketorolac | Gatifloxacin |
| | (phenylcarbonyl)-2,3-dihydro-1H- | | |
| | pyrrolizin-1-yl]carbonyl}oxy) | | |
| | butanoyl]oxy}methoxy)carbonyl] | | |
| | piperazin-1-yl}-4-oxo-1,4- | | |
| | dihydroquinoline-3-carboxylic | | |
| | acid | | |
| 54 | 1-cyclopropyl-6-fluoro-8-methoxy- | 22.7 ± 10.4 | 33.1 ± 3.8 |
| | 7-(3-methyl-4-{5,8,14-trioxo-15- | Suprofen | Gatifloxacin |
| | [4-(thiophen-2-ylcarbonyl)phenyl]- | | |
| | 2,4,9,13-tetraoxahexadecan-1-oyl} | | |
| | piperazin-1-yl)-4-oxo-1,4- | | |
| | dihydroquinoline-3-carboxylic | | |
| | acid | | |
| 52 | ({[1-cyclopropyl-6-fluoro-8- | 36.3 ± 3.5 | 42.3 ± 11.1 |
| | methoxy-7-(3-methylpiperazin-1- | Suprofen | Gatifloxacin |
| | yl)-4-oxo-1,4-dihydroquinolin-3- | | |
| | yl]carbonyl}oxy)methyl 3-({2- | | |
| | [4-(thiophen-2-ylcarbonyl) | | |
| | phenyl]propanoyl}oxy)propyl | | |
| | butanedioate | | |
| 48 | 1-cyclopropyl-6-fluoro-8-methoxy- | 6.52 ± 4.17 | 18.5 ± 2.9 |
| | 7-(3-methyl-4-{[({[5- | Ketorolac | Gatifloxacin |
| | (phenylcarbonyl)-2,3-dihydro- | | |
| | 1H-pyrrolizin-1-yl]carbonyl}oxy) | | |
| | methoxy]carbonyl}piperazin-1-yl)- | | |
| | 4-oxo-1,4-dihydroquinoline-3- | | |
| | carboxylic acid | | |
| 47 | ({[1-cyclopropyl-6-fluoro-8- | 23.1 ± 3.4 | 20.6 ± 3.1 |
| | methoxy-7-(3-methylpiperazin-1- | Ketorolac | Gatifloxacin |
| | yl)-4-oxo-1,4-dihydroquinolin-3- | | |
| | yl]carbonyl}oxy)methyl 3-({[5- | | |
| | (phenylcarbonyl)-2,3-dihydro-1H- | | |
| | pyrrolizin-1-yl]carbonyl}oxy) | | |
| | propyl butanedioate | | |
| 50 | 1-cyclopropyl-6-fluoro-8-methoxy- | 56.5 ± 3.6 | 85.3 ± 12.0 |
| | 7-(3-methyl-4-{5,8,14-trioxo-14- | Ketorolac | Gatifloxacin |
| | [5-(phenylcarbonyl)-2,3-dihydro- | | |
| | 1H-pyrrolizin-1-yl]-2,4,9,13- | | |
| | tetraoxatetradecan-1-oyl}piperazin- | | |
| | 1-yl)-4-oxo-1,4-dihydroquinoline- | | |
| | 3-carboxylic acid | | |
| 22 | 1-cyclopropyl-6-fluoro-7-(4- | 31.5 ± 2.6 | 66.7 ± 3.5 |
| | {[({[1-(4-{[2-(2-fluorobiphenyl- | Flurbiprofen | Gatifloxacin |
| | 4-yl)propanoyl]oxy}butanoyl) | | |
| | pyrrolidin-2-yl]carbonyl} | | |
| | oxy)methoxy]carbonyl}- | | |
| | 3-methylpiperazin-1-yl)- | | |
| | 8-methoxy-4-oxo-1,4- | | |
| | dihydroquinoline-3-carboxylic | | |
| | acid | | |

The data demonstrate that linkage of an antibioitic (e.g. gatifloxacin, moxifloxacin, and chloramphenicol, and tobramycin) and a non-steroidal anti-inflammatory (e.g. indomethacin, flurbiprofen, suprofen, ketorolac, and diclofenac) as a single hybrid compound is hydrolyzed enzymatically in rabbit cornea homogenates and human recombinant carboxylesterases to their respective individual antibiotic and non-steroidal anti-inflammatory drugs. These data suggest that these hybrid compounds will be cleaved in humans to the active metabolites to produce their respective pharmacologic effects.

### Example 2

### In Vitro Corneal Permeability and Metabolic Stability in Human Corneal Epithelial Cells

Clonetics® human corneal epithelial cells (HCEC) were purchased from Lonza Walkersville, Inc. (Walkersville, Md.) pre-seeded on Costar Transwell™ filters in a 24-well plate. Upon receipt HCEC cells were cultured overnight in a 37° C. incubator (95% O₂, 5% CO₂) in media provided by the vendor. Permeability studies were performed within 24 hours of receipt. Dosing solutions 100 μM test article (i.e. ester linked hybrids) were prepared in Lonza's proprietary media by diluting a 50 mM stock solution of the test article in dimethyl sulfoxide. The final percentage of solvent in the incubation was less than 1.0% to prevent inhibition of enzymatic activity or effects on the cell membrane. Transepithelial electrical resistance (TEER) was measured for all wells using a voltohmmeter with STX-2 electrodes (World Precision Instruments Inc., Sarasota, Fla.) after adding 100 μL pre-warmed (37° C.) media to the apical compartment. All permeability experiments were performed in triplicate by adding 100 μL of the 100 μM dosing solution to the apical compartment of each well (final incubation concentration of 50 μM). After a two hour incubation, aliquots of medium from the basolateral compartment of each well were removed to assess permeability. Aliquots of the dosing solution from the apical compartment of each well were collected at the end of incubation to assess mass balance. A final TEER value was measured and recorded for all wells.

To evaluate human corneal epithelial cell integrity incubations were conducted using 2 μCi/mL ³H-mannitol for the same 2 hour incubation period with aliquots taken from the basolateral compartment. ³H-Mannitol samples were analyzed using liquid scintillation counting. All ester linked hybrids samples were analyzed by liquid chromatography with mass spectrometry (LC-MS/MS) detection to determine the parent (i.e., ester linked hybrids) and metabolite (i.e. steroid and antibiotic) concentrations resulting from the metabolism of ester linked hybrids. Internal standards were used to compensate for variability from sample processing, chromatographic elution, mass spectrometer response and ion suppression by matrix components.

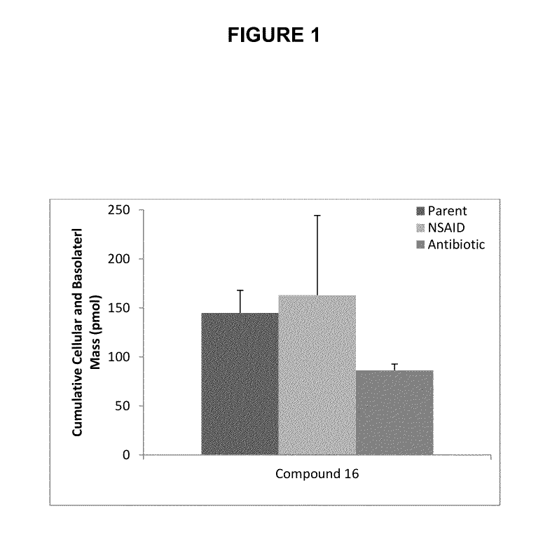

FIG. 1 shows the cellular uptake of ester linked hybrid, Compound 16 and the hydrolyzed metabolites [non-steroidal anti-inflammatory (NSAID) and antibiotic] after a two hour incubation with Human Corneal Epithelial Cells. The data demonstrate that linkage of an antibioitic (e.g. gatifloxacin) and a non-steroidal anti-inflammatory (e.g. flurbiprofen) as a single hybrid compound penetrates into human corneal epithelial cells and is enzymatically hydrolyzed to the individual antibiotic and NSAID. Therefore, providing evidence that when dosed in humans, the hybrid compound swill penetrate human ocular tissues and will be cleaved to the active metabolites to produce their respective pharmacologic effects.

What is claimed is:

1. A hybrid compound comprising an antibiotic moiety and a nonsteroidal anti-inflammatory drug moiety, or a pharmaceutical salt thereof, which are connected via two separate covalent bonds to a linker such that said covalent bonds degrade in vivo to yield the antibiotic and the nonsteroidal anti-inflammatory drug.

2. The hybrid compound according to claim 1 wherein, the covalent bonds can be ester bonds or amide bonds.

3. The hybrid compound according to claim 1 wherein, the nonsteroidal anti-inflammatory drug moiety is selected from the group consisting of indomethacin, diclofenac, flurbiprofen, ketorolac and suprofen.

4. The hybrid compound according to claim 1 wherein the antibiotic drug moiety is selected from the group consisting of: gatifloxacin, moxifloxacin, chloramphenicol, tobramycin and amikacin.

5. The hybrid compound according to claim 1 wherein said linker comprises an ester, a carboxylate, a carbonyl, a carbonate, an amido, a carbamate, a ketone, an amino, an oxo, an ethylene glycol, a polyethylene glycol moiety or an ethylene moiety.

6. The hybrid compound according to claim 1, comprising a gatifloxacin moiety and a nonsteroidal anti-inflammatory drug moiety selected from the group consisting of: indomethacin, diclofenac, flurbiprofen and suprofen.

7. The hybrid compound according to claim 1, comprising a moxifloxacin moiety and a nonsteroidal anti-inflammatory drug moiety selected from the group consisting of: indomethacin, diclofenac, and suprofen.

8. The hybrid compound according to claim 1, comprising a chloramphenicol moiety and a nonsteroidal anti-inflammatory drug moiety selected from the group consisting of: indomethacin and flurbiprofen.

9. The hybrid compound according to claim 1, comprising a tobramycin moiety and a nonsteroidal anti-inflammatory drug moiety selected from the group consisting of: indomethacin, flurbiprofen, and ketorolac.

10. The hybrid compound according to claim 1, comprising a amikacin moiety and flurbiprofen.

11. A pharmaceutical composition comprising a therapeutically effective amount of a hybrid compound, comprising an antibiotic moiety and a nonsteroidal anti-inflammatory drug moiety, which are connected via two separate covalent bonds to a linker such that said covalent bonds degrade in vivo to yield the antibiotic and the nonsteroidal anti-inflammatory drugs, and wherein said pharmaceutical composition is formulated for topical ophthalmic administration.

12. A method comprising administrating to an eye of a mammal a pharmaceutical composition comprising a therapeutically effective amount of a hybrid compound comprising an antibiotic moiety and a nonsteroidal anti-inflammatory drug moiety, which are connected via two separate covalent bonds to a linker such that said covalent bonds degrade in vivo to yield the antibiotic and the nonsteroidal anti-inflammatory drugs, and wherein said method is effective in the treatment of an inflammatory condition or bacterial infection affecting said eye.

13. The method according to claim 12 wherein said hybrid compound has topical antibiotic and nonsteroidal anti-inflammatory activity upon a surface of an eye, and wherein the hybrid compound degrades on said surface into said active antibiotic and said nonsteroidal anti-inflammatory drug, which are capable of penetrating beyond tissue of said surface

14. The hybrid compound according to claim 1 comprising a linker having two bonds, wherein said bonds are asymmetrically degraded in vivo to release the antibiotic and nonsteroidal anti-inflammatory drugs.

15. The hybrid compound according to claim 1, selected from:
7-[4-({1-[(4-chlorophenyl)carbonyl]-5-methoxy-2-methyl-1H-indol-3-yl}acetyl)-3-methylpiperazin-1-yl]-1-cyclopropyl-6-fluoro-8-methoxy-4-oxo-1,4-dihydroquinoline-3-carboxylic acid;
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(3-methylpiperazin-1-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(3-methylpiperazin-1-yl)-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl rel-1-cyclopropyl-7-[(4aR,7aR)-4-a,7a-dimethyloctahydro-6H-pyrrolo[3,4-b]pyridin-6-yl]-6-fluoro-8-methoxy-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl rel-1-cyclopropyl-7-[(4aR,7aR)-4a,7a-dimethyloctahydro-6H-pyrrolo[3,4-b]pyridin-6-yl]-6-fluoro-8-methoxy-4-oxo-1,4-dihydroquinoline-3-carboxylate;
2-({[1-cyclopropyl-6-fluoro-8-methoxy-7-(3-methyl piperazin-1-yl)-4-oxo-1,4-dihydroquinolin-3-yl]carbonyl}amino)ethyl 5-(phenylcarbonyl)-2,3-dihydro-1H-pyrrolizine-1-carboxylate;
2-[({1-cyclopropyl-7-[(4aR,7aR)-4a,7a-dimethyloctahydro-6H-pyrrolo[3,4-b]pyridin-6-yl]-6-fluoro-8-methoxy-4-oxo-1,4-dihydroquinolin-3-yl}carbonyl)amino]ethyl rel-5-(phenylcarbonyl)-2,3-dihydro-1H-pyrrolizine-1-carboxylate;
3-[({1-[(4-chlorophenyl)carbonyl]-5-methoxy-2-methyl-1H-indol-3-yl}acetyl)oxy]propyl 1-cyclopropyl-6-fluoro-8-methoxy-7-(3-methylpiperazin-1-yl)-4-oxo-1,4-dihydroquinoline-3-carboxylate;
2-[2-(2-{[2-(2-fluorobiphenyl-4-yl)propanoyl]oxy}ethoxy)ethoxy]ethyl 1-cyclopropyl-6-fluoro-8-methoxy-7-(3-methylpiperazin-1-yl)-4-oxo-1,4-dihydroquinoline-3-carboxylate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-4-{[2-(2-fluorobiphenyl-4-yl)propanoyl]oxy}butanoate;
2-[({2-[(dichloroacetyl)amino]-3-hydroxy-3-(4-nitrophenyl)propoxy}carbonyl)amino]ethyl {1-[(4-chlorophenyl)carbonyl]-5-methoxy-2-methyl-1H-indol-3-yl}acetate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-4-[({1-[(4-chlorophenyl)carbonyl]-5-methoxy-2-methyl-1H-indol-3-yl}acetyl)oxy]butanoate;
3-{[2-(2-fluorobiphenyl-4-yl)propanoyl]oxy}propyl 1-cyclopropyl-6-fluoro-8-methoxy-7-(3-methylpiperazin-1-yl)-4-oxo-1,4-dihydroquinoline-3-carboxylate;
2-{[2-(2-fluorobiphenyl-4-yl)propanoyl]oxy}ethyl 1-cyclopropyl-6-fluoro-8-methoxy-7-(3-methylpiperazin-1-yl)-4-oxo-1,4-dihydroquinoline-3-carboxylate;
2-{[2-(2-fluorobiphenyl-4-yl)propanoyl]oxy}ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate;
3-[({2-[(2,6-dichlorophenyl)amino]phenyl}acetyl)oxy]propyl 1-cyclopropyl-6-fluoro-8-methoxy-7-(3-methylpiperazin-1-yl)-4-oxo-1,4-dihydroquinoline-3-carboxylate;
3-[({2-[(2,6-dichlorophenyl)amino]phenyl}acetyl)oxy]propyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate;
2-[(dichloroacetyl)amino]-3-hydroxy-3-(4-nitrophenyl)propyl 4-[({1-[(4-chlorophenyl)carbonyl]-5-methoxy-2-methyl-1H-indol-3-yl}acetyl)oxy]butanoate;
2-[(dichloroacetyl)amino]-3-hydroxy-1-(4-nitrophenyl)propyl 4-{[2-(2-fluorobiphenyl-4-yl)propanoyl]oxy}butanoate;
2-[(dichloroacetyl)amino]-3-hydroxy-3-(4-nitrophenyl)propyl {1-[(4-chlorophenyl)carbonyl]-5-methoxy-2-methyl-1H-indol-3-yl}acetate;
1-cyclopropyl-6-fluoro-8-methoxy-7-(3-methyl-4-{[({[5-(phenylcarbonyl)-2,3-di hydro-1H-pyrrolizin-1-yl]carbonyl}oxy)methoxy]carbonyl}piperazin-1-yl)-4-oxo-1,4-dihydroquinoline-3-carboxylic acid;
7-[4-({[({1-[(4-chlorophenyl)carbonyl]-5-methoxy-2-methyl-1H-indol-3-yl}acetyl)oxy]methoxy}carbonyl)-3-methylpiperazin-1-yl]-1-cyclopropyl-6-fluoro-8-methoxy-4-oxo-1,4-dihydroquinoline-3-carboxylic acid;
rel-1-cyclopropyl-6-fluoro-8-methoxy-4-oxo-7-[(4aR,7aR)-1-{[({[5-(phenylcarbonyl)-2,3-dihydro-1H-pyrrolizin-1-yl]carbonyl}oxy)methoxy]carbonyl}octahydro-6H-pyrrolo[3,4-b]pyridin-6-yl]-1,4-dihydroquinoline-3-carboxylic acid;
3-{[2-(2-fluorobiphenyl-4-yl)propanoyl]oxy}propyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
[({1-[(4-chlorophenyl)carbonyl]-5-methoxy-2-methyl-1H-indol-3-yl}acetyl)oxy]methyl 1-cyclopropyl-6-fluoro-8-methoxy-7-(3-methylpiperazin-1-yl)-4-oxo-1,4-dihydroquinoline-3-carboxylate;
({[5-(phenylcarbonyl)-2,3-dihydro-1H-pyrrolizin-1-yl]carbonyl}oxy)methyl 1-cyclopropyl-6-fluoro-8-methoxy-7-(3-methylpiperazin-1-yl)-4-oxo-1,4-dihydroquinoline-3-carboxylate
2-[({1-[(4-chlorophenyl)carbonyl]-5-methoxy-2-methyl-1H-indol-3-yl}acetyl)oxy]ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
2-{[2-(2-fluorobiphenyl-4-yl)propanoyl]oxy}ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
[({1-[(4-chlorophenyl)carbonyl]-5-methoxy-2-methyl-1H-indol-3-yl}acetyl)oxy]methyl rel-1-cyclopropyl-6-fluoro-8-methoxy-7-[(4aR,7aR)-octahydro-6H-pyrrolo[3,4-b]pyridin-6-yl]-4-oxo-1,4-dihydroquinoline-3-carboxylate;
[({3-[({1-[(4-chlorophenyl)carbonyl]-5-methoxy-2-methyl-1H-indol-3-yl}acetyl)oxy]propoxy}carbonyl)oxy]methyl 1-cyclopropyl-6-fluoro-8-methoxy-7-(3-methylpiperazin-1-yl)-4-oxo-1,4-dihydroquinoline-3-carboxylate;
2-[2-(2-{[2-(2-fluorobiphenyl-4-yl)propanoyl]oxy}ethoxy)ethoxy]ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinoline-3-carboxylate;
3-[({1-[(4-chlorophenyl)carbonyl]-5-methoxy-2-methyl-1H-indol-3-yl}acetyl)oxy]propyl({[1-cyclopropyl-6-fluoro-8-methoxy-7-(3-methylpiperazin-1-yl)-4-oxo-1,4-dihydroquinolin-3-yl]carbonyl}oxy)methyl butanedioate;
({[1-cyclopropyl-6-fluoro-8-methoxy-7-(3-methylpiperazin-1-yl)-4-oxo-1,4-dihydroquinolin-3-yl]carbonyl}oxy)methyl 3-{[2-(2-fluorobiphenyl-4-yl)propanoyl]oxy}propyl butanedioate;
{[(3-{[2-(2-fluorobiphenyl-4-yl)propanoyl]oxy}propoxy)carbonyl]oxy}methyl 1-cyclopropyl-6-fluoro-8-methoxy-7-(3-methylpiperazin-1-yl)-4-oxo-1,4-dihydroquinoline-3-carboxylate;
[5-({[2-(2-fluorobiphenyl-4-yl)propanoyl]oxy}methyl)-2-oxo-1,3-dioxol-4-yl]methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-(4-{[5-({[2-(2-fluorobiphenyl-4-yl)propanoyl]oxy}methyl)-2-oxo-1,3-dioxol-4-yl]methyl}-3-methylpiperazin-1-yl)-8-methoxy-4-oxo-1,4-dihydroquinoline-3-carboxylic acid;
{[2-(2-fluorobiphenyl-4-yl)propanoyl]oxy}methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-{4-[({[2-(2-fluorobiphenyl-4-yl)propanoyl]oxy}methoxy)carbonyl]-3-methylpiperazin-1-yl}-8-methoxy-4-oxo-1,4-dihydroquinoline-3-carboxylic acid;
7-(4-{[({[({1-[(4-chlorophenyl)carbonyl]-5-methoxy-2-methyl-1H-indol-3-yl}acetyl)oxy]acetyl}oxy)methoxy]carbonyl}-3-methyl piperazin-1-yl)-1-cyclopropyl-6-fluoro-8-methoxy-4-oxo-1,4-dihydroquinoline-3-carboxylic acid;
1-cyclopropyl-7-[4-({[({2-[(2,6-dichlorophenyl)amino]phenyl}acetyl)oxy]methoxy}carbonyl)-3-methylpiperazin-1-yl]-6-fluoro-8-methoxy-4-oxo-1,4-dihydroquinoline-3-carboxylic acid;
[({2-[(2,6-dichlorophenyl)amino]phenyl}acetyl)oxy]methyl 1-cyclopropyl-6-fluoro-8-methoxy-7-(3-methylpiperazin-1-yl)-4-oxo-1,4-dihydroquinoline-3-carboxylate;
[(4-{[2-(2-fluorobiphenyl-4-yl)propanoyl]oxy}butanoyl)oxy]methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-[4-({[(4-{[2-(2-fluorobiphenyl-4-yl)propanoyl]oxy}butanoyl)oxy]methoxy}carbonyl)-3-methylpiperazin-1-yl]-8-methoxy-4-oxo-1,4-dihydroquinoline-3-carboxylic acid;
2-{2-[(dichloroacetyl)amino]-3-hydroxy-3-(4-nitrophenyl)propoxy}-2-oxoethyl {1-[(4-chlorophenyl)carbonyl]-5-methoxy-2-methyl-1H-indol-3-yl}acetate;
1-cyclopropyl-6-fluoro-8-methoxy-7-(3-methyl-4-{[({2-[4-(thiophen-2-ylcarbonyl)phenyl]propanoyl}oxy)methoxy]carbonyl}piperazin-1-yl)-4-oxo-1,4-dihydroquinoline-3-carboxylic acid;
({2-[4-(thiophen-2-ylcarbonyl)phenyl]propanoyl}oxy)methyl 1-cyclopropyl-6-fluoro-8-methoxy-7-(3-methylpiperazin-1-yl)-4-oxo-1,4-dihydroquinoline-3-carboxylate;
({[1-cyclopropyl-6-fluoro-8-methoxy-7-(3-methylpiperazin-1-yl)-4-oxo-1,4-dihydroquinolin-3-yl]carbonyl}oxy)methyl{[2-(2-fluorobiphenyl-4-yl)propanoyl]oxy}methyl butanedioate;
1-cyclopropyl-6-fluoro-7-{1-[({[2-(2-fluorobiphenyl-4-yl)propanoyl]oxy}methoxy)carbonyl]octahydro-6H-pyrrolo[3,4-b]pyridin-6-yl}-8-methoxy-4-oxo-1,4-dihydroquinoline-3-carboxylic acid;
7-[1-({[({1-[(4-chlorophenyl)carbonyl]-5-methoxy-2-methyl-1H-inden-3-yl}acetyl)oxy]methoxy}carbonyl)octahydro-6H-pyrrolo[3,4-b]pyridin-6-yl]-1-cyclopropyl-6-fluoro-8-methoxy-4-oxo-1,4-dihydroquinoline-3-carboxylic acid;
1-cyclopropyl-6-fluoro-7-[1-({[(4-{[2-(2-fluorobiphenyl-4-yl)propanoyl]oxy}butanoyl)oxy]methoxy}carbonyl)octahydro-6H-pyrrolo[3,4-b]pyridin-6-yl]-8-methoxy-4-oxo-1,4-dihydroquinoline-3-carboxylic acid;
7-(1-{[({4-[({1-[(4-chlorophenyl)carbonyl]-5-methoxy-2-methyl-1H-inden-3-yl}acetyl)oxy]butanoyl}oxy)methoxy]carbonyl}octahydro-6H-pyrrolo[3,4-b]pyridin-6-yl)-1-cyclopropyl-6-fluoro-8-methoxy-4-oxo-1,4-dihydroquinoline-3-carboxylic acid;
7-(4-{[({4-[({1-[(4-chlorophenyl)carbonyl]-5-methoxy-2-methyl-1H-inden-3-yl}acetyl)oxy]butanoyl}oxy)methoxy]carbonyl}-3-methyl piperazin-1-yl)-1-cyclopropyl-6-fluoro-8-methoxy-4-oxo-1,4-dihydroquinoline-3-carboxylic acid;
1-cyclopropyl-6-fluoro-8-methoxy-4-oxo-7-{1-[({[4-({[5-(phenylcarbonyl)-2,3-dihydro-1H-pyrrolizin-1-yl]carbonyl}oxy)butanoyl]oxy}methoxy)carbonyl]octahydro-6H-pyrrolo[3,4-b]pyridin-6-yl}-1,4-dihydroquinoline-3-carboxylic acid;
1-cyclopropyl-6-fluoro-8-methoxy-7-{3-methyl-4-[({[4-({[5-(phenylcarbonyl)-2,3-dihydro-1H-pyrrol izin-1-yl]carbonyl}oxy)butanoyl]oxy}methoxy)carbonyl]piperazin-1-yl}-4-oxo-1,4-dihydroquinoline-3-carboxylic acid;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-2-(2-fluorobiphenyl-4-yl)propanoate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-{1-[(4-chlorophenyl)carbonyl]-5-methoxy-2-methyl-1H-indol-3-yl}acetate;
1-cyclopropyl-6-fluoro-7-{4-[15-(2-fluorobiphenyl-4-yl)-5,8,14-trioxo-2,4,9,13-tetraoxahexadecan-1-oyl]-3-methylpiperazin-1-yl}-8-methoxy-4-oxo-1,4-dihydroquinoline-3-carboxylic acid;
1-cyclopropyl-6-fluoro-7-{1-[15-(2-fluorobiphenyl-4-yl)-5,8,14-trioxo-2,4,9,13-tetraoxahexadecan-1-oyl]octahydro-6H-pyrrolo[3,4-b]pyridin-6-yl}-8-methoxy-4-oxo-1,4-dihydroquinoline-3-carboxylic acid;
1-cyclopropyl-6-fluoro-8-methoxy-7-(3-methyl-4-{5,8,14-trioxo-15-[4-(thiophen-2-ylcarbonyl)phenyl]-2,4,9,13-tetraoxahexadecan-1-oyl}piperazin-1-yl)-4-oxo-1,4-dihydroquinoline-3-carboxylic acid;
1-cyclopropyl-6-fluoro-8-methoxy-4-oxo-7-(1-{5,8,14-trioxo-15-[4-(thiophen-2-ylcarbonyl)phenyl]-2,4,9,13-tetraoxahexadecan-1-oyl}octahydro-6H-pyrrolo[3,4-b]pyridin-6-yl)-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 3-({2-[4-(thiophen-2-ylcarbonyl)phenyl]propanoyl}oxy)propyl butanedioate;
[(2R,3S,4S,5R,6S)-4-amino-6-{[(1S,2S,3R,4S,6R)-4-amino-6-{[(2S)-4-amino-2-hydroxybutanoyl]amino}-3-{[(2R,3R,4S,5S,6R)-6-(aminomethyl)-3,4,5-trihydroxytetrahydro-2H-pyran-2-yl]oxy}-2-hydroxycyclohexyl]oxy}-3,5-dihydroxytetrahydro-2H-pyran-2-yl]methyl rel-2-(2-fluorobiphenyl-4-yl)propanoate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl rel-5-(phenylcarbonyl)-2,3-dihydro-1H-pyrrolizine-1-carboxylate;
7-[4-({[(2-amino-3-{[2-(2-fluorobiphenyl-4-yl)propanoyl]oxy}propanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
7-{4-[7-(tert-butoxycarbonyl)-15-(2-fluorobiphenyl-4-yl)-5,9,14-trioxo-2,4,13-trioxa-8-azahexadecan-1-oyl]-3-methylpiperazin-1-yl}-1-cyclopropyl-6-fluoro-8-methoxy-4-oxo-1,4-dihydroquinoline-3-carboxylic acid;
7-{4-[7-carboxy-15-(2-fluorobiphenyl-4-yl)-5,9,14-trioxo-2,4,13-trioxa-8-azahexadecan-1-oyl]-3-methylpiperazin-1-yl}-1-cyclopropyl-6-fluoro-8-methoxy-4-oxo-1,4-dihydroquinoline-3-carboxylic acid;
7-{4-[7-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
1-cyclopropyl-6-fluoro-7-{4-[15-(2-fluorobiphenyl-4-yl)-5,8,14-trioxo-2,4,13-trioxa-9-azahexadecan-1-oyl]-3-methylpiperazin-1-yl}-8-methoxy-4-oxo-1,4-dihydroquinoline-3-carboxylic acid;
7-{4-[6-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
7-[4-(2-amino-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-[4-(2-[(tert-butoxycarbonyl)amino]-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-{4-[6-(carboxymethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
7-{4-[6-(2-tert-butoxy-2-oxoethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
4-[({[1-cyclopropyl-6-fluoro-8-methoxy-7-(3-methylpiperazin-1-yl)-4-oxo-1,4-dihydroquinolin-3-yl]carbonyl}oxy)methyl]1-(3-{[2-(2-fluorobiphenyl-4-yl)propanoyl]oxy}propyl) 2-aminobutanedioate;
({[1-(4-{[2-(2-fluorobiphenyl-4-yl)propanoyl]oxy}butanoyl)pyrrolidin-2-yl]carbonyl}oxy)methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-(4-{[({[1-(4-{[2-(2-fluorobiphenyl-4-yl)propanoyl]oxy}butanoyl)pyrrolidin-2-yl]carbonyl}oxy)methoxy]carbonyl}-3-methylpiperazin-1-yl)-8-methoxy-4-oxo-1,4-dihydroquinoline-3-carboxylic acid;
1-cyclopropyl-6-fluoro-8-methoxy-7-(3-methyl-4-{5,8,14-trioxo-14-[5-(phenylcarbonyl)-2,3-dihydro-1H-pyrrolizin-1-yl]-2,4,9,13-tetraoxatetradecan-1-oyl}piperazin-1-yl)-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 3-({[5-(phenylcarbonyl)-2,3-dihydro-1H-pyrrolizin-1-yl]carbonyl}oxy)propyl butanedioate;
2-amino-3-{[2-(2-fluorobiphenyl-4-yl)propanoyl]oxy}propyl 1-cyclopropyl-6-fluoro-8-methoxy-7-(3-methylpiperazin-1-yl)-4-oxo-1,4-dihydroquinoline-3-carboxylate;
7-{4-[({[2-amino-3-(4-{[2-(2-fluorobiphenyl-4-yl)propanoyl]oxy}phenyl)propanoyl]oxy}methoxy)carbonyl]-3-methylpiperazin-1-yl}-1-cyclopropyl-6-fluoro-8-methoxy-4-oxo-1,4-dihydroquinoline-3-carboxylic acid;
1-cyclopropyl-6-fluoro-7-{4-[(3-{[2-(2-fluorobiphenyl-4-yl)propanoyl]oxy}propoxy)carbonyl]-3-methylpiperazin-1-yl}-8-methoxy-4-oxo-1,4-dihydroquinoline-3-carboxylic acid;
7-[4-({[(3-carboxy-3-{[2-(2-fluorobiphenyl-4-yl)propanoyl]amino}propanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
7-{4-[7-amino-16-(2-fluorobiphenyl-4-yl)-5,9,15-trioxo-2,4,10,14-tetraoxaheptadecan-1-oyl]-3-methylpiperazin-1-yl}-1-cyclopropyl-6-fluoro-8-methoxy-4-oxo-1,4-dihydroquinoline-3-carboxylic acid;
7-{4-[7-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
1-cyclopropyl-6-fluoro-7-{4-[15-(2-fluorobiphenyl-4-yl)-5,8,14-trioxo-2,4,13-trioxa-9-azahexadecan-1-oyl]-3-methylpiperazin-1-yl}-8-methoxy-4-oxo-1,4-dihydroquinoline-3-carboxylic acid;
7-{4-[6-amino-15-(2-fluorobiphenyl-4-yl)-5,8,14-trioxo-2,4,9,13-tetraoxahexadecan-1-oyl]-3-methylpiperazin-1-yl}-1-cyclopropyl-6-fluoro-8-methoxy-4-oxo-1,4-dihydroquinoline-3-carboxylic acid;
7-[4-(2-amino-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-[4-(2-[(tert-butoxycarbonyl)amino]-3-{[2-(2-fluorobiphenyl-4-yl)propanoyl]oxy}propanoyl)-3-methylpiperazin-1-yl]-1-cyclopropyl-6-fluoro-8-methoxy-4-oxo-1,4-dihydroquinoline-3-carboxylic acid;
7-{4-[6-(carboxymethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
7-{4-[6-(2-tert-butoxy-2-oxoethyl)-14-(2-fluorobiphenyl-4-yl)-5,8,13-trioxo-2,4,12-trioxa-7-azapentadecan-1-oyl]-3-methylpiperazin-1-yl}-1-cyclopropyl-6-fluoro-8-methoxy-4-oxo-1,4-dihydroquinoline-3-carboxylic acid;
4-[({[1-cyclopropyl-6-fluoro-8-methoxy-7-(3-methylpiperazin-1-yl)-4-oxo-1,4-dihydroquinolin-3-yl]carbonyl}oxy)methyl]1-(3-{[2-(2-fluorobiphenyl-4-yl)propanoyl]oxy}propyl) 2-aminobutanedioate;
({[1-(4-{[2-(2-fluorobiphenyl-4-yl)propanoyl]oxy}butanoyl)pyrrolidin-2-yl]carbonyl}oxy)methyl 1-cyclopropyl-6-fluoro-8-methoxy-7-(3-methylpiperazin-1-yl)-4-oxo-1,4-dihydroquinoline-3-carboxylate;
1-cyclopropyl-6-fluoro-7-(4-{[({[1-(4-{[2-(2-fluorobiphenyl-4-yl)propanoyl]oxy}butanoyl)pyrrolidin-2-yl]carbonyl}oxy)methoxy]carbonyl}-3-methylpiperazin-1-yl)-8-methoxy-4-oxo-1,4-dihydroquinoline-3-carboxylic acid;
1-cyclopropyl-6-fluoro-8-methoxy-7-(3-methyl-4-{5,8,14-trioxo-14-[5-(phenylcarbonyl)-2,3-dihydro-1H-pyrrolizin-1-yl]-2,4,9,13-tetraoxatetradecan-1-oyl}piperazin-1-yl)-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 3-({[5-(phenylcarbonyl)-2,3-dihydro-1H-pyrrolizin-1-yl]carbonyl}oxy)propyl butanedioate;
({[1-cyclopropyl-6-fluoro-8-methoxy-7-(3-methylpiperazin-1-yl)-4-oxo-1,4-dihydroquinolin-3-yl]carbonyl}oxy)methyl{[2-(2-fluorobiphenyl-4-yl)propanoyl]oxy}methyl butanedioate;
2-[({7-[4-(tert-butoxycarbonyl)-3-methylpiperazin-1-yl]-1-cyclopropyl-6-fluoro-8-methoxy-4-oxo-1,4-dihydroquinolin-3-yl}carbonyl)amino]ethyl 5-(phenylcarbonyl)-2,3-dihydro-1H-pyrrolizine-1-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 7-[1-(tert-butoxycarbonyl)octahydro-6H-pyrrolo[3,4-b]pyridin-6-yl]-1-cyclopropyl-6-fluoro-8-methoxy-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(octahydro-6H-pyrrolo[3,4-b]pyridin-6-yl)-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl-7-[1-(tert-butoxycarbonyl)octahydro-6H-pyrrolo[3,4-b]pyridin-6-yl]-1-cyclopropyl-6-fluoro-8-methoxy-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 1-cyclopropyl-6-fluoro-8-methoxy-7-(3-methylpiperazin-1-yl)-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl 7-[4-(tert-butoxycarbonyl)-3-methylpiperazin-1-yl]-1-cyclopropyl-6-fluoro-8-methoxy-4-oxo-1,4-dihydroquinoline-3-carboxylate;
2-{2-[2-({[5-(phenylcarbonyl)-2,3-dihydro-1H-pyrrolizin-1-yl]carbonyl}oxy)ethoxy]ethoxy}ethyl 1-cyclopropyl-6-fluoro-8-methoxy-7-(3-methylpiperazin-1-yl)-4-oxo-1,4-dihydroquinoline-3-carboxylate;
2-(2-{2-[({1-[(4-chlorophenyl)carbonyl]-5-methoxy-2-methyl-1H-indol-3-yl}acetyl)oxy]ethoxy}ethoxy)ethyl 7-[4-(tert-butoxycarbonyl)-3-methylpiperazin-1-yl]-1-cyclopropyl-6-fluoro-8-methoxy-4-oxo-1,4-dihydroquinoline-3-carboxylate; and
7-[4-({1-[(4-chlorophenyl)carbonyl]-5-methoxy-2-methyl-1H-indol-3-yl}acetyl)-3-methylpiperazin-1-yl]-1-cyclopropyl-6-fluoro-8-methoxy-4-oxo-1,4-dihydroquinoline-3-carboxylic acid.
